SEQUENCE LISTING (46 sequences)

SEQ ID NO: 1 - DNA, 972 bp, Novosphingobium aromaticivorans
  source 1..972: mol_type = genomic DNA
atgaccgaccggaaagcccttgtcggaatagcccgcaagtcgatcctcaagggctcgaagagcttcgctgcggccagccacctgttcgaccgtgaaacgcgcgagcgcgtctggctgctctacgcctggtgccgccggtgcgacgatatcgtcgatgcgcaggaccatggcggcgcgctgggaccgcaagacggcatgcaggagcggctccagcttgtccgcgagaagaccgccctcgcctttgccggacgcgagacgggcgagccggcgttcgatgcactgggcgtcgtggcgcgcgagacggggctgacgctggaaatggcgggcgccgtcatcgagggtttcgcgctcgacgcggccgactggcaaccgcattcggaagcggacctcatgcgctactgctggcacgtggccggggccgtgggtgtgatgatggcggtggtgatgggggtttcgcccgacgatgccgacacgctggaccgcgcctgcgatctgggactggcgttccagcttgccaatatcgcccgagacatcgaggaggatgacgccgccggccgctgctacatcccgcttgactggatggccgaggaagacatcccgccgggcgagcagatgaagccgcactaccggcccgcgctgacccggatcgtcgcgcgcatgtgccgtaccgcccacgtccaccagtgctctgcgcggatcggcgcggcgcggctgaaagggcgccagcgctgggcgattctcgccgcggcaggcatctatggcgagatcgcgcgcgaagtggaacggcgcggcgaccacgcgtgggaccaccgcaccgtcgtcagcaacgtgcgcaagctgggctttatcggcaaagccgcgtgggcggcggtccgccctgttccacgctcgtgcctggccgccgccagcgagcgcaagatgagccgacaggatctcatggaccttgcccgccatacgaggccgctggcctga

SEQ ID NO: 2 - protein, 323 aa, Novosphingobium aromaticivorans
MTDRKALVGIARKSILKGSKSFAAASHLFDRETRERVWLLYAWCRRCDDIVDAQDHGGALGPQDGMQERLQLVREKTALAFAGRETGEPAFDALGVVARETGLTLEMAGAVIEGFALDAADWQPHSEADLMRYCWHVAGAVGVMMAVVMGVSPDDADTLDRACDLGLAFQLANIARDIEEDDAAGRCYIPLDWMAEEDIPPGEQMKPHYRPALTRIVARMCRTAHVHQCSARIGAARLKGRQRWAILAAAGIYGEIAREVERRGDHAWDHRTVVSNVRKLGFIGKAAWAAVRPVPRSCLAAASERKMSRQDLMDLARHTRPLA

SEQ ID NO: 3 - DNA, 1479 bp, Novosphingobium aromaticivorans
  source 1..1479: mol_type = genomic DNA
atgaagcgtgcatgtgtcatcggcgccggatttggcggcctcgccctcgccatccgcctgcaatcggggggcgtccagacgaccctgatcgaggctcgcgacaagccgggcgggcgcgcctatttctgggagaaggacggcttcaccttcgacggcggcccgaccgtcatcaccgatccggcgtgcctggaagagctgtgggcgctcaagggccatcgcatggccgacgacgtggaactcatgccggtcatgccgttctaccgcctcaactgggcggacggcaccaacttcgactattccaacgacgacgcgagcctgcgcgctgagatcgccaagctggagccggcggacgttgccggatacgacgatttcctgcgctattccgccggcgtgttcgaggaaggctacgtcaagctcggcgccgtgccgttcctcgacttcgcgagcatgatcaaggccgcgcccgcgctggcccgctaccaggcctggcgctcggtctattcgatggtctccagcttcgtccgcaacgagaagctgcgcgaggccttcagtttccacactctgctggtcggcggaaacccgatgacgaccagcgcgatctatgcgctgatccacaagctggagaaagacggcggcgtgtggtgggccaagggcggcaccaaccgcctgatcgagggcatggtgacccatttccggcggatcggcggggaagtgcggatcggcgatccggtaacgcgcgtcgagacgctgggaacccgggtgaccggggtcgagacccgcagcggctggtcgggcaagttcgacgccgtagcgtcgaatgccgacgtcatgcattcgtaccgcgaccttctgtctgacaactcacatgcaaaacgccgcgcaaagtctctcggcaaaaagagattttcaccaagcctcttcgtcgtccacttcggcatcgaaggaacctggcccggtatcccgcatcacatgatcctgttcggcccgcgctacaaggggctgctcgacgacatctacacccatggcgtgctgccgcaggacttctcgatctacctgcaccatcccaccgtgacggacccctcggtcgcgcccgaaggtatgagcacgttctacgcgctggtgccggtggcgaacatgggcaagttgccggtcaactgggacgaggtcggtcccatcctcgagaagcgcatactggatgaagtgggccgcaggctcatccccgatatccacacgcgtatcgtaacgaagttccattacgctccgagcgacttctcgaccgatctttccgcgcacctcggaagtgcattcagccttgaaccgctgctcacgcaaagtgcatggttccgggcacataatcgcgacgacgcgatttccaatttctaccttgtcggcgcggggacgcaccccggtgccggcattccgggcgtggtcggctctgccaaggccacggcaaggctgatgctggaggatctttcgtga

SEQ ID NO: 4 - protein, 492 aa, Novosphingobium aromaticivorans
MKRACVIGAGFGGLALAIRLQSGGVQTTLIEARDKPGGRAYFWEKDGFTFDGGPTVITDPACLEELWALKGHRMADDVELMPVMPFYRLNWADGTNFDYSNDDASLRAEIAKLEPADVAGYDDFLRYSAGVFEEGYVKLGAVPFLDFASMIKAAPALARYQAWRSVYSMVSSFVRNEKLREAFSFHTLLVGGNPMTTSAIYALIHKLEKDGGVWWAKGGTNRLIEGMVTHFRRIGGEVRIGDPVTRVETLGTRVTGVETRSGWSGKFDAVASNADVMHSYRDLLSDNSHAKRRAKSLGKKRFSPSLFVVHFGIEGTWPGIPHHMILFGPRYKGLLDDIYTHGVLPQDFSIYLHHPTVTDPSVAPEGMSTFYALVPVANMGKLPVNWDEVGPILEKRILDEVGRRLIPDIHTRIVTKFHYAPSDFSTDLSAHLGSAFSLEPLLTQSAWFRAHNRDDAISNFYLVGAGTHPGAGIPGVVGSAKATARLMLEDLS

SEQ ID NO: 5 - DNA, 1197 bp, Novosphingobium aromaticivorans
  source 1..1197: mol_type = genomic DNA
atgaccgcgagacgctgcgacattgcgatccttggcggagggcttgcgggcggcctcgtggctctcgccctgcgccgtgcacgtcccgacctctcgctgctgctggtcgagcagggcgagcggctcggcggcaatcacgtgtggtcgttcttcggcagcgacgtgggcaacgaaggccgcgccttgctcgacggcatgtttgtcgccgcatggcccgaatataccgtccacttccccgctttccgccgacggctgaagacgagctattattcgatcacatcgagacgctttgacgcggtgcttcgggccgcgctcgggcaggactcattgatcaccggggtccgcgcgctcgcgtgcagcgccaccaaggtgacgctttcggaaggcacccgcatcgaggcgggcgccgttatcgacgcgcggggcttgcgcaacctccagcacctcaccggcggatggcagaagttcgtcggccgccggctccggctcgcgcagccgcacagtctcgacgcgccgatcgtcaaggatgcttccgtcgaacagatcgacggctaccgcttcgtctattgcctgccctttgccgccgacgagatcttcgtcgaggacacctattactccgacagccccgcgctcgacgtggcggtgctgggcgagaggatcgacgcctatgtcgcggcgcgcgggtggcaggtggccgaggtccttggcgaggagcatggcgtgcttccggtggtagcgggcggaaacttcgacgccttctggcggtcgagcggaggcgcggtcgcccgcgccggtgttcgcgccggcctgttccaggcggtcaccagctattcgctgcccgatgccgtgcgctttgccctggccctcgcccggcaaccggacctcggcggcgaggctctggccgcgttcagcgaagcctacgcgcgccggcactggaagcgctcggcgttccaccgcgcactttccgccatgctgttcgcggcggcccggcccgagcatcgctaccgcgtgctcgaacgcttctaccgcctggaccgccgccttatcgagcgtttctatgccggtcggacgacccttctcgacaaggcgcgcattctttcgggcaagccgccggtaccggtaaccaaggccatcggcgtgctcaccggccttggcgcgcgccccaacccactttcatttgcaggaacccgcgcatga

SEQ ID NO: 6 - protein, 398 aa, Novosphingobium aromaticivorans
MTARRCDIAILGGGLAGGLVALALRRARPDLSLLLVEQGERLGGNHVWSFFGSDVGNEGRALLDGMFVAAWPEYTVHFPAFRRRLKTSYYSITSRRFDAVLRAALGQDSLITGVRALACSATKVTLSEGTRIEAGAVIDARGLRNLQHLTGGWQKFVGRRLRLAQPHSLDAPIVKDASVEQIDGYRFVYCLPFAADEIFVEDTYYSDSPALDVAVLGERIDAYVAARGWQVAEVLGEEHGVLPVVAGGNFDAFWRSSGGAVARAGVRAGLFQAVTSYSLPDAVRFALALARQPDLGGEALAAFSEAYARRHWKRSAFHRALSAMLFAAARPEHRYRVLERFYRLDRRLIERFYAGRTTLLDKARILSGKPPVPVTKAIGVLTGLGARPNPLSFAGTRA

SEQ ID NO: 7 - DNA, 531 bp, Novosphingobium aromaticivorans
  source 1..531: mol_type = genomic DNA
atgggcggggccatgcagacgcttgccgcgatcctcattgtcctgggcacggtcctcgcgatggaattcgtcgcgtggtcgagccacaagtacatcatgcacggcttcggctggggctggcaccgcgaccatcatgagccgcacgaagggtttctcgagaagaacgatctctacgccattgtcggcgcggcgctttcgatcctgatgttcgcgctgggcagcccgatgatcatgggcgcggatgcctggtggccgggcacgtggatcggcctgggcgtcctgttctatggcgtgatctatacgctggtgcatgacgggctggtccaccagcgctggttccgatgggtcccgaagcgcggctatgccaagcggctggtccaggcgcacaagcttcaccacgccacgatcggcaaggaaggcggcgtcagtttcggcttcgtcttcgcgcgcgaccctgcggtgctgaagcaggaactgcgggcccagcgcgaggcaggcatcgccgttctgcgcgaggcagtggacggctag

SEQ ID NO: 8 - protein, 176 aa, Novosphingobium aromaticivorans
MGGAMQTLAAILIVLGTVLAMEFVAWSSHKYIMHGFGWGWHRDHHEPHEGFLEKNDLYAIVGAALSILMFALGSPMIMGADAWWPGTWIGLGVLFYGVIYTLVHDGLVHQRWFRWVPKRGYAKRLVQAHKLHHATIGKEGGVSFGFVFARDPAVLKQELRAQREAGIAVLREAVDG

SEQ ID NO: 9 - DNA, 711 bp, Novosphingobium aromaticivorans
  source 1..711: mol_type = genomic DNA
atgaacggcgtgctggtctcggcctgggccatgacggtgatcgtcgccgtccgctatcttgcgaccagcggtcttttcgcctggctgacggccagggtgaggcccggccactatgccgggctcgaccggcagatccgctcggagatcgggtggagccttgccaccgcggcgatctatggcgtgccggcgggcgtggtcgcctggggctggcaggagcgggggtggacgcggatctacaccgatccgcatgccttgccgctctggtggatgccggtctcgctcctgctctacctcttcatccacgatacctggttctactggacccaccgctggatgcaccggccggccgtgttccgccgcatccatgccatccaccacgccagtcgcccgccgacggcgtgggccgcaatgaacttccacccggtcgaggccgccgtggtctcgctgctgatccccgcgctggtcttcgcgatcccgatccacgtcgcggttctggggctggtgctgctggtgatgacggtgatgggcgtgaccaaccacatgggctgggaaatgtttccacgcgcgctcgttcattcccgggcagggaactggctgataaccgcaagccaccaccaccgccatcacgagcaataccgctgcaactatggcctctacttccggttctgggaccggttgtgcggcaccgacaagggattgtccgccccatga

SEQ ID NO: 10 - protein, 236 aa, Novosphingobium aromaticivorans
MNGVLVSAWAMTVIVAVRYLATSGLFAWLTARVRPGHYAGLDRQIRSEIGWSLATAAIYGVPAGVVAWGWQERGWTRIYTDPHALPLWWMPVSLLLYLFIHDTWFYWTHRWMHRPAVFRRIHAIHHASRPPTAWAAMNFHPVEAAVVSLLIPALVFAIPIHVAVLGLVLLVMTVMGVTNHMGWEMFPRALVHSRAGNWLITASHHHRHHEQYRCNYGLYFRFWDRLCGTDKGLSAP

SEQ ID NO: 11 - DNA, 717 bp, Sphingomonas taxi
  source 1..717: mol_type = genomic DNA
atgagccccgatcgggggaatacgcgccacagcctgctgctcgccgccgcgatcggtgcggcctggctcgccatccatattggcggcatcttcttctggcagtggcgtgccgccacggtgccggtcgcacttctgctgatcgtggtgcaggcgtggctgagcaccggcctcttcatcgtcgcgcacgactgcatgcacggatcgttcgcacccggacgccgggcgtggaacgtcgtcgtcggcaccctgtgcctcggcgcctatgccggcctgtcctatcgcgcgctctacccgatgcaccacgcgcatcatgccgcgcccggcaccgaacacgatcccgacttccatgccgccgcgcctcgccgcgcgcttccgtggttcgtccatttcttccgcgggtactacacccatggccagatcctgcggatcacgcttgcggcgatcgtctacatcctgctcggcgcgtcgcttctcaacatcgtgctgttctgggcggtgccggcgctgctcgcgcttgcgcaattgtttctgttcggcacctatctgccccaccgtcacggcgagacgccgttcgccgacacgcacaacgcgcgcagcaactcgctgtcgccgctcgcctcgctggcgacctgcttccacttcggtgcctatcaccacgaacatcacctcagcccgcagactccgtggtggcagctcccgcacatcaagcgcggctga

SEQ ID NO: 12 - protein, 238 aa, Sphingomonas taxi
MSPDRGNTRHSLLLAAAIGAAWLAIHIGGIFFWQWRAATVPVALLLIVVQAWLSTGLFIVAHDCMHGSFAPGRRAWNVVVGTLCLGAYAGLSYRALYPMHHAHHAAPGTEHDPDFHAAAPRRALPWFVHFFRGYYTHGQILRITLAAIVYILLGASLLNIVLFWAVPALLALAQLFLFGTYLPHRHGETPFADTHNARSNSLSPLASLATCFHFGAYHHEHHLSPQTPWWQLPHIKRG

SEQ ID NO: 13 - DNA, 777 bp, Sphingomonas astaxanthinifaciens
  source 1..777: mol_type = genomic DNA
atggcagaacgccgtcgcccggcctatatggcacccatgctcagtgatgcgcagcgccgtcgccaggcgatgatcggccttggccttgccgcagcgatcaccgcagccttcgtcgcgcttcatgtctggtcggtcttcttccttccgcttgaaggagcaggctggtggcttgcgcttccgatcgtcgcagtgcaaacctggcttagcgtcggtctgttcatcgtcgcgcatgatgcaatgcatggcagccttgcaccgggccgccctgcgaccaaccttttctggggacggcttacgcttctgctctacgcgggcttctggttggaccgcctttcgcccaagcatttcgaccaccaccgccatgtcgggaccgagcgcgatcccgatttctcggtcgatcatccgacccgcttctggccctggtattatgccttcatgcggcgctatttcgggcttcgcgaatatctggtgctgaacgcgctggtgctggcctacgtgctggtgctgaaggcgccgctcggcaatctgctcctgttctgggcgctgccctcgatcctgtcctcgatccagctcttctatttcggcacctaccttccgcaccggcacgaggacgcgcccttcgccgaccagcacaatgcccgcagcaacgactttccggtctggctgtcgctgctgacctgcttccacttcggctatcaccgcgagcatcacctcagccccggcaccccgtggtggcagctgcctcgacgacggcgagagcttgcacttcctgcatga

SEQ ID NO: 14 - protein, 258 aa, Sphingomonas astaxanthinifaciens
MAERRRPAYMAPMLSDAQRRRQAMIGLGLAAAITAAFVALHVWSVFFLPLEGAGWWLALPIVAVQTWLSVGLFIVAHDAMHGSLAPGRPATNLFWGRLTLLLYAGFWLDRLSPKHFDHHRHVGTERDPDFSVDHPTRFWPWYYAFMRRYFGLREYLVLNALVLAYVLVLKAPLGNLLLFWALPSILSSIQLFYFGTYLPHRHEDAPFADQHNARSNDFPVWLSLLTCFHFGYHREHHLSPGTPWWQLPRRRRELALPA

SEQ ID NO: 15 - DNA, 891 bp, Novosphingobium aromaticivorans
  source 1..891: mol_type = genomic DNA
atgaccgaccagaaccgcatcgtaagctggcacgccaatccgtcgaagccgcgctacaccccgccgccgggcgcggtcgacgcgcattgccacgtcttcggcccgatggcgcagttccccttctcggccaaggccaagtacctgcccgaggacgccggccccgacatgctcttcgccctgcgcgaccacctcggcttcgagcgcaacgtgatcgtccaggcaagctgccacggcaccgacaacgccgccacgcttgatgccatcgccaggtcgaacggaaaggcgcgcggcgtcgccgtcgtcgatcccgccatctcggagacggacctccaggctctccacgaaggcggcatccgcggcatccgcttcaacttcctcaagcgcctcgtcgacgatgcgcccaaggacaagttcctcgaagtcgcccggcgcctgcccaagggttggcacgtcgtgatctatttcgaggccgacatcctcgaggaactgcgtccgttcatggatgcgatccccgtgccgctggtcatcgaccacatgggccgccccgatgtccgccagggccccgacggcgccgacatgaaggccttccgcaacttcctgaacagccgcgacgacatctggttcaaggccacctgccccgaccggctcgacgcgatcaaggaaggcggcgcgggcgatccgtggaacgcctttgccgatgccgtcgcgccgctcgttgccgactaccaggaccgggtcctgtgggggaccgactggccgcaccccaacatggacaccgagatccccgacgacggccatctcgtcgacatgatcccccgcatcgccccgaccgaggaactgcaacgcaagctcctcgtcgacaatccgatgcggctctactgggccgactga

SEQ ID NO: 16 - protein, 296 aa, Novosphingobium aromaticivorans
MTDQNRIVSWHANPSKPRYTPPPGAVDAHCHVFGPMAQFPFSAKAKYLPEDAGPDMLFALRDHLGFERNVIVQASCHGTDNAATLDAIARSNGKARGVAVVDPAISETDLQALHEGGIRGIRFNFLKRLVDDAPKDKFLEVARRLPKGWHVVIYFEADILEELRPFMDAIPVPLVIDHMGRPDVRQGPDGADMKAFRNFLNSRDDIWFKATCPDRLDAIKEGGAGDPWNAFADAVAPLVADYQDRVLWGTDWPHPNMDTEIPDDGHLVDMIPRIAPTEELQRKLLVDNPMRLYWAD

SEQ ID NO: 17 - DNA, 738 bp, Novosphingobium aromaticivorans
  source 1..738: mol_type = genomic DNA
atggccggcttcgtcctgatccatggttcctggcatggtggctggtgcttcgatcccgtcgccgaaatcctgcgcgcgcgcggccacacggtcgtcgcgccgacgctgcccgggatgggaggcacggcggaggaaatggcggcggtcacgctggatggctggggcgaatttgctgcgcagcattgtcgcgacctgaaggcacggggcgtcggccccgtggtcctcgccggccactcgcgcggaggccttgtcgtctccaccgcggccgagcgcgacccttccgcgatggacgccatcgtctacatatgcgcgatgatgctgccctcgggcatgagtcgcgccgggttcaaggaactggaaggcccgaaccccgctttcgacgcgatcatctcgaaggttcacggcggcatcgccacggtcatcgacacgcagaatgctgcaccggtctttgcacaaatttcgccgcccgatctggtcgaggcggcaatggcaaggctcgtggccgaaccccatgctccgcgttcgcagcagatcaaggtcacgccggaacgctggggcagcctcccgcgcacttacgtcgaatgcacgctcgaccgcaccattccgatcgaaagccagcgccgcatgatcgcgatgtcgccgggtgcgaacgtggtgactctggaggcggaccacagtccctatctgtcgaaaccgcaggaactggccgaggcgctggaagcggccattcccgcctga

SEQ ID NO: 18 - protein, 245 aa, Novosphingobium aromaticivorans
MAGFVLIHGSWHGGWCFDPVAEILRARGHTVVAPTLPGMGGTAEEMAAVTLDGWGEFAAQHCRDLKARGVGPVVLAGHSRGGLVVSTAAERDPSAMDAIVYICAMMLPSGMSRAGFKELEGPNPAFDAIISKVHGGIATVIDTQNAAPVFAQISPPDLVEAAMARLVAEPHAPRSQQIKVTPERWGSLPRTYVECTLDRTIPIESQRRMIAMSPGANVVTLEADHSPYLSKPQELAEALEAAIPA

SEQ ID NO: 19 - DNA, 813 bp, Novosphingobium aromaticivorans
  source 1..813: mol_type = genomic DNA
atggcggcatgctcggcaccatgcgagtctgaaccgatggccttgaaatactaccacgccgaaccgctggccaactctctcaagtcgatggtcccgctcaaggaaaagggcctcgcctacgagagcatctacgtcgatctgcacaagttcgagcagcatcagccgtggttcaccgcgatcaatcccgaaggccaggtgccggtgctcgaccatgacggcacgatcatcacgcacacgacggtgatcaacgaatacctcgaggatgccttccccgatgcccagcccgccgatgcgcccctgcgtccgcgcgacccggtgggtgcggcgcgcatgcgctactggaacaagttcatcgacgagcacgtgatgaactacgtctcgatgcacggatggcaccgcatggtcggcgtgatcgcccgcaacatcgccagcggcgatttcgagaaactgctcgaaagcattccgctgcccgatcagcgcaagaagtgggctaccgcgcgatcgggcttttccgaagccgatctcgccaatgccaccgccaagatcgaatacgcgctcgacaaggtcgagaagcaactcggcgagacgaagtggctggcgggcgacacctacacgcttgccgacatcaacttctattcgcactgcggcgcgatggtcgaacgcatgttcccggaaatggaagtggcgaggcgcgcgccgcgcctgtgcgaatggcgtgatcgcgttgccgcgcggcctgccgtcgccgaagcgctgaaaagcgaagaccgcactgcgcccgggctgcgcgtctggtcgggagaagtgcgctga

SEQ ID NO: 20 - protein, 270 aa, Novosphingobium aromaticivorans
MAACSAPCESEPMALKYYHAEPLANSLKSMVPLKEKGLAYESIYVDLHKFEQHQPWFTAINPEGQVPVLDHDGTIITHTTVINEYLEDAFPDAQPADAPLRPRDPVGAARMRYWNKFIDEHVMNYVSMHGWHRMVGVIARNIASGDFEKLLESIPLPDQRKKWATARSGFSEADLANATAKIEYALDKVEKQLGETKWLAGDTYTLADINFYSHCGAMVERMFPEMEVARRAPRLCEWRDRVAARPAVAEALKSEDRTAPGLRVWSGEVR

SEQ ID NO: 21 - DNA, 1071 bp, Novosphingobium aromaticivorans
  source 1..1071: mol_type = genomic DNA
atgagcggcggcgaaccggttccgaagctgtcggcgaaacccgccgccacgtatcttcgcaacacctggtacgtggcgggctgggccagcgatcttgccggcgagccgcagcagcgcacgttcctggaagagccggtggcgctcttccgcgacggacacggtgaggcgaaggccatcggcgggcgctgcccgcaccggttcgcgccgctcggccatggcagcgtcgtcgacggggcgctgatgtgcccctaccacggcctgcgtttcgatggggatggacgctgcgtccacaacccgcatcccggcggacatcttcccgatgcgcggcagcgggtctatccgcttgtcgagcggcatgccttgctgtggatatggatgggcgatgcagcaaaggctgatccggcatcgatcccggacttttcgtggctttcggaccccagatgggaggccgtgcgcggggccacggtcgccgagggtcacttcgagctctacagcgacaacattctcgacctcagccacgccaacttcgtccacccggcgctggtcgccagcgcattcaccgaaggcgagcgcaagttctggcaggacggagacaatgtctttgccgaatacgtgcggctgaacgacgagctttccgtcggcatttcggcggtgatggggaccgaggggcggccgcaggatttctacggcatggtcaagtggcatgcgccggccgtactctacttcgatttccgcgcgggcgagccgggcacgccgcgcgagcaatgcacgctgctgccatcgctccatgccttcacgccggaaacccctgacacgacgcattacttctgggcgaccgcgcgcgactacaggctgggcgacgcggagttcaccgccggaatgcgcgccgcgctcgaattcgcgttcgagcaggaagacatgccgatcatccgcgacagccaccggctcatgcgcggcgaggacttctgggcgcttcgcccgctgatcctcggtggcgatggtggcggggtgcgggcccggagaatgctgcaacggctgatcgagcgcgagagacagcaggacgctgcctga

SEQ ID NO: 22 - protein, 356 aa, Novosphingobium aromaticivorans
MSGGEPVPKLSAKPAATYLRNTWYVAGWASDLAGEPQQRTFLEEPVALFRDGHGEAKAIGGRCPHRFAPLGHGSVVDGALMCPYHGLRFDGDGRCVHNPHPGGHLPDARQRVYPLVERHALLWIWMGDAAKADPASIPDFSWLSDPRWEAVRGATVAEGHFELYSDNILDLSHANFVHPALVASAFTEGERKFWQDGDNVFAEYVRLNDELSVGISAVMGTEGRPQDFYGMVKWHAPAVLYFDFRAGEPGTPREQCTLLPSLHAFTPETPDTTHYFWATARDYRLGDAEFTAGMRAALEFAFEQEDMPIIRDSHRLMRGEDFWALRPLILGGDGGGVRARRMLQRLIERERQQDAA

SEQ ID NO: 23 - DNA, 55 bp, synthetic construct
  source 1..55: mol_type = other DNA
cgattcattaatgcagctggcacgacagcaggactctcgatctacctgcaccatc

SEQ ID NO: 24 - DNA, 42 bp, synthetic construct
  source 1..42: mol_type = other DNA
cgataaagcccagcttgctcacaggtcgtcggccttcattgc

SEQ ID NO: 25 - DNA, 44 bp, synthetic construct
  source 1..44: mol_type = other DNA
gaaggccgacgacctgtgagcaagctgggctttatcggcaaagc

SEQ ID NO: 26 - DNA, 57 bp, synthetic construct
  source 1..57: mol_type = other DNA
gtttctgcggactggctttctagatgttccaccatgacgaggtggaccagaatgaac

SEQ ID NO: 27 - DNA, 59 bp, synthetic construct
  source 1..59: mol_type = other DNA
cgattcattaatgcagctggcacgacagcttgaaacggtagccgaaggtgtaaaggtcg

SEQ ID NO: 28 - DNA, 44 bp, synthetic construct
  source 1..44: mol_type = other DNA
gcaaatgaaagtgggttggcgatccgcttagggacatgcggttg

SEQ ID NO: 29 - DNA, 45 bp, synthetic construct
  source 1..45: mol_type = other DNA
catgtccctaagcggatcgccaacccactttcatttgcaggaacc

SEQ ID NO: 30 - DNA, 57 bp, synthetic construct
  source 1..57: mol_type = other DNA
gtttctgcggactggctttctagatgttcgatggtgcaggtagatcgagaagtcctg

SEQ ID NO: 31 - DNA, 58 bp, synthetic construct
  source 1..58: mol_type = other DNA
cgattcattaatgcagctggcacgacaggtcgaacagtacgtcaccttcatcaaccag

SEQ ID NO: 32 - DNA, 39 bp, synthetic construct
  source 1..39: mol_type = other DNA
cggtattgctcgtgatgccaacggctcctgcctgaacag

SEQ ID NO: 33 - DNA, 43 bp, synthetic construct
  source 1..43: mol_type = other DNA
gcaggagccgttggcatcacgagcaataccgctgcaactatgg

SEQ ID NO: 34 - DNA, 57 bp, synthetic construct
  source 1..57: mol_type = other DNA
gtttctgcggactggctttctagatgttcctcgtatcccacagcgatatcaggatgc

SEQ ID NO: 35 - DNA, 55 bp, synthetic construct
  source 1..55: mol_type = other DNA
cgattcattaatgcagctggcacgacagcacttccatcgtcttcgactgcttgag

SEQ ID NO: 36 - DNA, 48 bp, synthetic construct
  source 1..48: mol_type = other DNA
cctgcttcagcaccgcagcgacactttcttacaatttgcccgaaagtc

SEQ ID NO: 37 - DNA, 45 bp, synthetic construct
  source 1..45: mol_type = other DNA
ggcaaattgtaagaaagtgtcgctgcggtgctgaagcaggaactg

SEQ ID NO: 38 - DNA, 56 bp, synthetic construct
  source 1..56: mol_type = other DNA
gtttctgcggactggctttctagatgttcctactgccggattttccggcatggaag

SEQ ID NO: 39 - DNA, 23 bp, synthetic construct
  source 1..23: mol_type = other DNA
ctgtcgtgccagctgcattaatg

SEQ ID NO: 40 - DNA, 29 bp, synthetic construct
  source 1..29: mol_type = other DNA
gaacatctagaaagccagtccgcagaaac

SEQ ID NO: 41 - DNA, 22 bp, synthetic construct
  source 1..22: mol_type = other DNA
ccaacggctcctgcctgaacag

SEQ ID NO: 42 - DNA, 29 bp, synthetic construct
  source 1..29: mol_type = other DNA
catcacgagcaataccgctgcaactatgg

SEQ ID NO: 43 - DNA, 795 bp, synthetic construct
  source 1..795: mol_type = other DNA
gtcaaccgcgtcaacctgttcaggcaggagccgttgggcatgagccccgatcgggggaatacgcgccacagcctgctgctcgccgccgcgatcggtgcggcctggctcgccatccatattggcggcatcttcttctggcagtggcgtgccgccacggtgccggtcgcacttctgctgatcgtggtgcaggcgtggctgagcaccggcctcttcatcgtcgcgcacgactgcatgcacggatcgttcgcacccggacgccgggcgtggaacgtcgtcgtcggcaccctgtgcctcggcgcctatgccggcctgtcctatcgcgcgctctacccgatgcaccacgcgcatcatgccgcgcccggcaccgaacacgatcccgacttccatgccgccgcgcctcgccgcgcgcttccgtggttcgtccatttcttccgcgggtactacacccatggccagatcctgcggatcacgcttgcggcgatcgtctacatcctgctcggcgcgtcgcttctcaacatcgtgctgttctgggcggtgccggcgctgctcgcgcttgcgcaattgtttctgttcggcacctatctgccccaccgtcacggcgagacgccgttcgccgacacgcacaacgcgcgcagcaactcgctgtcgccgctcgcctcgctggcgacctgcttccacttcggtgcctatcaccacgaacatcacctcagcccgcagactccgtggtggcagctcccgcacatcaagcgcggctgacatcacgagcaataccgctgcaactatggcctctacttc

SEQ ID NO: 44 - DNA, 855 bp, synthetic construct
  source 1..855: mol_type = other DNA
gtcaaccgcgtcaacctgttcaggcaggagccgttgggcatggcagaacgccgtcgcccggcctatatggcacccatgctcagtgatgcgcagcgccgtcgccaggcgatgatcggccttggccttgccgcagcgatcaccgcagccttcgtcgcgcttcatgtctggtcggtcttcttccttccgcttgaaggagcaggctggtggcttgcgcttccgatcgtcgcagtgcaaacctggcttagcgtcggtctgttcatcgtcgcgcatgatgcaatgcatggcagccttgcaccgggccgccctgcgaccaaccttttctggggacggcttacgcttctgctctacgcgggcttctggttggaccgcctttcgcccaagcatttcgaccaccaccgccatgtcgggaccgagcgcgatcccgatttctcggtcgatcatccgacccgcttctggccctggtattatgccttcatgcggcgctatttcgggcttcgcgaatatctggtgctgaacgcgctggtgctggcctacgtgctggtgctgaaggcgccgctcggcaatctgctcctgttctgggcgctgccctcgatcctgtcctcgatccagctcttctatttcggcacctaccttccgcaccggcacgaggacgcgcccttcgccgaccagcacaatgcccgcagcaacgactttccggtctggctgtcgctgctgacctgcttccacttcggctatcaccgcgagcatcacctcagccccggcaccccgtggtggcagctgcctcgacgacggcgagagcttgcacttcctgcatgacatcacgagcaataccgctgcaactatggcctctacttc

SEQ ID NO: 45 - DNA, 22 bp, synthetic construct
  source 1..22: mol_type = other DNA
ccaacggctcctgcctgaacag

SEQ ID NO: 46 - DNA, 29 bp, synthetic construct
  source 1..29: mol_type = other DNA
catcacgagcaataccgctgcaactatgg

(19) United States
(12) Patent Application Publication
(10) Pub. No.: US 2026/0125731 A1
(43) Pub. Date: May 7, 2026

(54) MICROORGANISMS AND METHODS FOR PRODUCING CAROTENOIDS AND OTHER COMPOUNDS

(71) Applicant: Wisconsin Alumni Research Foundation, Madison, WI (US)

(72) Inventors: Timothy Donohue, Middleton, WI (US); Daniel Noguera, Madison, WI (US); Benjamin Hall, Waukesha, WI (US); Wayne Kontur, Grove City, OH (US); Derek Gille, Chicago, IL (US); Jeanette Neri, Bull Valley, IL (US)

(73) Assignee: Wisconsin Alumni Research Foundation, Madison, WI (US)

(21) Appl. No.: 19/371,081

(22) Filed: Oct. 28, 2025

Related U.S. Application Data
(60) Provisional application No. 63/712,785, filed on Oct. 28, 2024.

(51) Int. Cl. C12P 23/00; C12N 9/02; C12N 9/10; C12N 9/18; C12N 9/90

(52) U.S. Cl. CPC C12P 23/00; C12N 9/001; C12N 9/0071; C12N 9/0077; C12N 9/0083; C12N 9/1007; C12N 9/1085; C12N 9/18; C12N 9/90; C12Y 103/99031; C12Y 205/01032; C12Y 301/01057; C12Y 505/01019

(57) ABSTRACT

Recombinant microorganisms configured for enhanced production of carotenoids and other compounds and methods of using the recombinant microorganisms for the production of same. The recombinant microorganism can include a modification that decreases phytoene synthase (CrtB) activity, a modification that decreases lycopene-forming phytoene desaturase (CrtI) activity, a modification that decreases lycopene cyclase (CrtY) activity, a modification that decreases beta-carotene hydroxylase (CrtZ) activity, a modification that decreases 2,2′-beta hydroxylase (CrtG) activity, and/or a modification that increases beta-carotene ketolase (CrtW) activity. The recombinant microorganism can be from the genus Novosphingobium, such as Novosphingobium aromaticivorans.

### CROSS-REFERENCE TO RELATED APPLICATIONS

Priority is hereby claimed to U.S. Provisional Application 63/712,785, filed Oct. 28, 2024, which is incorporated herein by reference in its entirety.

### STATEMENT REGARDING FEDERALLY SPONSORED RESEARCH

This invention was made with government support under DE-SC0018409 awarded by the US Department of Energy. The government has certain rights in the invention.

### SEQUENCE LISTING

The instant application contains a Sequence Listing which has been submitted in XML format and is hereby incorporated by reference in its entirety. The XML copy, created on Aug. 22, 2025, is named USPTO-250822-09824639-P230405US02-SEQ_LIST.xml and is 56,670 bytes in size.

### FIELD OF THE INVENTION

The invention is directed to recombinant microorganisms configured for enhanced production of carotenoids and other compounds and methods of using the recombinant microorganisms for the production of same.

### BACKGROUND

Carotenoids are lipophilic isoprenoids that are produced by some plants, algae, bacteria, and fungi, and function as membrane-bound light-harvesting pigments and antioxidants (8, 9). Several carotenoids (including astaxanthin, β-carotene, lycopene, and zeaxanthin) are used industrially as animal feed, food coloring, nutritional supplements, cosmetics additives, and pharmaceuticals, with a 2017 global market size of ˜$1.5B (10, 11). Most industrial carotenoids are produced synthetically (9, 10, 12), though there are a few biological sources commercially being used, such as the flower Tagetes erecta for lutein and the alga Dunaliella salina for 3-carotene (10). Thus, there is growing interest in developing new biological sources of carotenoids (9, 10). New sources for the production of carotenoids such as astaxanthin, β-carotene, lycopene, and zeaxanthin are needed.

### SUMMARY OF THE INVENTION

One aspect of the invention is directed to recombinant microorganisms comprising one or more modifications with respect to a corresponding microorganism not comprising the one or more modifications.

In some versions, the one or more modifications comprise any one or more of: a modification that decreases phytoene synthase (CrtB) activity with respect to the corresponding microorganism; a modification that decreases lycopene-forming phytoene desaturase (CrtI) activity with respect to the corresponding microorganism; a modification that decreases lycopene cyclase (CrtY) activity with respect to the corresponding microorganism; a modification that decreases beta-carotene hydroxylase (CrtZ) activity with respect to the corresponding microorganism; a modification that decreases 2,2′-beta hydroxylase (CrtG) activity with respect to the corresponding microorganism; and a modification that increases beta-carotene ketolase (CrtW) activity with respect to the corresponding microorganism.

In some versions, the modification that decreases phytoene synthase (CrtB) activity comprises a modification to a gene encoding a phytoene synthase (CrtB) comprising a sequence having at least 80%, at least 85%, at least 90%, at least 95%, or at least 99% sequence identity to SEQ ID NO:2.

In some versions, the modification that decreases lycopene-forming phytoene (CrtI) desaturase activity comprises a modification to a gene encoding a lycopene-forming phytoene desaturase (CrtI) comprising a sequence having at least 80%, at least 85%, at least 90%, at least 95%, or at least 99% sequence identity to SEQ ID NO:4.

In some versions, the modification that decreases lycopene cyclase (CrtY) activity comprises a modification to a gene encoding a lycopene cyclase (CrtY) comprising a sequence having at least 80%, at least 85%, at least 90%, at least 95%, or at least 99% sequence identity to SEQ ID NO:6.

In some versions, the modification that decreases beta-carotene hydroxylase (CrtZ) activity comprises a modification to a gene encoding a beta-carotene hydroxylase (CrtZ) comprising a sequence having at least 80%, at least 85%, at least 90%, at least 95%, or at least 99% sequence identity to SEQ ID NO:8.

In some versions, the modification that decreases 2,2′-beta hydroxylase (CrtG) activity comprises a modification to a gene encoding a 2,2′-beta hydroxylase (CrtG) comprising a sequence having at least 80%, at least 85%, at least 90%, at least 95%, or at least 99% sequence identity to SEQ ID NO:10.

In some versions, the modification that increases beta-carotene ketolase (CrtW) activity comprises a recombinant gene encoding a beta-carotene ketolase (CrtW) comprising a sequence having at least 80%, at least 85%, at least 90%, at least 95%, or at least 99% sequence identity to SEQ ID NO:12 or SEQ ID NO:14.

In some versions, the recombinant microorganism comprises any one or more of: a modification that decreases phytoene synthase (CrtB) activity; a modification that decreases lycopene-forming phytoene desaturase (CrtI) activity; a modification that decreases lycopene cyclase (CrtY) activity; a modification that decreases beta-carotene hydroxylase (CrtZ) activity; and a modification that decreases 2,2′-beta hydroxylase (CrtG) activity. In some versions, the recombinant microorganism comprises any one or more of: a modification that decreases phytoene synthase (CrtB) activity; a modification that decreases lycopene-forming phytoene desaturase (CrtI) activity; and a modification that decreases lycopene cyclase (CrtY) activity. In some versions, the recombinant microorganism comprises a modification that decreases phytoene synthase (CrtB) activity. In some of the foregoing versions, the recombinant microorganism exhibits increased accumulation of coenzyme Q₁₀ (CoQ₁₀) with respect to the corresponding microorganism.

In some versions, the microorganism comprises a modification that decreases lycopene cyclase (CrtY) activity. In some of such versions, the recombinant microorganism exhibits increased accumulation of lycopene with respect to the corresponding microorganism.

In some versions, recombinant microorganism comprises a modification that decreases 2,2′-beta hydroxylase (CrtG) activity. In some such versions, the recombinant microorganism exhibits increased accumulation of zeaxanthin with respect to the corresponding microorganism.

In some versions, the recombinant microorganism comprises a modification that decreases 2,2′-beta hydroxylase (CrtG) activity and/or a modification that decreases beta-carotene hydroxylase (CrtZ) activity. In some such versions, the recombinant microorganism exhibits increased accumulation of beta-carotene with respect to the corresponding microorganism.

In some versions, the recombinant microorganism comprises a modification that decreases 2,2′-beta hydroxylase (CrtG) activity and/or a modification that increases beta-carotene ketolase (CrtW) activity. In some such versions, the recombinant microorganism exhibits increased accumulation of at least one of adonixanthin and astaxanthan with respect to the corresponding microorganism.

In some versions, the recombinant microorganism further comprises: a modification that decreases 2-pyrone-4,6-dicarboxylic acid (PDC) hydrolase (LigI) activity with respect to the corresponding microorganism; a modification that decreases 4-carboxy-2-hydroxy-6-methoxy-6-oxohexa-2,4-dienoate (CHMOD) methyl esterase (DesC) activity with respect to the corresponding microorganism; a modification that decreases 4-carboxy-2-hydroxy-6-methoxy-6-oxohexa-2,4-dienoate (CHMOD) cis-trans isomerase (DesD) activity with respect to the corresponding microorganism; and/or a modification that decreases vanillate/3-O-methylgallate O-demethylase (DmtS) activity with respect to the corresponding microorganism. In some such versions, the recombinant microorganism exhibits increased accumulation of 2-pyrone-4,6-dicarboxylic acid (PDC) with respect to the corresponding microorganism.

In some versions, the modification that decreases PDC hydrolase (LigI) activity comprises a modification to a gene encoding a PDC hydrolase (LigI) comprising a sequence having at least 80%, at least 85%, at least 90%, at least 95%, or at least 99% sequence identity to SEQ ID NO:16.

In some versions, the modification that decreases CHMOD methyl esterase (DesC) activity a modification to a gene encoding a CHMOD methyl esterase (DesC) comprising a sequence having at least 80%, at least 85%, at least 90%, at least 95%, or at least 99% sequence identity to SEQ ID NO:18.

In some versions, the modification that decreases CHMOD cis-trans isomerase (DesD) activity comprises a modification to a gene encoding a CHMOD cis-trans isomerase (DesD) comprising a sequence having at least 80%, at least 85%, at least 90%, at least 95%, or at least 99% sequence identity to SEQ ID NO:20.

In some versions, the modification that decreases vanillate/3-O-methylgallate O-demethylase (DmtS) activity comprises a modification to a gene encoding a vanillate/3-O-methylgallate O-demethylase (DmtS) comprising a sequence having at least 80%, at least 85%, at least 90%, at least 95%, or at least 99% sequence identity to SEQ ID NO:22.

In some versions, the recombinant microorganism is from the genus Novosphingobium.

In some versions, the recombinant microorganism is Novosphingobium aromaticivorans.

Another aspect of the invention is directed to methods of producing a compound.

In some versions, the methods comprise culturing a recombinant microorganism of the invention in a medium comprising a plant-derived phenolic.

In some versions, the compound comprises any one or more of coenzyme Q₁₀ (CoQ₁₀), phytoene, lycopene, beta-carotene, zeaxanthin, adonixanthin, astaxanthin, and 2-pyrone-4,6-dicarboxylic acid (PDC).

In some versions, the medium comprises a plant-derived phenolic selected from the group consisting of a syringyl phenolic, a guaiacyl phenolic, and a p-hydroxyphenyl phenolic.

In some versions, the medium comprises depolymerized lignin.

In some versions, the methods comprise further comprising isolating compound from the recombinant microorganism.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Predicted carotenoid biosynthetic pathway based on the annotated genome of N. aromaticivorans. Reactions predicted to be present in wild-type N. aromaticivorans (13, 23) are shown as full arrows and include the genes predicted to code for the enzymes involved. Reactions predicted to occur in strains in which crtG has been replaced in the genome with a crtW gene are shown as dashed arrows. Abbreviations are: DMAPP=dimethylallyl pyrophosphate, IPP=isopentenyl diphosphate, GPP=geranyl diphosphate, FPP=farnesyl diphosphate, and GGPP=geranylgeranyl pyrophosphate.

FIG. 2. HPLC analysis and absorbance spectra of carotenoid and CoQ₁₀ standards used in this study. Data are shown in milliabsorbance units (mAu).

FIGS. 3A-3G. HPLC analyses of acetone:methanol extracts of representative N. aromaticivorans cultures. Absorbances are shown for 273 nm (the wavelength of maximum absorbance for CoQ₁₀; blue line) and 470 nm (a wavelength where all carotenoids investigated have some absorbance; orange line). Standard compounds (top panels in FIGS. 3A-3G) analyzed are astaxanthin, zeaxanthin, β-carotene, lycopene, and CoQ₁₀; vertical dotted lines in the bottom panels of FIGS. 3A-3G show the time of maximum absorbance for the carotenoid standards. Strains analyzed are: 12444Δ1879 (FIG. 3A), 12444ΔcrtB (FIG. 3B), 12444ΔcrtY (FIG. 3C), 12444ΔcrtG (FIG. 3D), 12444ΔcrtGZ (FIG. 3E), 12444SastaW (FIG. 3F), and 12444StaxiW (FIG. 3G). Data are shown as milliabsorbance units (mAu). Major carotenoid peaks are numbered from 1 to 13. Absorbance spectra for all major peaks are shown in FIGS. 2 and 4A-10B.

FIGS. 4A-4D. Compounds detected in HPLC analyses of N. aromaticivorans 12444Δ1879 acetone:methanol extracts. HPLC data reproduced from FIG. 3A (FIG. 4A, top panel) is used to organize the absorbance spectra of different compounds (FIG. 4A, bottom panels), some of which have associated mass spectrometry scans (FIG. 4B, compound 1 (nostoxanthin); FIG. 4C, compound 2; FIG. 4D, compound 3 (caloxanthin)). Dotted lines in the top panel of FIG. 4A denote retention times of carotenoid standards. The mass spectrometry scans in FIGS. 4B-4D are continued from the top panel to the second panel, with each panel including an overlapping section of the scan.

FIG. 5. Compounds detected in HPLC analyses of N. aromaticivorans 12444ΔcrtB acetone:methanol extracts. HPLC data reproduced from FIG. 3B (FIG. 5, top panel) is used to organize the absorbance spectra of different compounds (FIG. 5, bottom panel).

FIG. 6. Compounds detected in HPLC analyses of N. aromaticivorans 12444ΔcrtY acetone:methanol extracts. HPLC data reproduced from FIG. 3C (FIG. 6, top panel) is used to organize the absorbance spectra of different compounds (FIG. 6, bottom panels). Dotted lines in the top panel of FIG. 6 denote retention times of carotenoid standards.

FIG. 7. Compounds detected in HPLC analyses of N. aromaticivorans 12444ΔcrtG acetone:methanol extracts. HPLC data reproduced from FIG. 3D (FIG. 7, top panel) is used to organize the absorbance spectra of different compounds (FIG. 7, bottom panels). Dotted lines in the top panel of FIG. 7 denote retention times of carotenoid standards.

FIG. 8. Compounds detected in HPLC analyses of N. aromaticivorans 12444ΔcrtGZ acetone:methanol extracts. HPLC data reproduced from FIG. 3E (FIG. 8, top panel) is used to organize the absorbance spectra of different compounds (FIG. 8, bottom panels). Dotted lines in the top panel of FIG. 8 denote retention times of carotenoid standards.

FIGS. 9A and 9B. Compounds detected in HPLC analyses of N. aromaticivorans 12444SastaW acetone:methanol extracts. HPLC data reproduced from FIG. 3F (FIG. 9A, top panel) is used to organize the absorbance spectra of different compounds (FIG. 9A, bottom panels), some of which have associated mass spectrometry scans (FIG. 9B). Compound 8's absorbance was too low to obtain an accurate absorbance or mass spectrum. Dotted lines in the top panel of FIG. 9A denote retention times of carotenoid standards. The mass spectrometry scan in FIG. 9B is continued from the top panel to the second panel, with each panel including an overlapping section of the scan.

FIGS. 10A and 10B. Compounds detected in HPLC analyses of N. aromaticivorans 12444StaxiW acetone:methanol extracts. HPLC data reproduced from FIG. 3G (FIG. 10A, top panel) is used to organize the absorbance spectra of different compounds (FIG. 10A, bottom panels), some of which have associated mass spectrometry scans (FIG. 10B). Dotted lines in the top panel of FIG. 10A denote retention times of carotenoid standards. The mass spectrometry scan in FIG. 10B is continued from the top panel to the second panel, with each panel including an overlapping section of the scan.

FIG. 11. SMB-Glucose plates of parent and mutant N. aromaticivorans strains showing the different colony colors in carotenoid pathway deletion mutants (A, left plate) or in crtW-expressing engineered strains (B, right plate). Some colony colors appear similar despite accumulating different carotenoids (FIGS. 3A-G) due to the known similarity in the absorption spectra of their carotenoids (FIGS. 2 and 4A-10B).

FIG. 12. Carotenoid and CoQ₁₀ levels in N. aromaticivorans strains. Filled circles show data for roux bottle vanillate-fed cultures bubbled with a gas mixture of defined composition; open squares show data for shake flask Sorghum APL cultures supplied with atmospheric O₂. Amounts of select carotenoids, normalized by dry cell weight (dcw), are plotted for N. aromaticivorans strains 12444Δ1879 producing nostoxanthin (A), 12444ΔcrtY producing lycopene (B), 12444ΔcrtG producing zeaxanthin (C), 12444ΔcrtGZ producing β-carotene (D), and 12444SastaW producing astaxanthin (E). Data for roux bottle vanillate cultures were plotted against headspace O₂ concentration, and p-values are shown for single-factor ANOVA tests (α=0.05) comparing the data at all three O₂ concentrations for each strain (null hypothesis assuming no difference at the three O₂ concentrations). CoQ₁₀ levels are also shown for those same strains, plus 12444ΔcrtB, grown in roux bottles with vanillate and in shake flasks with Sorghum APL (F). Nostoxanthin accumulation is reported as HPLC peak area due to the lack of a standard. For all other compounds, amounts are reported as mass normalized to dry cell weight. An asterisk (*) denotes data from shake flask Sorghum APL cultures that are significantly different from 21% O₂ roux bottle culture data (Student's T-test, p<0.05).

FIG. 13. Chemical oxygen demand (COD) of Sorghum APL before and after incubation with indicated N. aromaticivorans strains. All detected compounds in Table 2 are included in the graphs, but many are too low in abundance to be visible in the figure. COD provides an estimate of the available chemical energy in a solution by measuring how much oxygen is required to fully oxidize the organic molecules to CO₂. COD was measured as described herein.

FIG. 14. Carotenoid (A-C), CoQ₁₀ (D), and PDC (E) levels in N. aromaticivorans PDC strains grown in Sorghum APL. Filled circles show data for PDC-producing strains, open squares show data for non-PDC producing strains grown in Sorghum APL reproduced from FIG. 12. Amounts of carotenoids and are reported as mass normalized to dry cell weight (dew). PDC levels are reported as concentration measured in the media. An asterisk (*) denotes PDC strain data that are significantly different from the 12444 parent strain data (Student's T-test, p<0.05).

FIG. 15. Dry cell weights normalized to 10 mL of culture of N. aromaticivorans strains grown in SMB+4 mM vanillate at different O₂ concentrations (A) or in Sorghum APL in shake flasks (B).

FIG. 16. Mobile phase binary gradient for HPLC analysis of compounds in N. aromaticivorans acetone:methanol extracts. Solvent A is 70% acetonitrile/30% water and Solvent B is 70% acetonitrile/30% isopropanol. The method proceeds for 63 min.

FIG. 17. Mobile phase binary gradient for HPLC analysis of aromatic compounds in Sorghum APL. Solvent A is 0.2% formic acid in water and solvent B is methanol. The method ends at 10 min.

### DETAILED DESCRIPTION OF THE INVENTION

One aspect of the invention is directed to recombinant microorganisms. The recombinant microorganisms of the invention can be configured for enhanced production of various carotenoids and/or other compounds. The recombinant microorganisms of the invention comprise one or more modifications that increase the activity of one or more genes or gene products, decrease the activity of one or more genes or gene products, or increase the activity of one or more genes or gene products and decrease the activity of one or more genes or gene products. The recombinant microorganisms with the modifications can exhibit enhanced production of a compound such as coenzyme Q₁₀ (CoQ₁₀), phytoene, lycopene, beta-carotene, zeaxanthin, adonixanthin, astaxanthin, and/or 2-pyrone-4,6-dicarboxylic acid (PDC) with respect to corresponding microorganisms not comprising the modifications.

“Modifications that increase the activity of one or more genes or gene products” refers to any modification to microorganism that increases expression of a gene in producing its gene product or increases the functioning of the gene product. “Increase” in this context encompasses increasing beyond a baseline activity. The baseline activity can be a positive baseline activity or null activity. Exemplary modifications that increase the activity of one or more genes or gene products include genetic modifications. The genetic modifications include genetic modifications to a gene in a manner that increases expression of the gene in producing the gene product. Such modifications include operationally connecting the coding sequence to a stronger promoter or enhancer, etc., and/or introducing additional copies of the gene (whether the native gene or a recombinant version). The genetic modifications also include mutations to a first gene (such as a transcription factor or an inhibitor of a transcription factor) that affects the expression of a second gene. The genetic modifications also include one or more copies of an exogenous or heterologous gene introduced into the microorganism. Other genetic modifications are described herein.

“Modifications that decrease the activity of one or more genes or gene products” refers to any modification to a microorganism that decreases expression of the gene and thus production of the gene product and/or decreases the functioning of the gene product per se. “Decrease” in this context encompasses reducing below a positive baseline level of expression and/or activity to a lower level of expression and/or activity. The lower level of expression and/or activity can be a lower positive level of expression and/or activity or null expression and/or activity. Decreasing the functioning of a gene product may comprise decreasing the specific activity of a gene product. Exemplary modifications that decrease the activity of one or more genes or gene products include genetic modifications. Exemplary genetic modifications include mutations to a gene that decrease expression of the gene in producing the gene product. Such mutations may include mutations to the coding sequence, the promoter, an enhancer, any other part of the gene, or the entire gene. Other exemplary genetic modifications include mutations to the coding sequence of a gene that decrease the functioning of a gene product expressed from the gene. Exemplary mutations include substitutions, insertions, and deletions, including partial and full deletions of a particular gene. Other exemplary genetic modifications include recombinant nucleotide sequences configured to express antisense RNAs or other molecules that decrease production of a gene product. Other exemplary genetic modifications include mutations to a first gene (such as a transcription factor or an inhibitor of a transcription factor) that affects the expression of a second gene. Other exemplary genetic modifications are described elsewhere herein. Other modifications include epigenetic modifications, such as methylation, etc.

“Corresponding microorganism” refers to a microorganism of the same species having the same or substantially same genetic and proteomic composition as a recombinant microorganism of the invention, with the exception of genetic and proteomic differences resulting from the modifications specified herein for the recombinant microorganisms of the invention in a given particular embodiment. In some versions, the corresponding microorganism is the native version of the recombinant microorganism of the invention, i.e., the unmodified microorganism as found in nature. The terms “microorganism” and “microbe” are used interchangeably herein.

In some versions, the recombinant microorganisms comprise one or more modifications with respect to a corresponding microorganism not comprising the one or more modifications. The one or more modifications can comprise, in any combination, a modification that decreases phytoene synthase (CrtB) activity with respect to the corresponding microorganism, a modification that decreases lycopene-forming phytoene desaturase (CrtI) activity with respect to the corresponding microorganism, a modification that decreases lycopene cyclase (CrtY) activity with respect to the corresponding microorganism, a modification that decreases beta-carotene hydroxylase (CrtZ) activity with respect to the corresponding microorganism, a modification that decreases 2,2′-beta hydroxylase (CrtG) activity with respect to the corresponding microorganism, and a modification that increases beta-carotene ketolase (CrtW) activity with respect to the corresponding microorganism.

Phytoene synthase (CrtB) activity includes activity characterized by EC 2.5.1.32 and comprises the ability to catalyze the conversion of geranylgeranyl pyrophosphate to phytoene. An exemplary phytoene synthase is CrtB (Saro_1814) of Novosphingobium aromaticivorans, the nucleic acid coding sequence of which is SEQ ID NO:1 and the protein sequence of which is SEQ ID NO:2. Other exemplary phytoene synthases include proteins with phytoene synthase activity having a sequence at least 80%, at least 85%, at least 90%, at least 95%, or at least 99% identical to SEQ ID NO:2. In some versions of the invention, the one or more modifications in the recombinant microorganisms can comprise a genetic modification that decreases phytoene synthase activity. A genetic modification that decreases phytoene synthase activity can comprise a genetic modification to a phytoene synthase gene. A genetic modification to a phytoene synthase gene can comprise a substitution or insertion in or a complete or partial deletion of the phytoene synthase gene.

Lycopene-forming phytoene desaturase (CrtI) activity includes activity characterized by EC 1.3.99.31 and comprises the ability to catalyze the conversion of phytoene to lycopene. An exemplary lycopene-forming phytoene desaturase is CrtI (Saro_1816) of Novosphingobium aromaticivorans, the nucleic acid coding sequence of which is SEQ ID NO:3 and the protein sequence of which is SEQ ID NO:4. Other exemplary lycopene-forming phytoene desaturases include proteins with lycopene-forming phytoene desaturase activity having a sequence at least 80%, at least 85%, at least 90%, at least 95%, or at least 99% identical to SEQ ID NO:4. In some versions of the invention, the one or more modifications in the recombinant microorganisms can comprise a genetic modification that decreases lycopene-forming phytoene desaturase activity. A genetic modification that decreases lycopene-forming phytoene desaturase activity can comprise a genetic modification to a lycopene-forming phytoene desaturase gene. A genetic modification to a lycopene-forming phytoene desaturase gene can comprise a substitution or insertion in or a complete or partial deletion of the lycopene-forming phytoene desaturase gene.

Lycopene cyclase (CrtY) activity includes activity characterized by EC 5.5.1.19 and comprises the ability to catalyze the conversion of lycopene to beta-carotene. An exemplary lycopene cyclase is CrtY (Saro_1817) of Novosphingobium aromaticivorans, the nucleic acid coding sequence of which is SEQ ID NO5; and the protein sequence of which is SEQ ID NO:6. Other exemplary lycopene cyclases include proteins with lycopene cyclase activity having a sequence at least 80%, at least 85%, at least 90%, at least 95%, or at least 99% identical to SEQ ID NO:6. In some versions of the invention, the one or more modifications in the recombinant microorganisms can comprise a genetic modification that decreases lycopene cyclase activity. A genetic modification that decreases lycopene cyclase activity can comprise a genetic modification to a lycopene cyclase gene. A genetic modification to a lycopene cyclase gene can comprise a substitution or insertion in or a complete or partial deletion of the lycopene cyclase gene.

Beta-carotene hydroxylase (CrtZ) activity includes activity characterized by EC 1.14.15.24 and comprises the ability to catalyze the conversion of beta-carotene to zeaxanthin, among other activities. An exemplary beta-carotene hydroxylase is CrtZ (Saro_1168) of Novosphingobium aromaticivorans, the nucleic acid coding sequence of which is SEQ ID NO:7 and the protein sequence of which is SEQ ID NO:8. Other exemplary beta-carotene hydroxylases include proteins with beta-carotene hydroxylase activity having a sequence at least 80%, at least 85%, at least 90%, at least 95%, or at least 99% identical to SEQ ID NO:8. In some versions of the invention, the one or more modifications in the recombinant microorganisms can comprise a genetic modification that decreases beta-carotene hydroxylase activity. A genetic modification that decreases beta-carotene hydroxylase activity can comprise a genetic modification to a beta-carotene hydroxylase gene. A genetic modification to a beta-carotene hydroxylase gene can comprise a substitution or insertion in or a complete or partial deletion of the beta-carotene hydroxylase gene.

2,2′-Beta hydroxylase (CrtG) activity includes activity characterized by EC 1.14.13.—and comprises the ability to catalyze the conversion of zeaxanthin to caloxanthin, caloxanthin to nostoxanthin, and/or beta-carotene to 2,2′-dihydroxy-beta-carotene. An exemplary 2,2′-beta hydroxylase is CrtG (Saro_0236) of Novosphingobium aromaticivorans, the nucleic acid coding sequence of which is SEQ ID NO:9 and the protein sequence of which is SEQ ID NO:10. Other exemplary 2,2′-beta hydroxylases include proteins with 2,2′-beta hydroxylase activity having a sequence at least 80%, at least 85%, at least 90%, at least 95%, or at least 99% identical to SEQ ID NO:10. In some versions of the invention, the one or more modifications in the recombinant microorganisms can comprise a genetic modification that decreases 2,2′-beta hydroxylase activity. A genetic modification that decreases 2,2′-beta hydroxylase activity can comprise a genetic modification to a 2,2′-beta hydroxylase gene. A genetic modification to a 2,2′-beta hydroxylase gene can comprise a substitution or insertion in or a complete or partial deletion of the 2,2′-beta hydroxylase gene.

Beta-carotene ketolase activity includes activity characterized by EC 1.14.99.63 and comprises the ability to catalyze the conversion of beta-carotene to canthaxanthin, and/or the conversion of zeaxanthin to adonixanthin, and/or the conversion of adonixanthin to astaxanthin. Beta-carotene ketolase activity is performed by beta-carotene ketolase, sometimes referred to as CrtW. An exemplary beta-carotene ketolase is CrtW of Sphingomonas taxi, the nucleic acid coding sequence of which is SEQ ID NO:11 and the protein sequence of which is SEQ ID NO:12. Another exemplary beta-carotene ketolase is CrtW of Sphingomonas astaxanthinfaciens, the nucleic acid coding sequence of which is SEQ ID NO:13 and the protein sequence of which is SEQ ID NO: 14. Other exemplary beta-carotene ketolases include proteins with beta-carotene ketolase activity having a sequence at least 80%, at least 85%, at least 90%, at least 95%, or at least 99% identical to SEQ ID NOS:12 or 14. Other beta-carotene ketolases are known in the art. In some versions of the invention, the one or more modifications in the recombinant microorganisms can comprise a genetic modification that increases beta-carotene ketolase activity. A genetic modification that increases beta-carotene ketolase activity can comprise a recombinant beta-carotene ketolase gene. In some versions, the recombinant beta-carotene ketolase gene is an exogenous recombinant beta-carotene ketolase gene newly introduced to the microorganism. In some versions, the recombinant beta-carotene ketolase gene is a modified form of an endogenous beta-carotene ketolase gene already present in the microorganism.

2-Pyrone-4,6-dicarboxylic acid (PDC) hydrolase activity (LigI) activity comprises activity characterized by EC 3.1.1.57 and comprises the ability to hydrolyze PDC to produce 4-oxalomesaconate (OMA). An exemplary PDC hydrolase is LigI (Saro_2819) of Novosphingobium aromaticivorans, the nucleic acid coding sequence of which is SEQ ID NO15; and the protein sequence of which is SEQ ID NO:16. Other exemplary PDC hydrolases include proteins with PDC hydrolase activity having a sequence at least 80%, at least 85%, at least 90%, at least 95%, or at least 99% identical to SEQ ID NO: 16. In some versions of the invention, the one or more modifications in the recombinant microorganisms can comprise a genetic modification that decreases PDC hydrolase activity. A genetic modification that decreases PDC hydrolase activity can comprise a genetic modification to a PDC hydrolase gene. A genetic modification to a PDC hydrolase gene can comprise a substitution or insertion in or a complete or partial deletion of the PDC hydrolase gene.

4-Carboxy-2-hydroxy-6-methoxy-6-oxohexa-2,4-dienoate (CHMOD) methyl esterase (DesC) activity comprises the ability to demethylate CHMOD to produce OMA. An exemplary CHMOD methyl esterase is desC/DesC (Saro_2864) of Novosphingobium aromaticivorans, the nucleic acid coding sequence of which is SEQ ID NO: 17 and the protein sequence of which is SEQ ID NO:18. Other exemplary CHMOD methyl esterases include proteins with CHMOD methyl esterase activity having a sequence at least 80%, at least 85%, at least 90%, at least 95%, or at least 99% identical to SEQ ID NO:18. In some versions of the invention, the one or more modifications in the recombinant microorganisms can comprise a genetic modification that decreases CHMOD methyl esterase activity. A genetic modification that decreases CHMOD methyl esterase activity can comprise a genetic modification to a CHMOD methyl esterase gene. A genetic modification to a CHMOD methyl esterase gene can comprise a substitution or insertion in or a complete or partial deletion of the CHMOD methyl esterase gene.

4-Carboxy-2-hydroxy-6-methoxy-6-oxohexa-2,4-dienoate (CHMOD) cis-trans isomerase (DesD) activity comprises the ability to isomerize stereoisomers of CHMOD. An exemplary CHMOD cis-trans isomerase is DesD (Saro_2865) of Novosphingobium aromaticivorans, the nucleic acid coding sequence of which is SEQ ID NO: 19 and the protein sequence of which is SEQ ID NO:20. Other exemplary CHMOD cis-trans isomerases include proteins with CHMOD cis-trans isomerase activity having a sequence at least 80%, at least 85%, at least 90%, at least 95%, or at least 99% identical to SEQ ID NO:20. In some versions of the invention, the one or more modifications in the recombinant microorganisms can comprise a genetic modification that decreases CHMOD cis-trans isomerase activity. A genetic modification that decreases CHMOD cis-trans isomerase activity can comprise a genetic modification to a CHMOD cis-trans isomerase gene. A genetic modification to a CHMOD cis-trans isomerase gene can comprise a substitution or insertion in or a complete or partial deletion of the CHMOD cis-trans isomerase gene.

Vanillate/3-O-methylgallate O-demethylase (DmtS) activity comprises the ability to 0-demethylate substrates such as vanillate and/or 3-methoxygallic acid. Vanillate/3-O-methylgallate O-demethylases include enzymes having activity characterized under one more of Enzyme Commission (EC) Numbers 2.1.1.341 and 1.14.13.82. An exemplary vanillate/3-O-methylgallate O-demethylase is VanA or DmtS (Saro_2861) of Novosphingobium aromaticivorans, the nucleic acid coding sequence of which is SEQ ID NO:21 and the protein sequence of which is SEQ ID NO:22. Other exemplary vanillate/3-O-methylgallate O-demethylases include proteins with phytoene synthase activity having a sequence at least 80%, at least 85%, at least 90%, at least 95%, or at least 99% identical to SEQ ID NO:22. In some versions of the invention, the one or more modifications in the recombinant microorganisms can comprise a genetic modification that decreases vanillate/3-O-methylgallate O-demethylase activity. A genetic modification that decreases vanillate/3-O-methylgallate O-demethylase activity can comprise a genetic modification to a vanillate/3-O-methylgallate O-demethylase gene. A genetic modification to a vanillate/3-O-methylgallate O-demethylase gene can comprise a substitution or insertion in or a complete or partial deletion of the vanillate/3-O-methylgallate O-demethylase gene.

“Gene” refers to a nucleic acid sequence capable of producing a gene product and may include such genetic elements as a coding sequence together with any other genetic elements required for transcription and/or translation of the coding sequence. Such genetic elements may include a promoter, an enhancer, and/or a ribosome binding site (RBS), among others. In some versions, multiple genes are configured in an operon, in which multiple coding sequences are operationally connected to a single promoter. Each coding sequence and promoter pair in such instances are considered herein to constitute separate genes, despite comprising the same promoter.

“Gene product” refers to products such as a polypeptide or an mRNA encoded and produced by a particular gene.

“Operationally connected” refers to a relationship between two genetic elements (e.g., a promoter and coding sequence), in which one of the genetic elements controls or affects the activity of the other genetic element.

“Endogenous” used in reference to a genetic element means that the genetic element is native to the microorganism in which it is disposed.

“Exogenous” used in reference to a genetic element means that the genetic element is not native to the microorganism in which it is disposed.

“Heterologous” used in reference to a genetic element means that the genetic element is derived from a different species than that in which it is disposed or is disposed in relation to another element (genetic element, sequence) in a non-natural arrangement.

“Recombinant” as used herein with reference to nucleic acid molecules or polypeptides refers to nucleic acid molecules or polypeptides having a non-natural nucleic acid or polypeptide sequence, respectively. Recombinant” as used herein with reference to a gene refers to a gene having a non-natural nucleic acid sequence, is exogenous, is heterologous, or is endogenous to a given microbe but is disposed within the microbe (e.g., within the microbe's genome) at a locus different from the native form of the gene. “Recombinant” as used herein with reference to a cell or microorganism refers to a cell or microorganism that contains a recombinant nucleic acid molecule, polypeptide, or gene.

“Genetic modification” as used herein refers to any difference in the nucleic acid composition of a cell with respect to a corresponding cell, whether in the cell's native chromosome or in endogenous or exogenous non-chromosomal plasmids harbored within the cell.

“Overexpress” as used herein means that a particular gene product is produced at a higher level in one cell, such as a recombinant cell, than in a corresponding cell. For example, a microorganism that includes a recombinant nucleic acid configured to overexpress a gene product produces the gene product at a greater amount than a microorganism of the same species that does not include the recombinant nucleic acid.

A “homologous” gene or protein is a gene or protein inherited in two species from a common ancestor. While homologous genes or proteins can be similar in sequence, similar sequences are not necessarily homologous.

The terms “identical” or “percent identity”, in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described herein (or other algorithms available to persons of skill) or by visual inspection. For sequence comparison and identity determination, one sequence typically acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence based on the designated program parameters. A typical reference sequence of the invention is any nucleic acid or amino acid sequence described herein. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)). One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915). In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in determining sequence identity of sequences described herein.

In addition to mechanisms described elsewhere herein, genetic modifications for increasing the activity of a gene or protein include but are not limited to placing the coding sequence under the control of a more active promoter, increasing the copy number of genes comprising the coding sequence, introducing a translational enhancer on a gene comprising the coding sequence (see, e.g., Olins et al. Journal of Biological Chemistry, 1989, 264(29):16973-16976), and/or modifying factors (e.g., transcription factors or genes therefor) that control expression of a gene comprising the coding sequence. Increasing the copy number of genes comprising a coding sequence can be performed by introducing one or more additional copies of the native gene to the microorganism, introducing one or more heterologous homologs to the microorganism, introducing one or more copies of recombinant versions of the native gene or heterologous homolog to the microorganism, etc. Genes expressing a given coding sequence may be incorporated into the microbial genome or included on an extrachromosomal genetic construct such as a plasmid.

In addition to mechanisms described elsewhere herein, genetic modifications for decreasing the activity of a gene or protein include but are not limited to substitutions, partial or complete deletions, insertions, or other variations to a coding sequence or a sequence controlling the transcription or translation of a coding sequence, such as placing a coding sequence under the control of a less active promoter, etc. In some versions, the genetic modifications can include the introduction of constructs that express ribozymes or antisense sequences that target the mRNA of the gene of interest. Various other genetic modifications that decrease the activity of a gene or gene product are described elsewhere herein.

Various methods for introducing genetic modifications are well known in the art and include homologous recombination, among other mechanisms. See, e.g., Green et al., Molecular Cloning: A laboratory manual, 4^{th} ed., Cold Spring Harbor Laboratory Press (2012) and Sambrook et al., Molecular Cloning: A Laboratory Manual, 3^{rd} ed., Cold Spring Harbor Laboratory Press (2001).

The recombinant genes of the invention can be codon-optimized for the particular microorganism in which they are introduced. Codon optimization can be performed for any nucleic acid by a number of programs, including “GENEGPS”-brand expression optimization algorithm by DNA 2.0 (Menlo Park, CA), “GENEOPTIMIZER”-brand gene optimization software by Life Technologies (Grand Island, NY), and “OPTIMUMGENE”-brand gene design system by GenScript (Piscataway, NJ). Other codon optimization programs or services are well known and commercially available.

The recombinant microorganisms of the invention may be prokaryotic or eukaryotic. Suitable prokaryotes include bacteria and archaea. Suitable types of bacteria include α- and γ-proteobacteria, gram-positive bacteria, gram-negative bacteria, ungrouped bacteria, phototrophs, lithotrophs, and organotrophs. Suitable eukaryotes include yeast and other fungi. The microorganism in some versions can be from an order selected from the group consisting of Sphingomonadales and Pseudomonadales. The microorganism in some versions can be from a family selected from the group consisting of Sphingomonadaceae, Pseudomonadaceae, and Enterobacteriaceae. The microorganism in some versions can be from a genus selected from the group consisting of Sphingomonas, Sphingobium, Sphingosinicella, Sphingopyxis, Novosphingobium, Pseudomonas, Erythrobacter (e.g., sp. SG61-1L), Altererythrobacter, Enterobacter, and Klebsiella, among others.

The microorganism in some versions can be a phenol-degrading microorganism, such as a phenol-degrading bacterium. Phenol-degrading microorganisms, including phenol-degrading bacteria, are well known in the art. See, e.g., Gu et al. 2016 (Gu Q, Wu Q, Zhang J, Guo W, Wu H, Sun M. Community Analysis and Recovery of Phenol-degrading Bacteria from Drinking Water Biofilters. Front Microbiol. 2016 Apr. 12; 7:495), Ramió-Pujol et al. 2013 (Ramió-Pujol S, Baneras L, Artigas J, Romani A M. Changes of the phenol-degrading bacterial community during the decomposition of submersed Platanus acerifolia leaves. FEMS Microbiol Lett. 2013 January; 338(2):184-91), Bastos et al. 2000 (Bastos A E, Moon D H, Rossi A, Trevors J T, Tsai S M. Salt-tolerant phenol-degrading microorganisms isolated from Amazonian soil samples. Arch Microbiol. 2000 November; 174(5):346-52), van Schie et al. 1998 (van Schie P M, Young L Y. Isolation and characterization of phenol-degrading denitrifying bacteria. Appl Environ Microbiol. 1998 July; 64(7):2432-8), Paisio et al. 2012 (Paisio C E, Talano M A, González P S, Busto V D, Talou J R, Agostini E. Isolation and characterization of a Rhodococcus strain with phenol-degrading ability and its potential use for tannery effluent biotreatment. Environ Sci Pollut Res Int. 2012 September; 19(8):3430-9), Kumari et al. 2013 (Kumari S, Chetty D, Ramdhani N, Bux F. Phenol degrading ability of Rhodococcus pyrinidivorans and Pseudomonas aeruginosa isolated from activated sludge plants in South Africa. J Environ Sci Health A Tox Hazard Subst Environ Eng. 2013; 48(8):947-53), among others. Examples of phenol-degrading microorganisms include Pseudomonas putida (Abu Hamed T., Bayraktar E., Mehmetoǧlu Ü., Mehmetoǧlu T. (2004). The biodegradation of benzene, toluene and phenol in a two-phase system. Biochem. Eng. J. 19 137-146), Gliomastix indicus (Singh R. K., Kumar S., Kumar S., Kumar A. (2008) Biodegradation kinetic studies for the removal of p-cresol from wastewater using Gliomastix indicus MTCC 3869. Biochem. Eng. J. 40 293-303), Sphingomonas chlorophenolica (Nair C. I., Jayachandran K., Shashidhar S. (2008). Biodegradation of phenol. Afr. J. Biotechnol. 7 4951-4958), Bacillus brevis (Arutchelvan V., Kanakasabai V., Elangovan R., Nagarajan S., Muralikrishnan V. (2006). Kinetics of high strength phenol degradation using Bacillus brevis. J. Hazardous Materials 129 216-222), and Cyanobacterium synechococcus (Song H., Liu Y., Xu W., Zeng G., Aibibu N., Xu L., et al. (2009). Simultaneous Cr (VI) reduction and phenol degradation in pure cultures of Pseudomonas aeruginosa CCTCC AB91095. Bioresour. Technol. 100 5079-5084), and Acinetobacter sp. (Gu Q, Wu Q, Zhang J, Guo W, Wu H, Sun M. Community Analysis and Recovery of Phenol-degrading Bacteria from Drinking Water Biofilters. Front Microbiol. 2016 Apr. 12; 7:495). Other examples of phenol-degrading microorganisms include Achromobacter sp., Alcaligenes denitripzcans, Arthrobacter sp., Arthrobacter sulphureus, Acidovorax delafieldii, Bacillus cereus, Brevibacterium sp., Burkholderia sp., Burkholderia cepacia, Burkholderia cocovenenans, Burkholderia xenovorans, Chryseobacterium sp., Cycloclasticus sp., Janibacter sp., Marinobacter, Mycobacterium sp., Mycobacterium flavescens, Mycobacterium vanbaalenii, Mycobacterium sp., Nocardioides aromaticivorans, Pasteurella sp., Polaromonas naphthalenivorans, Pseudomonas sp., Pseudomonas paucimobilis, Pseudomonas vesicularis, Pseudomonas putida, Pseudomonas fluorescens, Pseudomonas stutzeri, Pseudomonas saccharophilia, Ralstonia sp., Rhodococcus sp., Rhodococcus erythropolis, Staphylococcus sp., Stenotrophomonas maltophilia, Sphingomonas yanoikuyae, Sphingomonas sp., Sphingomonas paucimobilis, Sphingomonas wittichii, Terrabacter sp., and Xanthamonas sp. (Seo J-S, Keum Y-S, Li Q X. Bacterial Degradation of Aromatic Compounds. International Journal of Environmental Research and Public Health. 2009; 6(1):278-309.) Other examples of phenol-degrading microorganism include Acinetobacter calcoaceticus, Rhodococcus aetherivorans, Rhodococcus ruber SD3, Aspergillus oryzae, and Aspergillus flavus (Xu N, Qiu C, Yang Q, Zhang Y, Wang M, Ye C, Guo M. Analysis of Phenol Biodegradation in Antibiotic and Heavy Metal Resistant Acinetobacter lwoffii NL 1. Front Microbiol. 2021 Sep. 10; 12:725755), among others.

An exemplary microorganism from the genus Novosphingobium is Novosphingobium aromaticivorans. Novosphingobium aromaticivorans DSM12444 can naturally catabolize multiple aromatic compounds containing H, G, and S units via protocatechuic acid.

The recombinant microorganisms are preferably configured to exhibit enhanced accumulation of various compounds with respect to a corresponding microorganism. “Accumulation” in this context refers to appearance of the compound intracellularly, extracellularly (e.g., secretion), or both. The enhanced accumulation can be exhibited as an increased amount of the compound at steady state or an increased rate of appearance over time. The recombinant microorganisms in such versions may include any one or more of the modifications described herein, in any combination.

The recombinant microorganisms of the invention preferably exhibit enhanced accumulation of at least one compound with respect to the corresponding microorganism when the recombinant microorganism and the corresponding organism are grown under identical conditions. The accumulation may be enhanced by a factor of at least about 1.1, at least about 1.5, at least about 2, at least about 2.5, at least about 3, at least about 3.5, at least about 4, at least about 4.5, at least about 5, at least about 5.5, at least about 6, or at least about 6.5 and/or up to about 6.5, up to about 7, or more. Such increases may reflect an increase by mass.

The accumulated compounds of the invention can comprise any one or more of coenzyme Q₁₀ (CoQ₁₀), phytoene, lycopene, beta-carotene, zeaxanthin, adonixanthin, astaxanthin, and 2-pyrone-4,6-dicarboxylic acid (PDC).

Recombinant microorganisms configured for enhanced accumulation of CoQ₁₀ can comprise any one or more of a modification that decreases phytoene synthase (CrtB) activity, a modification that decreases lycopene-forming phytoene desaturase (CrtI) activity, a modification that decreases lycopene cyclase (CrtY) activity, a modification that decreases beta-carotene hydroxylase (CrtZ) activity, and/or a modification that decreases 2,2′-beta hydroxylase (CrtG) activity. In some versions, the recombinant microorganisms configured for enhanced accumulation of CoQ₁₀ comprises any one or more of a modification that decreases phytoene synthase (CrtB) activity, a modification that decreases lycopene-forming phytoene desaturase (CrtI) activity, and/or a modification that decreases lycopene cyclase (CrtY) activity. In some versions, the recombinant microorganism configured for enhanced accumulation of CoQ₁₀ comprises a modification that decreases phytoene synthase (CrtB) activity.

Recombinant microorganisms configured for enhanced accumulation of lycopene can comprise a modification that decreases lycopene cyclase (CrtY) activity.

Recombinant microorganisms configured for enhanced accumulation of zeaxanthin can comprise a modification that decreases 2,2′-beta hydroxylase (CrtG) activity.

Recombinant microorganisms configured for enhanced accumulation of beta-carotene can comprise a modification that decreases 2,2′-beta hydroxylase (CrtG) activity, a modification that decreases beta-carotene hydroxylase (CrtZ) activity, or, preferably, both.

Recombinant microorganisms configured for enhanced accumulation of adonixanthin and/or astaxanthan can comprise a modification that decreases 2,2′-beta hydroxylase (CrtG) activity, a modification that increases beta-carotene ketolase (CrtW) activity, or, preferably both.

Recombinant microorganisms configured for enhanced accumulation of PDC can comprise a modification that decreases 2-pyrone-4,6-dicarboxylic acid (PDC) hydrolase (LigI) activity, a modification that decreases 4-carboxy-2-hydroxy-6-methoxy-6-oxohexa-2,4-dienoate (CHMOD) methyl esterase (DesC) activity, a modification that decreases 4-carboxy-2-hydroxy-6-methoxy-6-oxohexa-2,4-dienoate (CHMOD) cis-trans isomerase (DesD) activity, and/or a modification that decreases vanillate/3-O-methylgallate O-demethylase (DmtS) activity or, preferably, each of the foregoing modifications.

The recombinant microorganisms of the invention can accordingly be used to produce one or more of the compounds of the invention. Such methods can comprise culturing the recombinant microorganisms of the invention in a manner such that the compound to be produced accumulates intracellularly, extracellularly or both.

Recombinant microorganisms employed for production of CoQ₁₀ can comprise any one or more of a modification that decreases phytoene synthase (CrtB) activity, a modification that decreases lycopene-forming phytoene desaturase (CrtI) activity, a modification that decreases lycopene cyclase (CrtY) activity, a modification that decreases beta-carotene hydroxylase (CrtZ) activity, and/or a modification that decreases 2,2′-beta hydroxylase (CrtG) activity. In some versions, the recombinant microorganisms employed for production of CoQ₁₀ comprises any one or more of a modification that decreases phytoene synthase (CrtB) activity, a modification that decreases lycopene-forming phytoene desaturase (CrtI) activity, and/or a modification that decreases lycopene cyclase (CrtY) activity. In some versions, the recombinant microorganism employed for production of CoQ₁₀ comprises a modification that decreases phytoene synthase (CrtB) activity.

Recombinant microorganisms employed for production of lycopene can comprise a modification that decreases lycopene cyclase (CrtY) activity.

Recombinant microorganisms employed for production of zeaxanthin can comprise a modification that decreases 2,2′-beta hydroxylase (CrtG) activity.

Recombinant microorganisms employed for production of beta-carotene can comprise a modification that decreases 2,2′-beta hydroxylase (CrtG) activity, a modification that decreases beta-carotene hydroxylase (CrtZ) activity, or, preferably, both.

Recombinant microorganisms employed for production of adonixanthin and/or astaxanthan can comprise a modification that decreases 2,2′-beta hydroxylase (CrtG) activity, a modification that increases beta-carotene ketolase (CrtW) activity, or, preferably both.

Recombinant microorganisms employed for production of PDC can comprise a modification that decreases 2-pyrone-4,6-dicarboxylic acid (PDC) hydrolase (LigI) activity, a modification that decreases 4-carboxy-2-hydroxy-6-methoxy-6-oxohexa-2,4-dienoate (CHMOD) methyl esterase (DesC) activity, a modification that decreases 4-carboxy-2-hydroxy-6-methoxy-6-oxohexa-2,4-dienoate (CHMOD) cis-trans isomerase (DesD) activity, and/or a modification that decreases vanillate/3-O-methylgallate O-demethylase (DmtS) activity or, preferably, each of the foregoing modifications.

In some versions, the compounds of the invention are produced by culturing a recombinant microorganism of the invention in a medium comprising a plant-derived phenolic. The plant-derived phenolic can comprise any of a number of phenolics obtained from processing plant lignocellulosic biomass. Exemplary plant-derived phenolics comprise syringyl phenolics, guaiacyl phenolics, and p-hydroxyphenyl phenolics. Exemplary syringyl phenolics include syringaldehyde, syringic acid, and S-diketone. Exemplary guaiacyl phenolics include vanillin, vanillic acid, and G-diketone. Exemplary hydroxyphenyl phenolics include p-coumaric acid, p-hydroxybenzaldehyde, and p-hydroxybenzoic acid. Other plant-derived phenolics include methyl guaiacol, propyl guaiacol, dihydroconiferyl alcohol, methyl syringol, p-hydroxy benzoic acid methyl ester, dihydrop-hydroxy cinnamic acid methyl ester, dihydrosyringol alcohol, and dihydroferulic acid methyl ester, among others.

The plant-derived phenolic can be derived and/or provided in the form of depolymerized lignin, such as chemically depolymerized lignin. Methods of depolymerizing lignin are well known in the art. See Pandey et al. 2010 (Pandey M P, Kim C S. Lignin Depolymerization and Conversion: A Review of Thermochemical Methods. Chemical & Engineering Technology, 2010, Vol. 34, Issue 1, pp. 3-145) and Wang et al. 2013 (Wang H, Tucker M, Ji Y. Recent Development in Chemical Depolymerization of Lignin: A Review. Journal of Applied Chemistry, 2013, Volume 2013, Article ID 838645).

The depolymerized lignin can be derived from pretreated lignocellulosic biomass. Methods of pretreating lignocellulosic biomass are well known in the art. See Kumar et al. 2017 (Kumar A K and Sharma S. Recent Updates on Different Methods of Pretreatment of Lignocellulosic Feedstocks: A Review. Bioresour. Bioprocess. (2017) 4:7); Kumar et al. 2009 (Kumar, P.; Barrett, D. M.; Delwiche, M. J.; Stroeve, P., Methods for Pretreatment of lignocellulosic Biomass for Efficient Hydrolysis and Biofuel Production. Industrial & Engineering Chemistry Research 2009, 48, (8), 3713-3729); Wang et al. 2013 (Wang H, Tucker M, Ji Y. Recent Development in Chemical Depolymerization of Lignin: A Review. (2013) Journal of Applied Chemistry. 2013:1-9), and Karlen et al. 2020 (Karlen S D, Fasahati P, Mazaheri M, Serate J, Smith R A, Sirobhushanam S, Chen M, Tymkhin V I, Cass C L, Liu S, Padmakshan D, Xie D, Zhang Y, McGee M A, Russell J D, Coon J J, Kaeppler H F, de Leon N, Maravelias C T, Runge T M, Kaeppler S M, Sedbrook J C, Ralph J. Assessing the viability of recovering hydroxycinnamic acids from lignocellulosic biorefinery alkaline pretreatment waste streams. ChemSusChem. 2020 Jan. 26). Examples include chipping, grinding, milling, steam pretreatment, ammonia fiber expansion (AFEX, also referred to as ammonia fiber explosion), ammonia recycle percolation (ARP), CO₂ explosion, steam explosion, ozonolysis, wet oxidation, acid hydrolysis, dilute-acid hydrolysis, alkaline hydrolysis, organosolv, ionic liquids, gamma-valerolactone, and pulsed electrical field treatment, among others.

The lignocellulosic biomass can be derived from any source, such as corn cobs, corn stover, cotton seed hairs, grasses, hardwood stems, leaves, newspaper, nut shells, paper, softwood stems, Sorghum, switchgrass, waste papers from chemical pulps, wheat straw, wood, woody residues, mixed biomass species such as those produced by native prairie, and other sources.

The medium in some versions can additionally or alternatively comprise a fermentable sugar. Non-limiting examples of suitable fermentable sugars include adonitol, arabinose, arabitol, ascorbic acid, chitin, cellubiose, dulcitol, erythrulose, fructose, fucose, galactose, glucose, gluconate, inositol, lactose, lactulose, lyxose, maltitol, maltose, maltotriose, mannitol, mannose, melezitose, melibiose, palatinose, pentaerythritol, raffinose, rhamnose, ribose, sorbitol, sorbose, starch, sucrose, trehalose, xylitol, xylose, and hydrates thereof, among others.

In some versions, the fermentable sugar may be replaced by other organic compounds that support growth of the recombinant microorganism. This includes but is not limited to the other organic compounds that are present in the deconstructed biomass fractions from the crops or plant species mentioned above.

The accumulated compound can be isolated from the recombinant microorganism. An intracellularly accumulated compound can be isolated from the recombinant microorganism by, for example, lysing the recombinant microorganism and purifying the compound from at least some of the lysed components thereof. An extracellularly accumulated compound can be isolated from the recombinant microorganism, for example, by separating the medium in which the recombinant microorganism is cultured from the recombinant microorganism. In some versions, the compound is isolated from the medium.

A recitation herein of a microorganism “comprising” a mutation in or to a particular gene refers to a gene that would be present were it not for the mutation, e.g., the gene present in a corresponding microorganism. Thus, the recitation of a microorganism “comprising” a mutation in or to a particular gene encompasses a mutated form of the gene present in the microorganism, a partially deleted remnant of the gene present in the microorganism, a complete absence of the gene (e.g., as resulting from a complete deletion of the gene) in the microorganism, or other configurations.

For generating recombinant microorganisms suitable for producing PDC, the recombinant microorganisms of the invention can include any of the modifications disclosed or claimed in US 2020/0263215 A1, U.S. Pat. No. 11,028,418 B2, US 2021/0261993 A1, and U.S. Pat. No. 11,981,946 B2, which are incorporated herein by reference in their entireties.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

Production of Carotenoids from Aromatics and Pretreated Lignocellulosic Biomass by Novosphingobium aromaticivorans

### SUMMARY

Carotenoids are lipophilic compounds found in the membranes of various organisms. Individual carotenoids are also commodity chemicals, produced industrially for use as food additives, nutritional supplements, cosmetics, and pharmaceuticals. The alphaproteobacterium Novosphingobium aromaticivorans has previously been established as a potential platform microbe for converting aromatic compounds derived from lignocellulosic plant biomass into valuable extracellular products. Here, we show that N. aromaticivorans DSM 12444 cells naturally produce the carotenoid nostoxanthin, and we construct a set of gene deletion mutants that accumulate β-carotene, lycopene, or zeaxanthin, which are predicted intermediates in nostoxanthin biosynthesis as well as commodity chemicals. We also show that a mutant strain heterologously expressing a CrtW protein accumulates the carotenoid astaxanthin. When grown on vanillate as the carbon source, we find that the levels of carotenoids are not significantly affected by O₂ concentration in the tested range of 5% to 21% O₂. We also show that these carotenoids are produced at comparable levels when strains are grown in liquor from alkaline pretreated Sorghum biomass (Sorghum APL), which contains a mixture of aromatics. Finally, we construct strains that produce zeaxanthin, β-carotene, or astaxanthin concurrently with 2-pyrone-4,6-dicarboxylic acid, a potential building block for biodegradable polymers, when grown in Sorghum APL. Combined, our results show that N. aromaticivorans can simultaneously produce valuable intracellular and extracellular commodities when grown in the presence of either pure aromatics or pretreated lignocellulosic biomass.

There is economic and environmental interest in generating commodity chemicals from renewable resources, such as lignocellulosic biomass, that can substitute for chemicals derived from fossil fuels. The bacterium Novosphingobium aromaticivorans is a promising microbial platform for producing commodity chemicals from lignocellulosic biomass because it can produce these from compounds in pretreated lignocellulosic biomass, which many industrial microbial catalysts cannot metabolize. Here, we show that N. aromaticivorans can be engineered to produce several valuable carotenoids. We also show that engineered N. aromaticivorans strains can produce these lipophilic chemicals concurrently with the extracellular commodity chemical 2-pyrone-4,6-dicarboxylic acid when grown in a complex liquor obtained from alkaline pretreated lignocellulosic biomass. Concurrent microbial production of valuable intra- and extracellular products can increase the economic value generated from conversion of lignocellulosic biomass-derived compounds into commodity chemicals and facilitate separation of water- and membrane-soluble products.

### INTRODUCTION

The aromatic polymer lignin is a major component of lignocellulosic plant biomass and is estimated to represent as much as 30% of the organic carbon in the biosphere (1). However, the heterogeneous structure and chemical composition of lignin have limited its economic value to industry. In addition, the mixture of aromatic compounds that results from lignocellulosic biomass deconstruction is often not metabolized by commonly used industrial microbes. We are interested in developing microbial catalysts that can convert heterogeneous mixtures of biomass-derived compounds, including aromatics, into valuable products.

We and others have been exploring Novosphingobium aromaticivorans, an alphaproteobacterium of the Sphingomonadales order, as a platform for producing valuable compounds (2) because it is amenable to genomic modification (3, 4) and can metabolize many components of deconstructed lignocellulosic biomass, including aromatic monomers (2) and some dimers (3, 5). For example, N. aromaticivorans DSM 12444 has been engineered to stoichiometrically convert the major aromatic monomers in deconstructed plant biomass into 2-pyrone-4,6-dicarboxylic acid (PDC), a potential polyester precursor (6) that is secreted into the media (2, 7). This study sought to expand the suite of valuable compounds that N. aromaticivorans can produce from biomass-derived aromatics.

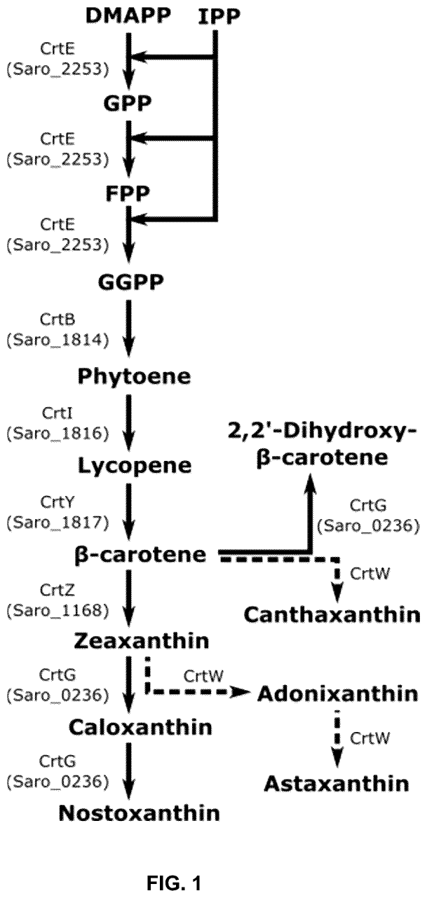

The genome sequence of N. aromaticivorans DSM 12444 predicts that this bacterium can produce the carotenoid nostoxanthin (13). Amongst the intermediates in the predicted nostoxanthin synthesis pathway of N. aromaticivorans are the industrially valuable carotenoids lycopene, β-carotene, and zeaxanthin (FIG. 1). A recent genome-scale metabolic model of N. aromaticivorans suggested that carotenoids could be some of the most profitable products made from plant biomass by N. aromaticivorans because of their high economic value and yields (14).

N. aromaticivorans (when it was known as Sphingomonas aromaticivorans F199) has also been shown to produce the lipophilic Coenzyme Q₁₀ (CoQ₁₀) (15). CoQ₁₀ is also the main isoprenoid quinone in humans and is a commodity chemical used in the pharmaceutical and cosmetics industries (16-19). Currently, bacteria that produce CoQ₁₀ industrially (16, 20) cannot metabolize the aromatics present in deconstructed plant biomass. Thus, there is potential for N. aromaticivorans to also become a source of CoQ₁₀ when grown in aromatic-containing solutions derived from plant biomass.

In this work, we test several predicted reactions in the N. aromaticivorans carotenoid biosynthetic pathway (FIG. 1) by generating defined mutants that accumulate β-carotene, lycopene, or zeaxanthin. Further, we engineer a strain that heterologously expresses a CrtW protein and accumulates the carotenoid astaxanthin. We show that these carotenoids can be produced from vanillate, an aromatic compound commonly present in deconstructed lignocellulosic plant biomass, and from an alkaline pretreatment liquor (APL) made from Sorghum. We also engineer a set of strains that produce either zeaxanthin, β-carotene, or astaxanthin concurrently with PDC when fed Sorghum APL, showing that N. aromaticivorans can be engineered to simultaneously produce extracellular and intracellular products from this renewable carbon source. We discuss how co-production of membrane-bound carotenoids and excreted dicarboxylic acids like PDC could improve the economics of valorizing biomass in a lignocellulosic biorefinery.

### Results

Nostoxanthin is the Main Carotenoid Produced by N. aromaticivorans DSM 12444

The N. aromaticivorans DSM 12444 genome predicts that this bacterium contains genes that encode previously uncharacterized proteins with 52 to 74% amino acid sequence identity to known enzymes that lead to nostoxanthin production (Table 1).

TABLE 1
Amino acid sequence identity of predicted N. aromaticivorans
DSM 12444 carotenoid pathway proteins with the most closely
related characterized protein identified by PaperBLAST (51).

| | | | Most | | |
|---|---|---|---|---|---|
| | | | similar | Organism | |
| | Locus tag, | | protein | encoding | |
| Protein | accession | % | accession | most similar | Reference for |
| annotation | number | identity | number | protein | characterization |
| CrtI | Saro_1816, | 74% | ADO33738.1 | Sphingomonas | (23) |
| | ABD26256.1 | | | elodea ATCC | |
| | | | | 31461 | |
| CrtY | Saro_1817, | 52% | AEP37353.1 | S. elodea | (23) |
| | ABD26257.1 | | | ATCC 31461 | |
| CrtZ | Saro_1168, | 58% | AIT05942.1 | Sphingomonas | (25) |
| | ABD25613.1 | | | taxi ATCC | |
| | | | | 55669 | |
| CrtG | Saro_0236, | 72% | AEP37351.1 | S. elodea | (23) |
| | WP_011443898.1 | | | ATCC 31461 | |

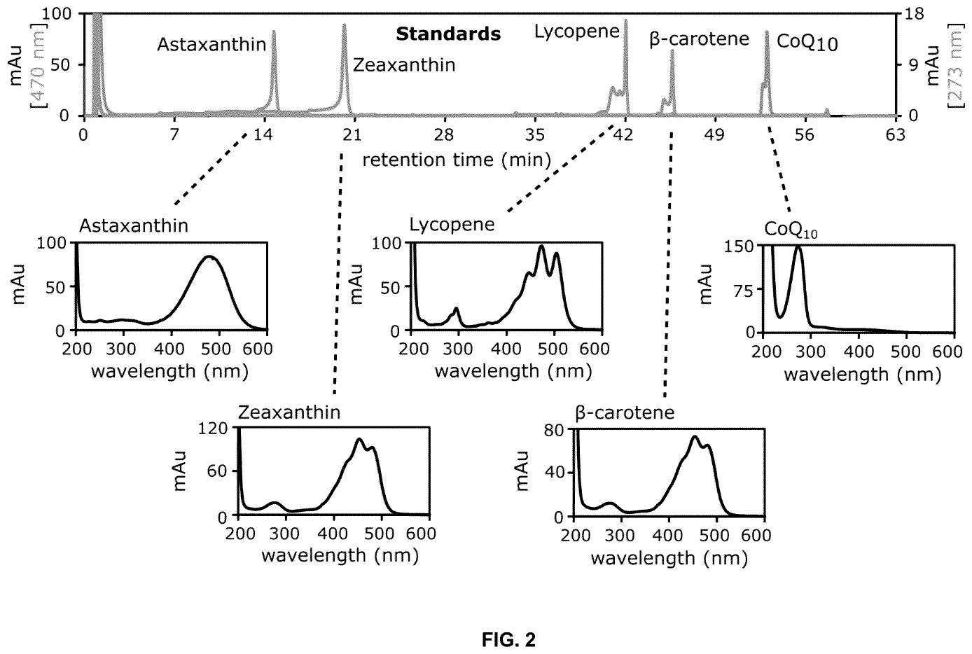

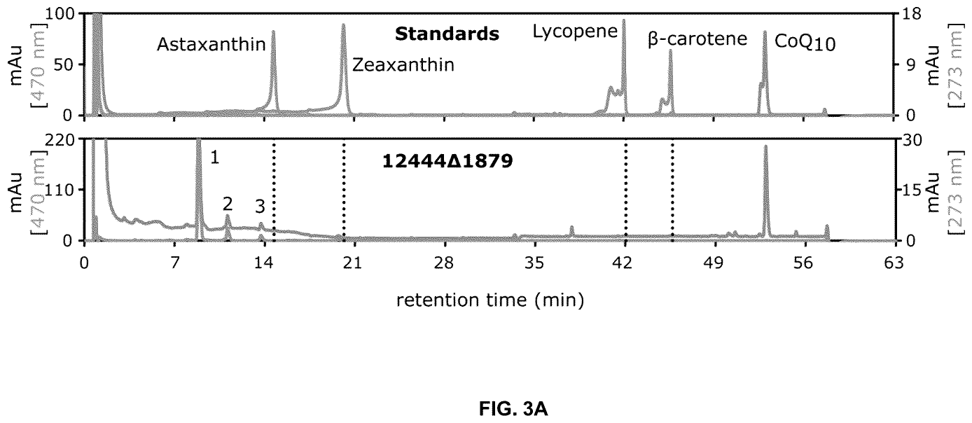

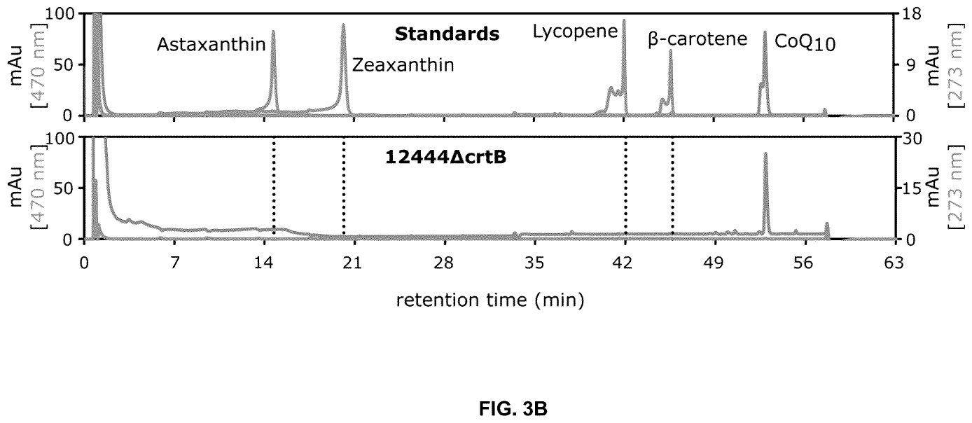

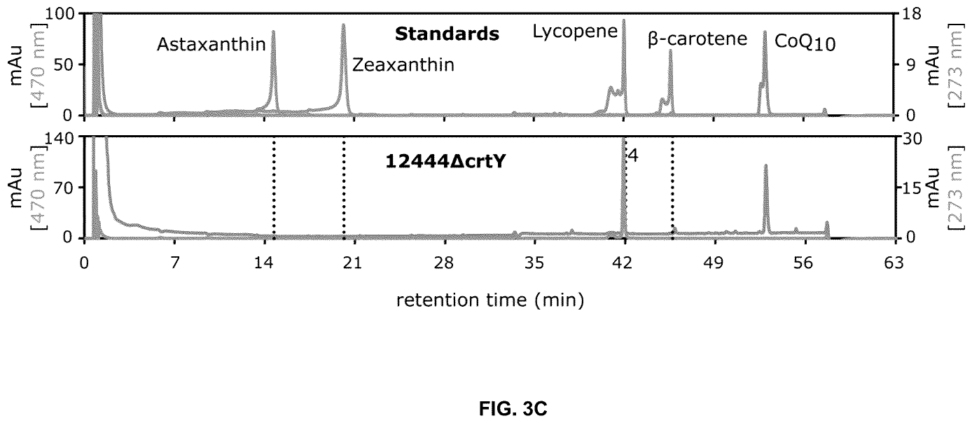

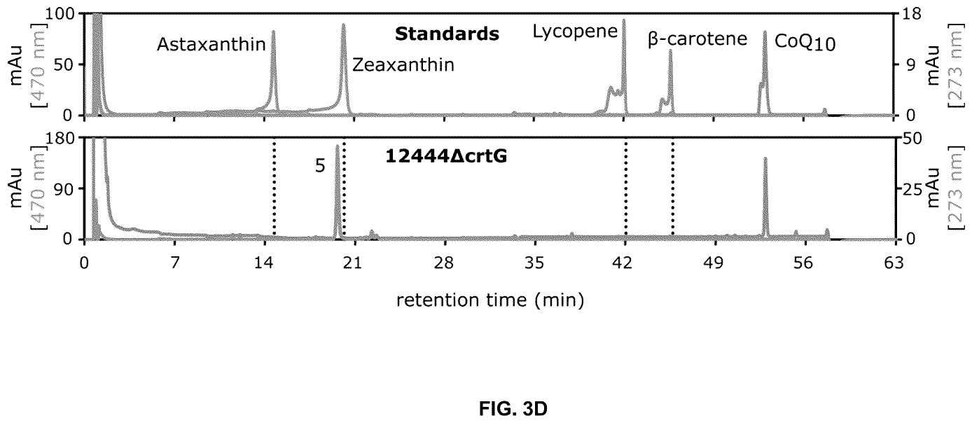

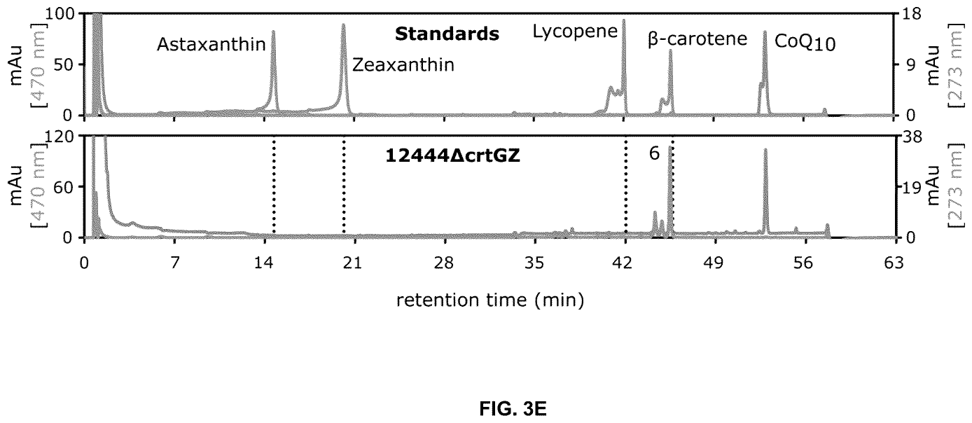

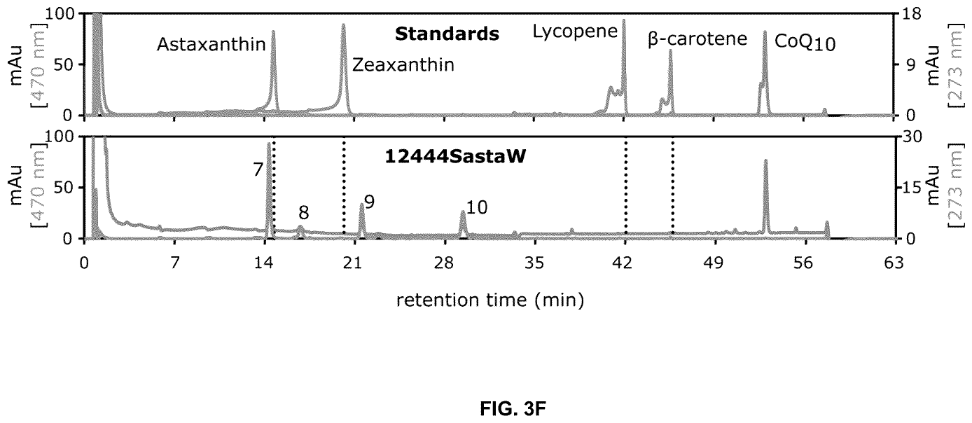

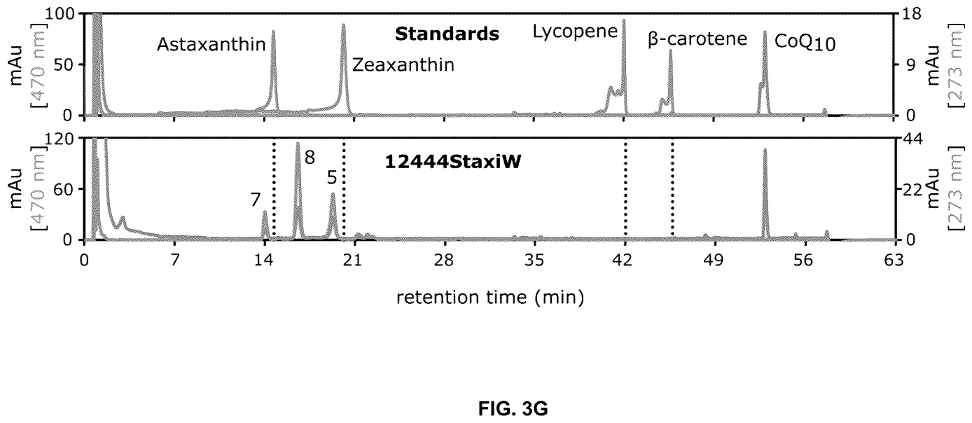

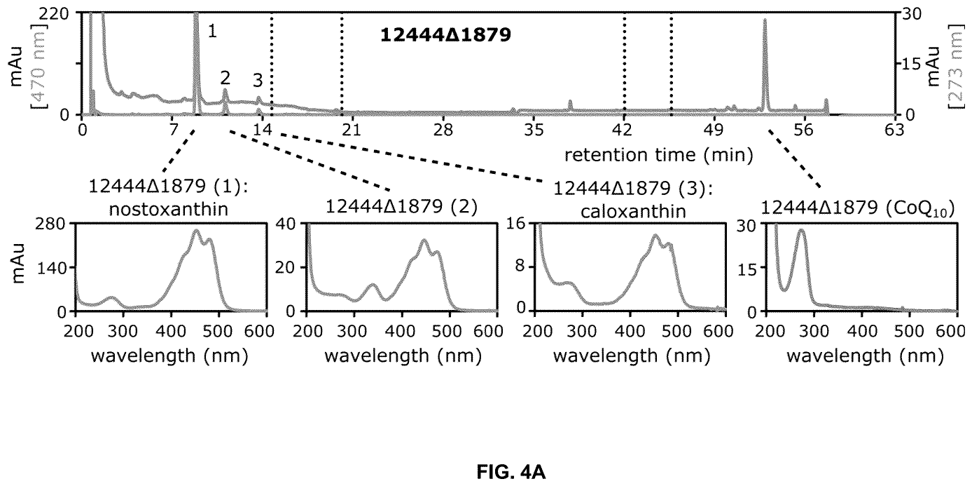

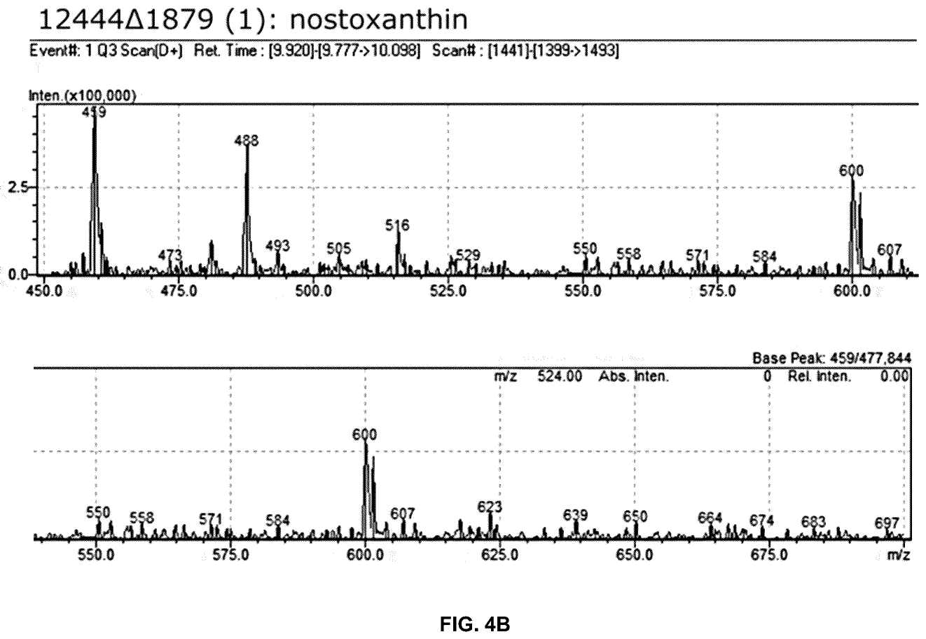

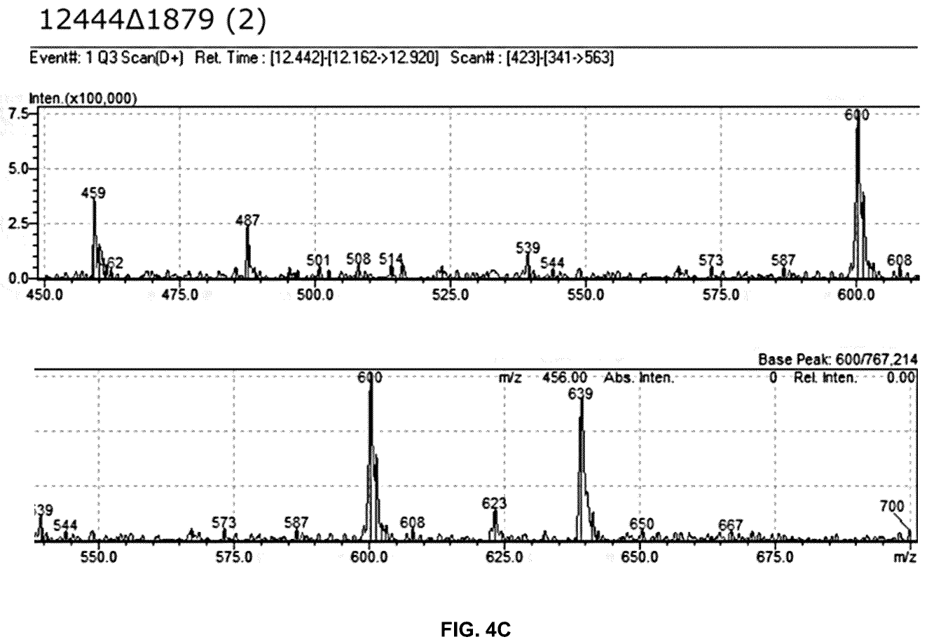

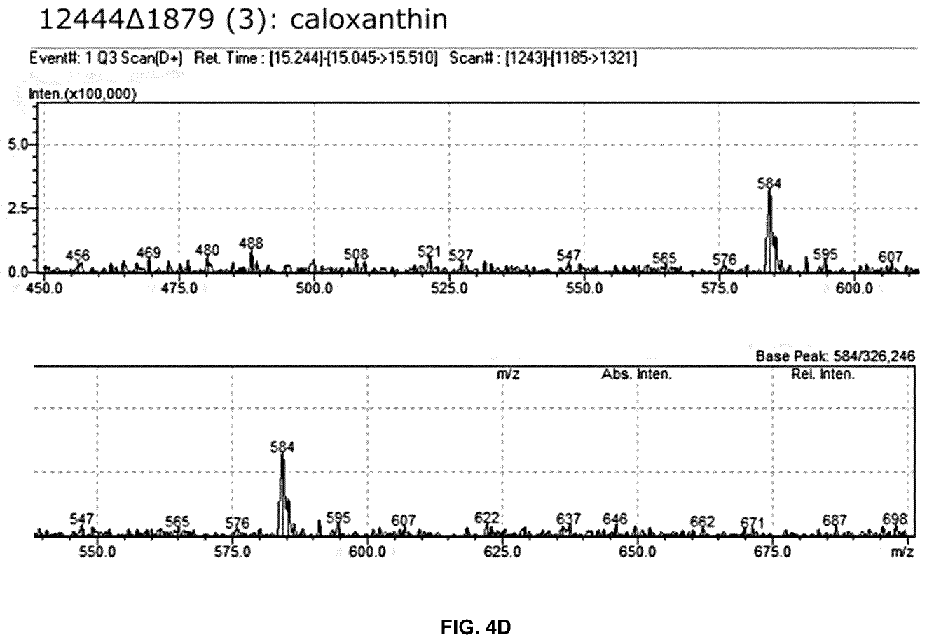

To test the prediction that N. aromaticivorans uses these previously uncharacterized gene products to produce carotenoids, we grew cells in the presence of vanillate and analyzed acetone:methanol (lipophilic) extracts of the cells by LC-MS. Our analysis of these extracts from the parent N. aromaticivorans strain (12444Δ1879) was consistent with nostoxanthin being a major carotenoid (compound 1 in FIG. 3A): compound 1 had absorbance maxima at 453 and 480 nm (FIG. 4A) and an m/z peak of 600 (FIG. 4B), both characteristic of nostoxanthin (21, 22). Two other compounds in these extracts had slightly longer retention times than compound 1: compound 2 had absorbance maxima at 339, 446, and 474 nm (FIG. 4A), and m/z peaks of 600 and 639 (FIG. 4C), and compound 3 had absorbance maxima at 453 and 481 nm (FIG. 4A), and an m/z peak of 584 (FIG. 4D). Though we were not able to identify compound 2, the properties of compound 3 are consistent with caloxanthin (22), a predicted precursor to nostoxanthin (FIG. 1) known to be present in some sphingomonads that produce nostoxanthin (22, 23). However, commercial standards of pure nostoxanthin or caloxanthin were not available to estimate the compounds' abundances or provide further proof of their chemical identity. Another compound detected in the N. aromaticivorans lipophilic extracts had the same retention time and absorbance spectrum as a commercial CoQ₁₀ standard (FIG. 3A and FIGS. 2 and 4A, consistent with previous work showing that CoQ₁₀ is the major quinone in this bacterium (15).

Lipophilic Compounds Produced by N. aromaticivorans Mutants Containing Deletions of Genes in the Predicted Carotenoid Biosynthetic Pathway

To further test whether N. aromaticivorans uses the predicted carotenoid biosynthesis pathway (FIG. 1), we generated a set of mutants with in-frame deletions of genes predicted to encode proteins involved in the pathway. We grew these strains with vanillate as sole carbon source and analyzed the compounds present in lipophilic extracts from these mutants, using commercial standards when available to aid in identification and quantification of the lipophilic compounds.

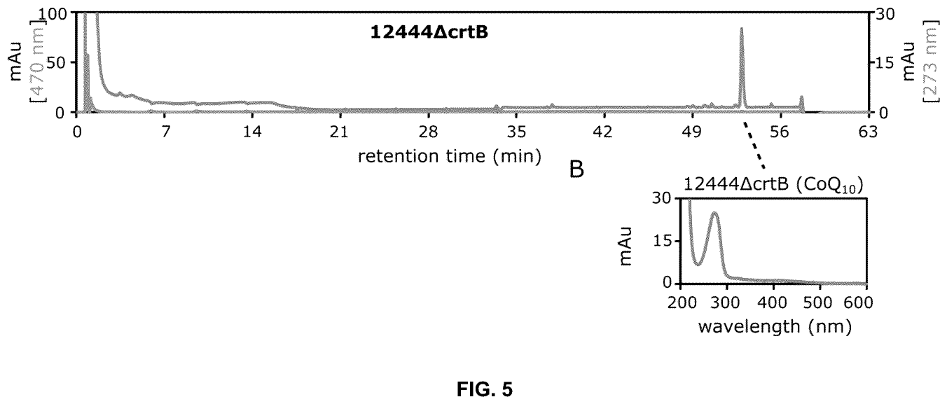

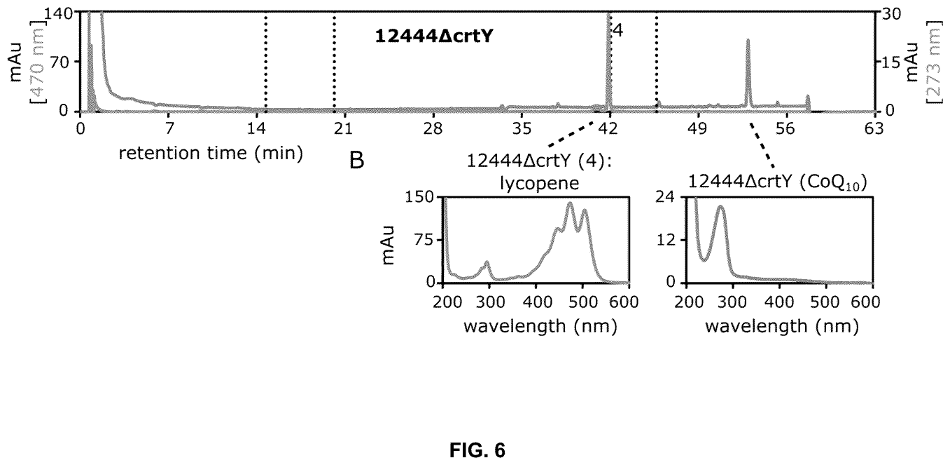

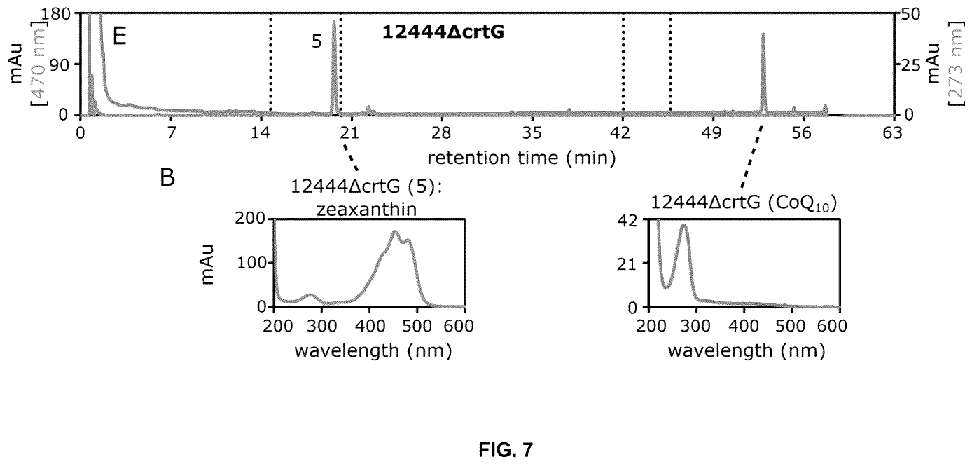

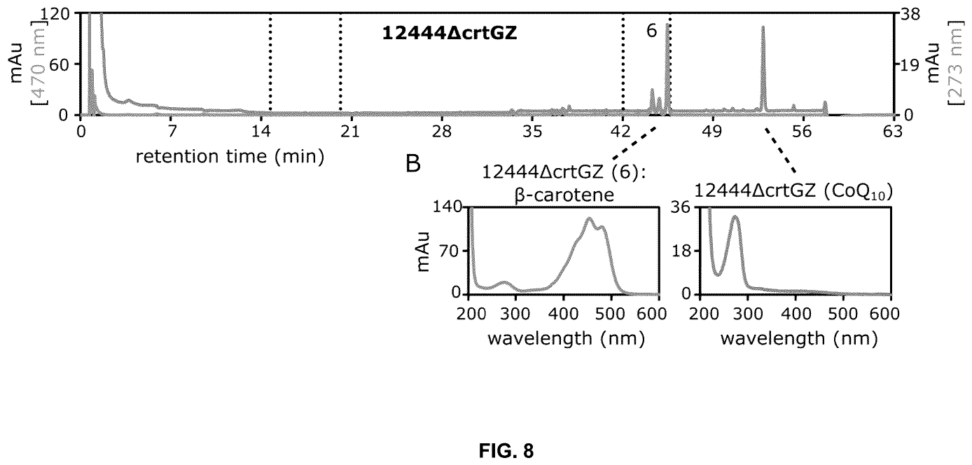

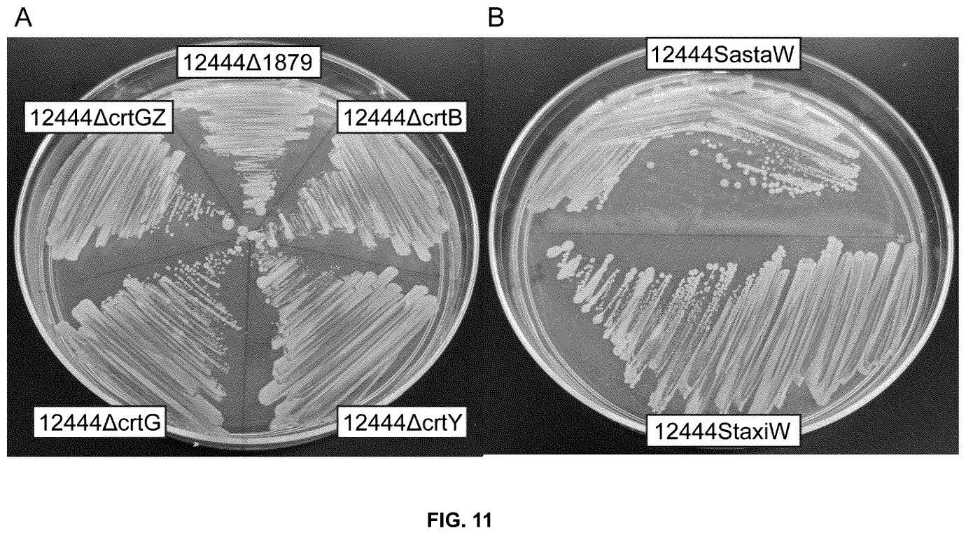

Deletion of Saro_1814 (encoding a putative CrtB homologue) resulted in a strain (12444ΔcrtB) that formed non-pigmented colonies on solid media (FIG. 11 (A, left panel)), as expected given the predicted role of this gene product in phytoene synthesis (FIG. 1). We also found that strain 12444ΔcrtB only contained CoQ₁₀ as a major lipophilic compound (FIG. 3B and FIG. 5). Deletion of Saro_1817 (encoding a putative CrtY homologue) resulted in a strain (12444ΔcrtY) that formed light pink colonies (FIG. 11 (A, left panel)), and contained the predicted pathway intermediate lycopene (compound 4) as well as CoQ₁₀ in its lipophilic extract (FIG. 3C and FIG. 6). Deletion of Saro_0236 (encoding a putative CrtG homologue) resulted in a strain (12444ΔcrtG) that formed yellow colonies (FIG. 11 (A, left panel)) and contained the predicted pathway intermediate zeaxanthin (compound 5) and CoQ₁₀ as the major components of its lipophilic extract (FIG. 3D and FIG. 7). Finally, to test if the pathway intermediate β-carotene could be accumulated, we constructed a strain (12444ΔcrtGZ) in which both Saro_0236 and Saro_1168 (encoding a putative CrtZ homologue) were deleted, as deletion of Saro_1168 alone would be expected to also produce 2,2′-dihydroxy-β-carotene (FIG. 1). Strain 12444ΔcrtGZ formed yellow colonies (FIG. 11 (A, left panel)), and the main components of its lipophilic extract were β-carotene (compound 6) and CoQ₁₀ (FIG. 3E and FIG. 8).

In sum, the compounds present in the lipophilic extracts of each of these mutants were consistent with predictions from the annotated carotenoid biosynthetic pathway in the N. aromaticivorans genome (FIG. 1). These experiments illustrate that one or more gene deletions can result in N. aromaticivorans strains that produce industrially valuable carotenoids when grown in the presence of vanillate. Furthermore, the lipophilic extracts from all strains tested also contain the electron carrier CoQ₁₀.

Production of Astaxanthin by an Engineered N. aromaticivorans Strain

Astaxanthin is a valuable carotenoid that is not predicted to be produced by N. aromaticivorans, since the genome of this organism lacks a crtW gene. While several bacteria, including some other members of the Sphingomonadales order (Sphingomonas astaxanthinifaciens (24) and Sphingomonas taxi ATCC 55669 (25)), naturally produce astaxanthin (26), none of these are known to metabolize aromatic compounds present in pretreated lignocellulosic biomass. To test if we could engineer N. aromaticivorans to produce astaxanthin, we placed a recombinant crtW gene from S. astaxanthinfaciens or S. taxi separately into the crtG locus of 12444ΔcrtG to generate strains 12444SastaW and 12444StaxiW, respectively. The difference in colony colors of the 12444ΔcrtG, 12444SastaW, and 12444StaxiW strains (FIG. 11) suggested that the insertion of each of these crtW genes into the N. aromaticivorans genome resulted in altered carotenoid profiles.

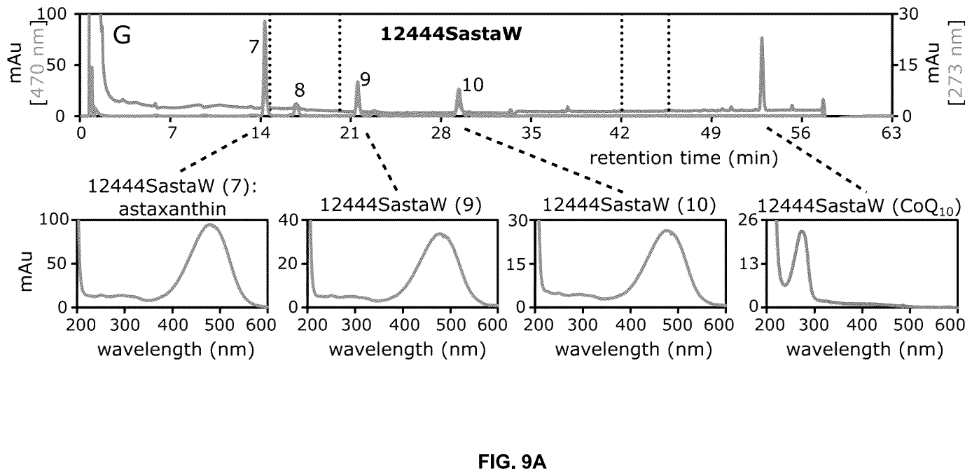

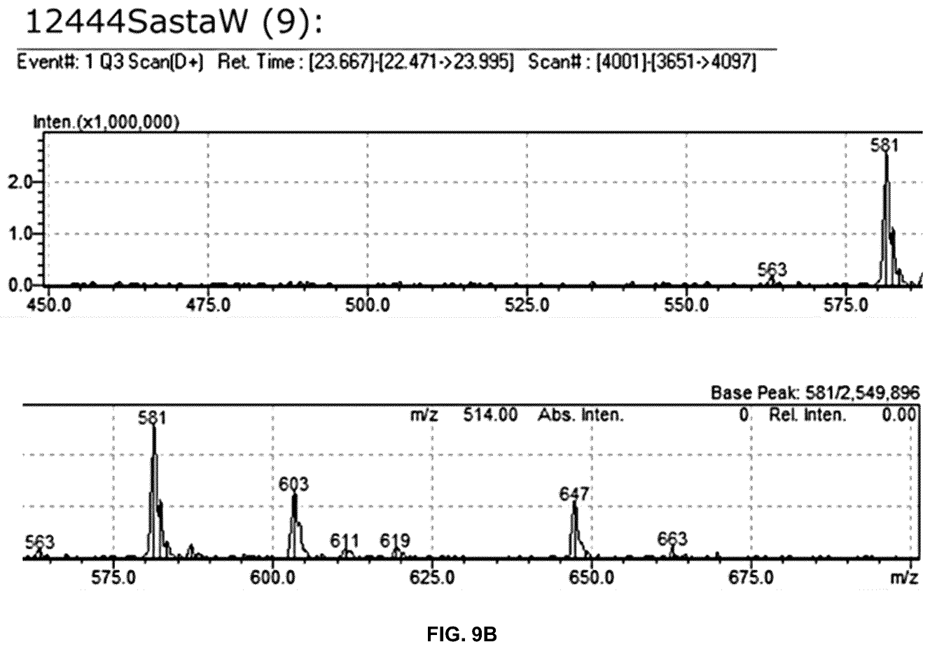

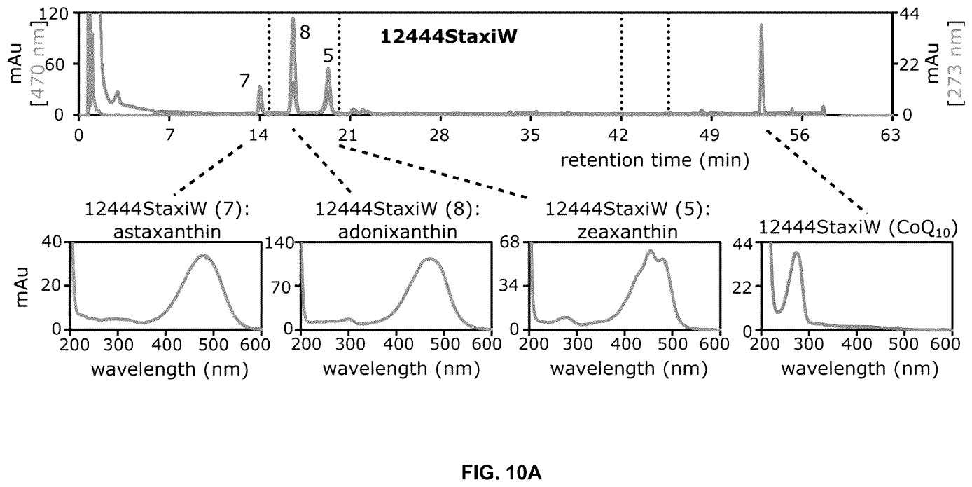

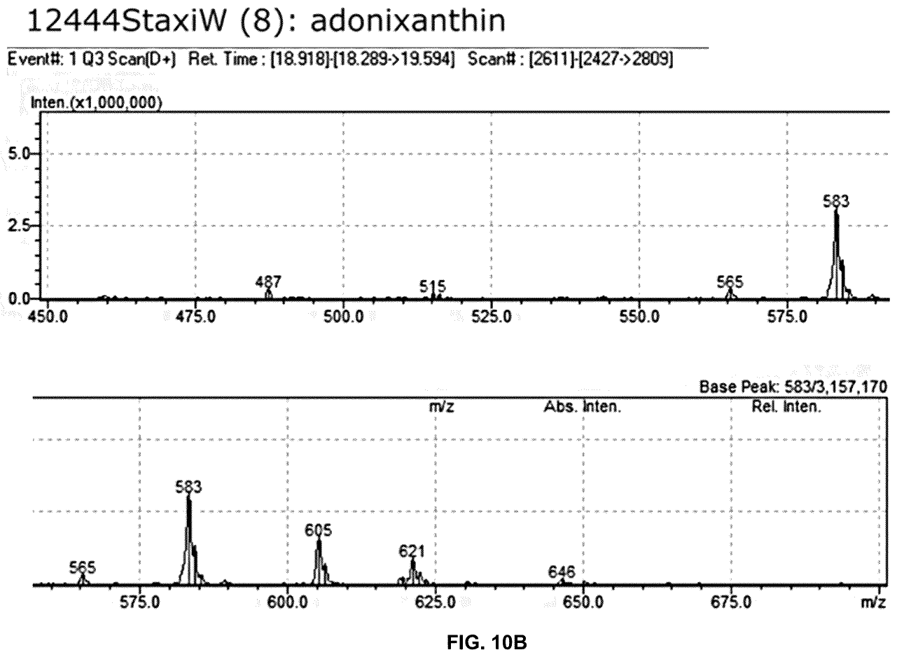

To test this hypothesis, we analyzed the lipophilic extracts from vanillate-grown 12444SastaW and 12444StaxiW cells. This analysis showed that 12444SastaW produced astaxanthin (FIG. 3F; compound 7), as well as small amounts of three other putative carotenoids (compounds 8, 9, 10) and CoQ₁₀ (FIG. 3F and FIG. FIGS. 9A and 9B). The lipophilic extract of 12444StaxiW also contained astaxanthin (compound 7) and CoQ₁₀ (FIG. 3G and FIG. 10A), although its predominant carotenoid (compound 8, which is also a minor component of the 12444SastaW extract) appears to be adonixanthin (FIG. 10A, based on its measured mass (m/z peak=583; FIG. 10B). The 12444StaxiW lipophilic extract also contained two additional compounds, one of them identified as zeaxanthin (compound 5; FIG. 3G and FIG. 10A). From this, we conclude that introducing the CrtW protein from S. astaxanthinfaciens into N. aromaticivorans generates a strain that is more effective at accumulating astaxanthin than a strain using the CrtW protein from S. taxi. Therefore, in subsequent experiments, we used cells containing the S. astaxanthinfaciens crtW gene (strain 12444SastaW) as a chassis for an astaxanthin-producing strain of N. aromaticivorans.

Impact of O₂ Tension on Levels of Carotenoids and CoQ₁₀ in N. aromaticivorans

The dissolved O₂ concentration of a culture can affect carotenoid levels in various organisms in different ways (27). For example, lower O₂ tensions have been shown to increase CoQ₁₀ production in some bacteria (20), while other microbes increase carotenoid production at high O₂ tensions presumably since carotenoids can provide protection against reactive oxygen species (27). Therefore, we tested whether bubbling N. aromaticivorans cultures with gas containing 5%, 10%, or 21% O₂ would lead to significant changes in carotenoids and CoQ₁₀ levels when using vanillate as a carbon source.

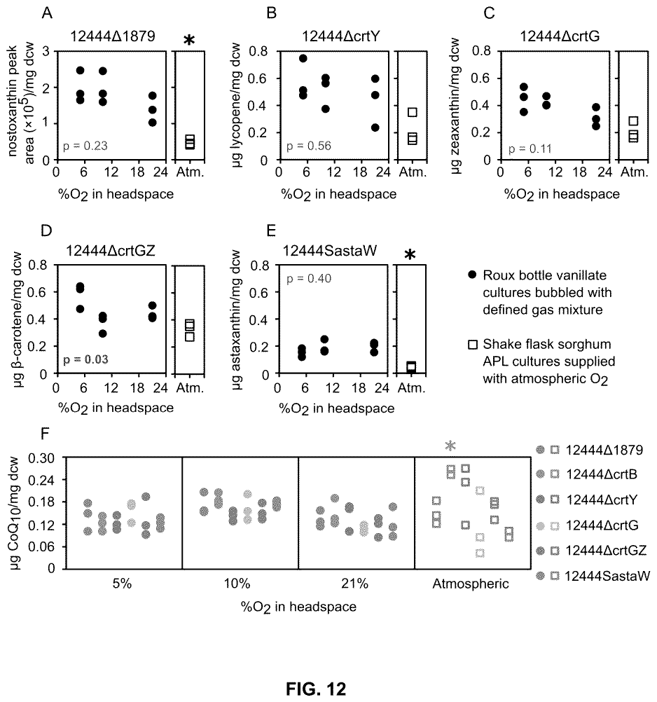

Carotenoid levels of N. aromaticivorans strains grown at different O₂ tensions are shown in FIG. 12 (A-E). Nostoxanthin levels in 12444Δ1879, which are reported as HPLC peak area due to lack of a standard for quantification, showed only modest changes with oxygen (ranging from 1.0 to 2.5×10⁵ area units per mg dry cell weight (dcw)). Under the same growth conditions, lycopene levels in 12444ΔcrtY ranged from 0.24 to 0.75 μg/mg dcw, zeaxanthin in 12444ΔcrtG ranged from 0.25 to 0.54 μg/mg dcw, β-carotene in 12444ΔcrtGZ ranged from 0.29 to 0.64 μg/mg dcw, and astaxanthin in 12444SastaW ranged from 0.12 to 0.25 μg/mg dew. Single-factor ANOVA tests (alpha=0.05) of carotenoid levels suggested that only strain 12444ΔcrtGZ contained significantly different carotenoid levels at the O₂ concentrations tested (FIG. 12 (A-E)). CoQ₁₀ levels for each strain at different O₂ tensions (FIG. 12 (F)) ranged from 0.08 to 0.2 μg/mg dcw. ANOVA tests also showed that, for each of the strains, there was no significant difference in CoQ₁₀ production at the three O₂ concentrations tested (FIG. 12 (F)). Furthermore, ANOVA tests showed there was no significant difference in CoQ₁₀ production between the different strains at any of the individual O₂ concentrations. Therefore, we conclude that O₂ availability does not have a consistent significant impact on N. aromaticivorans carotenoid or CoQ₁₀ levels over the concentration range tested.

Production of Carotenoids and CoQ₁₀ by N. aromaticivorans from Alkaline Pretreated Sorghum Biomass

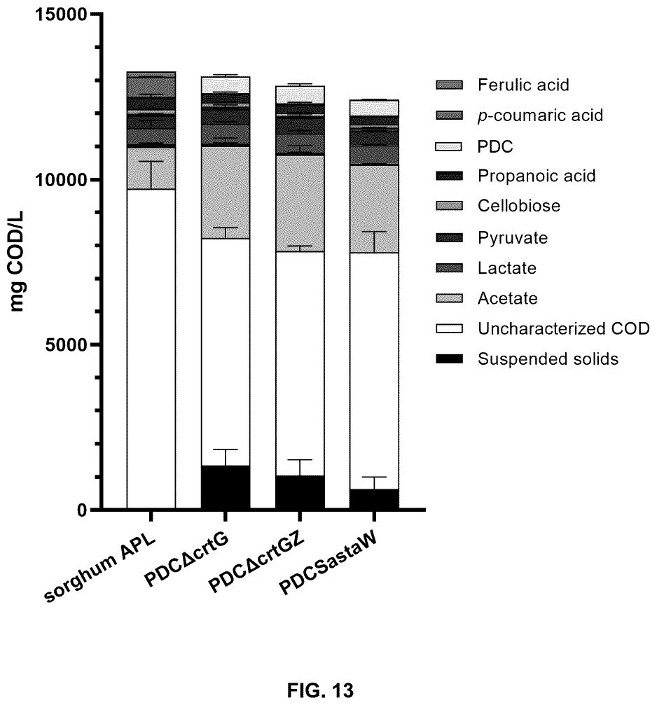

The above results showed that wild type and engineered N. aromaticivorans strains accumulate carotenoids and CoQ₁₀ when grown on vanillate, an aromatic compound predicted to be found in deconstructed lignocellulosic biomass. However, we also wanted to test whether carotenoids and CoQ₁₀ could be generated when cells were grown on the mixture of aromatics directly obtained from plant biomass. We therefore grew several strains in a Sorghum alkaline pretreatment liquor (APL) (28) which contains a mixture of aromatic monomers and other organics (FIG. 13, Table 2). With a few exceptions, the N. aromaticivorans strains grown in Sorghum APL (in shake flasks with atmospheric O₂ conditions) produced amounts of carotenoids and CoQ₁₀ (normalized by dry cell weight; open squares in FIG. 12) that were within the production ranges of the vanillate-grown cultures grown at different dissolved O₂ tensions (filled circles in FIG. 12). One exception was strain 12444ΔcrtB, which lacks detectable carotenoids in lipophilic extracts; this strain produced significantly more CoQ₁₀ when grown in APL compared to when grown on vanillate (FIG. 12 (F)). In addition, strains 12444Δ1879 and 12444SastaW produced significantly less nostoxanthin and astaxanthin, respectively, when grown in APL compared to when grown on vanillate. Overall, these results show that it is possible to use N. aromaticivorans for producing carotenoids from a mixture of aromatic and other organic compounds derived from pretreated lignocellulosic biomass.

TABLE 2
Concentration of aromatic monomers and other organics in sorghum APL
used in this study. Values represent mean ± S.D.. Trace levels
of indicated aromatic compounds were detected, but concentrations <<
0.1 mM prevented quantification. All aromatics in Table 5 were investigated.
Other organics were measured as described herein. ND, not detected.

| | Concentration (mM) | | | |
|---|---|---|---|---|
| | Sorghum APL | PDCΔcrtG | PDCΔcrtGZ | PDCSastaW |
| Compound | (n = 2) | (n = 3) | (n = 3) | (n = 3) |
| Aromatic monomers | | | | |
| p-coumaric acid | 2.01 ± 0.03 | ND | ND | ND |
| Ferulic acid | 0.513 ± 0.001 | ND | ND | ND |
| PDC | ND | 3.2 ± 0.3 | 3.3 ± 0.4 | 2.9 ± 0.1 |
| Acetosyringone | Trace | ND | ND | ND |
| p-hydroxybenzaldehyde | Trace | ND | ND | ND |
| p-hydroxybenzoic acid | Trace | ND | ND | ND |
| Protocatechuic acid | Trace | ND | ND | ND |
| Vanillin | Trace | ND | ND | ND |
| Syringic acid | ND | Trace | ND | Trace |
| Vanillic acid | ND | Trace | ND | Trace |
| Other organics | | | | |
| Acetate | 19.8 ± 0.9 | 43 ± 4 | 45 ± 4 | 41.5 ± 0.3 |
| Lactate | 5 ± 2 | 6.3 ± 0.7 | 6.2 ± 0.9 | 5.8 ± 0.2 |
| Pyruvate | 4.8 ± 0.3 | 5.7 ± 0.3 | 6.1 ± 0.5 | 5.4 ± 0.2 |
| Propanoic acid | 3.2 ± 0.7 | 2.5 ± 0.3 | 2.6 ± 0.2 | 2.44 ± 0.02 |
| Formate | 2.8 ± 0.7 | 2.0 ± 0.2 | 0.98 ± 0.08 | 1.5 ± 0.2 |
| Cellobiose | 0.36 ± 0.03 | 0.32 ± 0.07 | 0.247 ± 0.005 | 0.260 ± 0.006 |
| Xylitol | 0.25 ± 0.06 | 0.4 ± 0.3 | 0.2 ± 0.4 | 0.54 ± 0.06 |
| Glucose | 0.1 ± 0.1 | 0.1 ± 0.1 | 0.1 ± 0.1 | ND |
| Ethanol | 0.03 ± 0.02 | 0.2 ± 0.1 | 0.09 ± 0.08 | 0.08 ± 0.09 |
| Glycerol | 0.2 ± 0.2 | ND | ND | ND |
| Succinate | ND | ND | ND | ND |
| Xylose | ND | ND | ND | ND |

Concurrent Production of Carotenoids, CoQ₁₀, and PDC from Alkaline Pretreated Sorghum Biomass

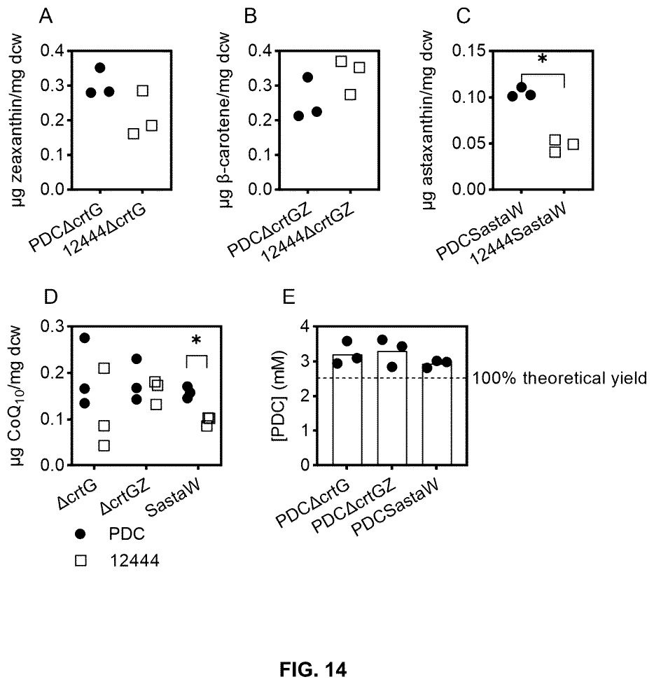

Previous work has shown that engineered N. aromaticivorans strains containing defined mutations in aromatic metabolism can convert the three major classes of biomass aromatics (syringyl, guaiacyl, and p-hydroxyphenyl) into PDC and secrete it into the medium (2, 7). We sought to test whether individual N. aromaticivorans strains could produce both extracellular PDC and intracellular lipophilic compounds (carotenoids and CoQ₁₀) as valuable products from biomass-derived media. To do this, we generated a set of strains that contained both the mutations needed to accumulate extracellular PDC and those needed to accumulate the carotenoids zeaxanthin, β-carotene, or astaxanthin. We found that these engineered strains produce extracellular PDC, as well as the expected carotenoid and CoQ₁₀, when grown in Sorghum APL (FIG. 14). The levels of individual carotenoid species and CoQ₁₀ (normalized by dry cell weight) in the lipophilic cell extracts are comparable to or greater than the amounts produced by the respective non-PDC-producing strains grown in Sorghum APL (FIG. 14 (A-D)). In addition, the levels of extracellular PDC are equal to or greater than 100% theoretical yield, based on the measured concentrations of aromatic compounds in APL (FIG. 14 (E), Table 2). This observation is consistent with the stoichiometric conversion of aromatic monomers to PDC reported previously for the PDC-producing strain that was used in these studies (7).

### DISCUSSION

This work sought to expand the types of valuable chemicals that could be produced from pretreated lignocellulosic biomass. We confirmed that N. aromaticivorans naturally produces the industrially important isoprenoid CoQ₁₀, and we leveraged its native ability to synthesize carotenoids along with the utility of heterologous expression to engineer mutant strains that accumulate different valuable carotenoids from either a pure aromatic (vanillate) or from Sorghum APL, a feedstock derived from lignocellulosic biomass. We also used this new information to engineer a set of N. aromaticivorans strains that can concurrently produce CoQ₁₀, a valuable carotenoid, and PDC from Sorghum APL.

N. aromaticivorans as a Production Platform for Carotenoids and CoQ₁₀

Although other microbes can be used as sources of carotenoids and CoQ₁₀, our work is important for several reasons. First, we confirmed predictions from N. aromaticivorans genomic and physiological studies that this bacterium contains metabolic pathways that can produce the carotenoid nostoxanthin (13) as well as CoQ₁₀ (15). Our work also shows that minimal genomic modifications of N. aromaticivorans can lead to strains that accumulate valuable carotenoids such as β-carotene, lycopene, astaxanthin, or zeaxanthin. In addition, we demonstrate that N. aromaticivorans cells lacking crtB (Saro_1814) could simplify industrial production of CoQ₁₀, another important commodity chemical used in the pharmaceutical and cosmetics industries, because the crtB mutation prevents the accumulation of other acetone:methanol soluble materials. We also found that changes in O₂ tension did not have a significant impact on N. aromaticivorans carotenoid or CoQ₁₀ levels, unlike in other microbes where O₂ tensions or the resulting oxidative stress can have a significant impact on accumulation of these products (27). Effects of O₂ tension on carotenoid production can be variable; in some cases lower O₂ tensions lead to higher production due to increased membrane synthesis (29) while in other cases higher O₂ tensions lead to higher production (30) presumably due to the ability of these compounds to quench reactive oxygen species (27). While our data suggests that N. aromaticivorans might not regulate carotenoid synthesis in response to changes in O₂ tension, additional studies are needed to test this hypothesis. The ability of N. aromaticivorans to synthesize comparable levels of carotenoids and CoQ₁₀ at different 02 concentrations could be advantageous in industrial settings, due to the capital and operational costs associated with aeration of large bioreactors.

Microbial Production of Valuable Commodity Chemicals from Pretreated Lignocellulosic Biomass

As society looks for ways to produce commodity chemicals from abundant renewable resources, pretreated lignocellulosic biomass is an attractive material. N. aromaticivorans has natural converging pathways to catabolize major components found in pretreated lignocellulosic biomass, including the most abundant aromatic monomers found in lignin (syringyl, guaiacyl, and p-hydroxyphenyl aromatics) (2), some aromatic dimers (3, 5), and other organic compounds (31). Because carotenoids and CoQ₁₀ are produced from central metabolites, N. aromaticivorans could thus “funnel” mixtures of compounds found in pretreated lignocellulosic biomass into these commodity chemicals. This is in contrast to existing microbial hosts for producing carotenoids or CoQ₁₀ that typically use food-grade sugars as carbon sources (32). To date, relatively few of the microbes being considered for industrially producing carotenoids and CoQ₁₀ also have the native ability to metabolize any aromatic compounds (33-37). Thus, N. aromaticivorans could be an important microbial catalyst for industrial production of carotenoids and CoQ₁₀ from renewable lignocellulosic carbon sources.

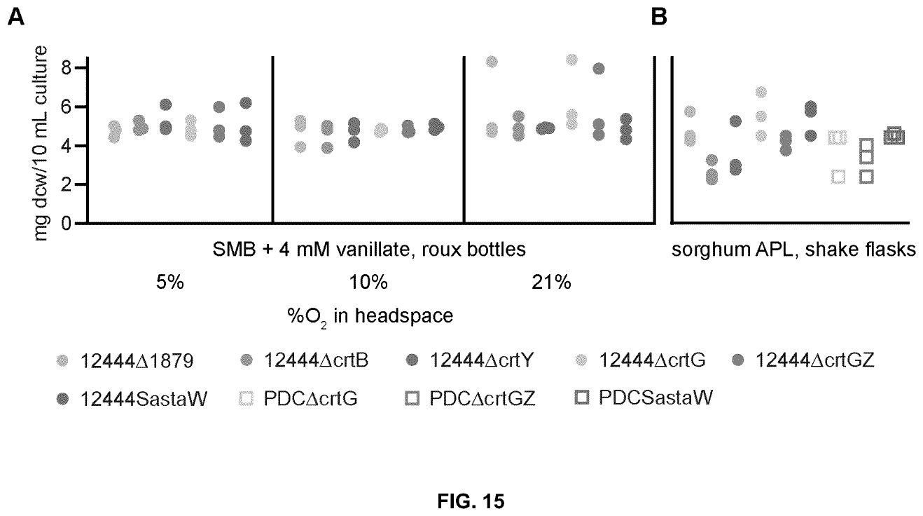

The method used to generate the Sorghum APL feedstock is well known to solubilize easily cleavable aromatics from plant cell walls, without the breakdown of the carbohydrate and lignin polymers in the biomass (28). We showed that N. aromaticivorans can grow in the presence of Sorghum APL alone, unlike previous studies which have supplemented lignocellulosic APL with minerals (38-43) and/or nitrogen/carbon sources (38, 40, 44, 45). We also found that N. aromaticivorans produces nearly the same amounts (normalized to dry cell weight) of carotenoids and CoQ₁₀ when grown on Sorghum APL as when grown in a defined medium containing vanillate (FIG. 12), in addition to accumulating comparable amounts of cellular material (FIG. 15). These results suggest that N. aromaticivorans could produce these valuable isoprenoids from lignocellulosic substrates without the need to add other nutrients.

Simultaneous Production of Carotenoids, CoQ₁₀ and PDC from Pretreated Lignocellulosic Biomass

Our work illustrates the potential for N. aromaticivorans to produce carotenoids and CoQ₁₀ as intracellular lipophilic products. We also generated strains that concurrently produce these lipophilic products along with the soluble extracellular product PDC, which has several potential industrial uses (46). In strains that accumulate both intracellular (carotenoids and CoQ₁₀) and extracellular (PDC) products, the cellular and aqueous fractions can be separated and each used as a source of valuable products to increase the economic value derived from pretreated lignocellulosic biomass. Notably, the strains we engineered for simultaneous PDC and carotenoid production accumulate at least as much carotenoid as the non-PDC-producing strains, showing that synthesis of two products does not have a significant negative impact on the overall output. The greater than 100% theoretical yield of PDC observed for these strains (FIG. 14 (E)) likely reflects conversion of aromatic compounds present in the Sorghum APL that were not detected by our HPLC-MS/MS analysis, in addition to complete conversion of detected aromatic monomers (Table 2).

In previous studies with strains that produce PDC from aromatics, a second carbon source (glucose) was added to cells, since the mutations that result in accumulation of PDC block the use of aromatics to support growth (2, 7). The growth of PDC-producing strains in Sorghum APL reported here predicts that N. aromaticivorans will not need to be supplemented with other nutrients to produce extracellular and intracellular compounds from this and possibly other types of feedstocks derived from plant biomass.

In sum, this work adds to a growing body of evidence that N. aromaticivorans is a promising microbe for converting lignocellulosic biomass into valuable compounds because it is amenable to genomic modifications and can metabolize abundant aromatic components of this biomass. This work establishes N. aromaticivorans as a promising host for producing valuable carotenoids and CoQ₁₀ from both pretreated lignocellulosic biomass and from purified aromatics. In addition, N. aromaticivorans has the ability to produce these intracellular lipophilic compounds concurrently with PDC, which could help to improve the economics of converting plant biomass into industrial commodities. Future work will focus on improving yields of these and other products under industrially relevant conditions.

### Materials and Methods

Novosphingobium aromaticivorans Strains

Details on all strains in this study can be found in Table 3. N. aromaticivorans 12444Δ1879 is a derivative of wild-type strain DSM 12444 (also called F199 (31, 47)), in which a putative sacB gene (Saro_1879 or SARO_RS09410) was deleted to create a strain amenable to genomic modifications using a sacB-containing plasmid (3, 48). We used 12444Δ1879 as the parent strain to generate strains 12444ΔcrtB (lacking crtB; Saro_1814 or SARO_RS09080), 12444ΔcrtY (lacking crtY; Saro_1817 or SARO_RS09095), 12444ΔcrtG (lacking crtG; Saro_0236 or SARO_RS01180), 12444ΔcrtGZ (lacking crtG and crtZ; Saro_0236 and Saro_1168 or SARO_RS01180 and SARO_RS05825), 12444StaxiW (replacing Saro_0236 with the gene for the CrtW protein from S. taxi ATCC 55669 (NCBI Accession WP_038660513.1)), and 12444SastaW (replacing Saro_0236 with the gene for the CrtW protein from S. astaxanthinfaciens (NCBI accession WP_211248127.1)).

TABLE 3
All strains and plasmids used in this study.

| Name | Genotype | Description | Reference |
|---|---|---|---|
| E. coli strains | | | |
| DH5α | F− Φ80lacZΔM15 | Used for creating | Bethseda |
| | Δ(lacZYA-argF) U169 | and maintaining | Research |
| | recA1 endA1 hsdR17 | plasmids | Laboratories |
| | (rK−, mk+) phoA | | |
| | supE44 λ-thi− gyrA96 | | |
| | relA1 | | |
| S17-1 | recA pro hsdR RP4-2- | Used for mobilizing | (50) |
| | Tc::Mu-Km::Tn7 | plasmids into N. | |
| | | aromaticivorans | |
| | | via conjugation | |
| N. aromaticivorans | | | |
| strains | | | |
| 12444Δ1879 | DSM 12444 | Parent strain; | (3) |
| | ΔSaro_1879 | putative sacB has | |
| | | been deleted to | |
| | | allow genomic | |
| | | modifications using | |
| | | a sacB-containing | |
| | | plasmid | |
| 12444ΔcrtB | DSM 12444 | Parent with deleted | This work |
| | ΔSaro_1879 | crtB | |
| | ΔSaro_1814 | | |
| 12444ΔcrtY | DSM 12444 | Parent with deleted | This work |
| | ΔSaro_1879 | crtY | |
| | ΔSaro_1817 | | |
| 12444ΔcrtG | DSM 12444 | Parent with deleted | This work |
| | ΔSaro_1879 | crtG | |
| | ΔSaro_0236 | | |
| 12444ΔcrtGZ | DSM 12444 | Parent with deleted | This work |
| | ΔSaro_1879 | crtG and crtZ | |
| | ΔSaro_0236 | | |
| | ΔSaro_1168 | | |
| 12444StaxiW | DSM 12444 | Parent with deleted | This work |
| | ΔSaro_1879 | crtG, with the gene | |
| | ΔSaro_0236::crtW | for the CrtW | |
| | from S. taxi ATCC | protein from | |
| | 55669 | Sphingomonas taxi | |
| | | ATCC 55669 | |
| | | (NCBI Accession | |
| | | WP_038660513.1) | |
| | | in the Saro_0236 | |
| | | genomic locus | |
| 12444SastaW | DSM 12444 | Parent with deleted | This work |
| | ΔSaro_1879 | crtG, with the gene | |
| | ΔSaro_0236::crtW | for the CrtW | |
| | from S. | protein from | |
| | astaxanthinifaciens | Sphingomonas | |
| | | astaxanthinifaciens | |
| | | (NCBI accession | |
| | | WP_211248127.1) | |
| | | in the Saro_0236 | |
| | | genomic locus | |
| 12444PDCΔdmtS | DSM 12444 | Parent with deleted | (7) |
| | ΔSaro_1879 | ligI, desCD, dmtS | |
| | ΔSaro_2819 | that accumulates | |
| | ΔSaro_2864-5 | PDC from aromatic | |
| | ΔSaro_1872 | monomers | |
| PDCΔcrtG | DSM 12444 | 12444PDCΔdmtS | This work |
| | ΔSaro_1879 | with deleted crtG | |
| | ΔSaro_2819 | | |
| | ΔSaro_2864-5 | | |
| | ΔSaro_1872 | | |
| | ΔSaro_0236 | | |
| PDCΔcrtGZ | DSM 12444 | 12444PDCΔdmtS | This work |
| | ΔSaro_1879 | with deleted crtG | |
| | ΔSaro_2819 | and crtZ | |
| | ΔSaro_2864-5 | | |
| | ΔSaro_1872 | | |
| | ΔSaro_0236 | | |
| | ΔSaro_1168 | | |
| PDCSastaW | DSM 12444 | 12444PDCΔdmtS | This work |
| | ΔSaro_1879 | with deleted crtG, | |
| | ΔSaro_2819 | with the gene for | |
| | ΔSaro_2864-5 | the CrtW protein | |
| | ΔSaro_1872 | from S. | |
| | ΔSaro_0236::crtW | astaxanthinifaciens | |
| | from S. | in the Saro_0236 | |
| | axtaxanthinifaciens | genomic locus | |
| Plasmids | | | |
| pK18msB-MCS1 | | pK18mobsacB | (3, 48) |
| | | lacking the multiple | |
| | | cloning site, with a | |
| | | new XbaI site | |
| | | introduced | |
| pK18msB/ΔSaro_1814 | | pK18msB-MCS1 | This work |
| | | containing genomic | |
| | | regions that | |
| | | naturally flank | |
| | | Saro_1814 | |
| pK18msB/ΔSaro_1817 | | pK18msB-MCS1 | This work |
| | | containing genomic | |
| | | regions that | |
| | | naturally flank | |
| | | Saro_1817 | |
| pK18msB/ΔSaro_0236 | | pK18msB-MCS1 | This work |
| | | containing genomic | |
| | | regions that | |
| | | naturally flank | |
| | | Saro_0236 | |
| pK18msB/ΔSaro_1168 | | pK18msB-MCS1 | This work |
| | | containing genomic | |
| | | regions that | |
| | | naturally flank | |
| | | Saro_1168 | |
| pK18msB/ΔSaro_0236::StaxiW | | pK18msB/ΔSaro_02 | This work |
| | | 36 with the gene for | |
| | | CrtW from S. taxi | |
| | | ATCC 55669 | |
| | | between the | |
| | | Saro_0236 flanking | |
| | | regions | |
| pK18msB/ΔSaro_0236::SastaW | | pK18msB/ΔSaro_02 | This work |
| | | 36 with the gene for | |
| | | CrtW from S. | |
| | | astaxanthinifaciens | |
| | | between the | |
| | | Saro_0236 flanking | |
| | | regions | |

N. aromaticivorans 12444PDCΔdmtS is a derivative of 12444Δ1879 that was genetically modified to accumulate stoichiometric amounts of PDC from syringyl, guaiacyl, and p-hydroxyphenyl aromatic compounds (7). Strain 12444PDCΔdmtS has Saro_2819 (ligl), Saro_2864-5 (desCD), and Saro_1872 (dmtS) deleted from the genome. We used 12444PDCΔdmtS as the parent strain to generate strains PDCΔcrtG (lacking Saro_0236), PDCΔcrtGZ (lacking both Saro_0236 and Saro_1168), and PDCSastaW (replacing Saro_0236 with the gene for the CrtW protein from S. astaxanthinifaciens).

Genes for CrtW proteins were synthesized as gBlocks (Integrated DNA Technologies, Coralville, IA). Plasmids for cloning were constructed with the NEBluilder HiFi DNA Assembly Master Mix (New England Biolabs; Ipswich, MA). Methods for constructing mutants (including PCR primers used (Table 4)) are contained in Supporting Information.

TABLE 4
Primers used to create cloning vectors for genomic modifications of N. aromaticivorans.

| Gene | | |
|---|---|---|
| deleted/ | Fragment | |
| plasmid | (relative to | |
| linearized | gene) | Primers |
| crtB | Upstream | 5′-cgattcattaatgcagctggcacgacagCAGGACTCTCGATCT |
| (Saro_1814) | | ACCTGCACCATC-3′ (SEQ ID NO: 23) |
| | | 5′-CGATAAAGCCCAGCTTGCTCACAGGTCGTCGGC |
| | | CTTCATTGC-3′ (SEQ ID NO: 24) |
| | Downstream | 5′-GAAGGCCGACGACCTGTGAGCAAGCTGGGCTTT |
| | | ATCGGCAAAGC-3′ (SEQ ID NO: 25) |
| | | 5′-gtttctgcggactggctttctagatgttcCACCATGACGAGGTGG |
| | | ACCAGAATGAAC-3′ (SEQ ID NO: 26) |
| crtY | Upstream | 5′-cgattcattaatgcagctggcacgacagCTTGAAACGGTAGCC |
| (Saro_1817) | | GAAGGTGTAAAGGTCG-3′ (SEQ ID NO: 27) |
| | | 5′-GCAAATGAAAGTGGGTTGGCGATCCGCTTAGGG |
| | | ACATGCGGTTG-3′ (SEQ ID NO: 28) |
| | Downstream | 5′-CATGTCCCTAAGCGGATCGCCAACCCACTTTCA |
| | | TTTGCAGGAACC-3′ (SEQ ID NO: 29) |
| | | 5′-gtttctgcggactggctttctagatgttcGATGGTGCAGGTAGAT |
| | | CGAGAAGTCCTG-3′ (SEQ ID NO: 30) |
| crtG | Upstream | 5′-cgattcattaatgcagctggcacgacagGTCGAACAGTACGTC |
| (Saro_0236) | | ACCTTCATCAACCAG-3′ (SEQ ID NO: 31) |
| | | 5′-CGGTATTGCTCGTGATGCCAACGGCTCCTGCCT |
| | | GAACAG-3′ (SEQ ID NO: 32) |
| | Downstream | 5′-GCAGGAGCCGTTGGCATCACGAGCAATACCGCT |
| | | GCAACTATGG-3′ (SEQ ID NO: 33) |
| | | 5′-gtttctgcggactggctttctagatgttcCTCGTATCCCACAGCGA |
| | | TATCAGGATGC-3′ (SEQ ID NO: 34) |
| crtZ | Upstream | 5′-cgattcattaatgcagctggcacgacagCACTTCCATCGTCTTC |
| (Saro_1168) | | GACTGCTTGAG-3′ (SEQ ID NO: 35) |
| | | 5′-CCTGCTTCAGCACCGCAGCGACACTTTCTTACA |
| | | ATTTGCCCGAAAGTC-3′ (SEQ ID NO: 36) |
| | Downstream | 5′-GGCAAATTGTAAGAAAGTGTCGCTGCGGTGCT |
| | | GAAGCAGGAACTG-3′ (SEQ ID NO: 37) |
| | | 5′-gtttctgcggactggctttctagatgttcCTACTGCCGGATTTTCC |
| | | GGCATGGAAG-3′ (SEQ ID NO: 38) |
| pK18msB- | | 5′-CTGTCGTGCCAGCTGCATTAATG-3′ (SEQ ID |
| MCS1 | | NO: 39) |
| | | 5′-GAACATCTAGAAAGCCAGTCCGCAGAAAC-3′ |
| | | (SEQ ID NO: 40) |
| pK18msB/ | | 5′-CCAACGGCTCCTGCCTGAACAG-3′ (SEQ ID NO: 41) |
| ΔSaro0236 | | 5′-CATCACGAGCAATACCGCTGCAACTATGG-3′ |
| | | (SEQ ID NO: 42) |

Underlined sequences are extensions to the primers that will not bind to the genomic DNA region being amplified. Lowercase bold and bold italicized sequences are complementary to the end regions of linearized pK18msB-MCS1. The upper case BOLD and BOLD ITALICIZED are complementary to the upper case BOLD and BOLD ITALICIZED sequences of the primer used to create the other flanking region for the same gene.

### Bacterial Growth

E. coli strains used for plasmid cloning were grown in lysogeny broth and shaken at ˜200 rpm at 30 or 37° C. For routine manipulation, N. aromaticivorans cultures were grown in GluSis at 30° C. GluSis is a modification of Sistrom's minimal medium (49) in which the succinate has been replaced by 22.6 mM glucose. The minimal medium used for N. aromaticivorans experiments was Standard Mineral Base (SMB) (3) at an initial pH of 7.0. Where needed to select for the presence or absence of plasmids, media were supplemented with 100 μg/mL ampicillin, 50 μg/mL kanamycin, or 10% sucrose (w/v).

### Preparation of Sorghum APL

Sorghum APL (45) was prepared by mixing samples of milled 2014 GLBRC Sorghum (2 g) with a sodium hydroxide solution (1% NaOH in H₂O, 20 mL) in sealed 125 mL Erlenmeyer flasks, before heating for 90 min in an oil bath at 90° C. The flask was then immediately placed in ice for 10 min, after which the biomass and aqueous phases were separated by centrifugation at 4,300×g for 15 min and the supernatant recovered as a source of soluble aromatics. The solid biomass was rinsed three times with ddH₂O (20 mL, 15 mL, and 15 mL), and the washes were recovered through centrifugation. The initial aqueous supernatant and washes were combined and adjusted to pH 7.0 using 1 M HCl. The solution was centrifuged at 20,000×g for 1 h at 4° C. and passed through a 0.2 μm surfactant-free cellulose acetate (SFCA) filter to remove any remaining insoluble material, yielding the alkaline pretreatment liquor (APL) used in further experiments.

Growth of N. aromaticivorans in Minimal Medium with Vanillate

Cultures of each N. aromaticivorans strain were initially grown in a 125 mL conical shake flask containing 10 mL SMB supplemented with 4 mM vanillate. Between 3 and 8 mL of this culture was combined with 480 mL of fresh SMB+4 mM vanillate in a glass roux bottle. Roux bottle cultures were attached to a gas mixer using SideTrak 840 mass flow controllers attached to a FloBox 954 (Sierra Instruments; Monterey, CA) in a 30° C. temperature-controlled room. Gas was piped into the bottoms of the cultures and exhausted from the headspace through outlets in stoppers. The gas contained 5, 10, or 21% O₂, 1% CO₂, and N₂ as the remainder. Cell growth was monitored by periodically removing samples for analysis using a Klett-Summerson photoelectric colorimeter with a red filter. Cultures were grown until they reached late exponential growth or early stationary phase. For dry cell weight (dcw) determination, aliquots (˜80 mL) were centrifuged in pre-weighed tubes (8,000×g for 15 min), supernatants were removed, cell pellets were air-dried in a fume hood, then the tubes were re-weighed (FIG. 15). Aliquots (˜160 mL) were also harvested (centrifuged at 8,000×g for 15 min) for isolation of lipophilic compounds by extraction with acetone:methanol (see below).

Growth of N. aromaticivorans in Sorghum APL

Each N. aromaticivorans strain was initially grown in a 125 mL conical shake flask containing 10 mL SMB supplemented with 10 mM glucose. 1 mL aliquots were centrifuged at ˜7,000×g for 5 min, the supernatant was removed, and the cell pellet was used to inoculate 18 mL of Sorghum APL in a 125 mL conical shake flask. Cultures were shaken at ˜200 rpm at 30° C. until they reached early stationary phase. Aliquots of cultures for extraction into acetone:methanol (10 mL) and dcw determination (5 mL) were harvested as described above (see FIG. 15 for dry cell weight measurements).

### Preparation of Lipophilic Extracts

Care was taken to minimize O₂ and light exposure to acetone:methanol extracts, though samples were not handled anaerobically. Cell pellets from were resuspended in water (950 μL for roux bottle samples and 100 μL for pellets from shake flask cultures), then transferred into a 15 mL glass Sorval centrifuge tubes. Extraction solvent (7:2 acetone:methanol solution; 5 mL or 1.5 mL respectively for roux bottle or shake flask samples) was added and the samples were mixed by pipetting. The tube was centrifuged (10,000×g for 20 min), then the supernatant was transferred to a new 15 mL glass tube. The pelleted cells were extracted a second time, after resuspending cells in water (500 μL or 100 μL respectively for roux bottle or shake flask samples) followed by extraction solvent (4.5 mL or 1.5 mL respectively for roux bottle or shake flask samples). After centrifugation, the supernatants from both extractions were combined. The combined supernatants were partially dried under a stream of N₂ (to a final volume of ˜1-4 mL) to concentrate materials before analysis by HPLC. The concentration of compounds in lipophilic extracts was calculated after correcting for dry cell weight, any dilution prior to extraction and the final volume of sample after drying under N₂.

### HPLC Identification and Quantification of Lipophilic Compounds

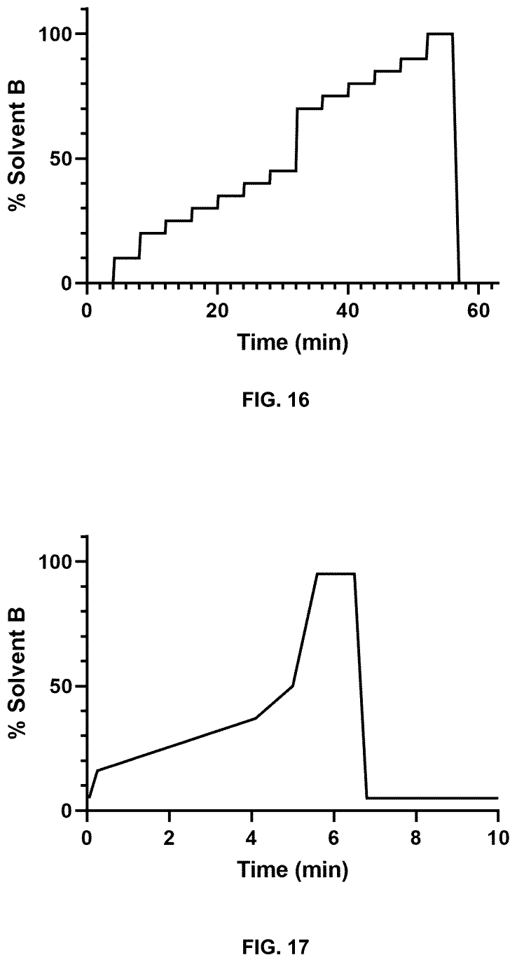

For identification and quantification, the acetone:methanol lipophilic extracts were analyzed via reverse-phase HPLC using a Kinetex 2.6 μm PS C18 100 Å (150×2.1 mm) column (Phenomenex; Torrance, CA) attached to a Shimadzu Nexera XR HPLC system. The mobile phase was a binary gradient (FIG. 16) of Solvent A (70% acetonitrile/30% water) and Solvent B (70% acetonitrile/30% isopropanol) flowing at 0.45 mL/min. Absorbance was measured between 200 and 600 nm using a Shimadzu SPD-M20 Å photodiode array detector. The following commercial standards were used to identify compounds in the lipophilic extracts: β-carotene (Sigma-Aldrich), lycopene (Pharmaceutical Secondary Standard, Certified Reference Material (7.2%); Supelco), zeaxanthin (United States Pharmacopeia Reference Standard), astaxanthin (Sigma-Aldrich), and Coenzyme Q₁₀ (Sigma-Aldrich).

To identify compounds that were not commercially available for use as standards, the eluent from the HPLC was analyzed via mass spectrometry using a Shimadzu triple quadrupole mass spectrometer LCMS-8045. We used positive mode Q3 scans from 450 m/z to 700 m/z around the retention times of unknown HPLC peaks to obtain mass spectra of compounds eluting at such times (FIGS. 4B-D, 9B, and 10B).

### Analysis of Culture Media for PDC and Aromatic Compounds

Extracellular media samples were prepared by centrifuging 1.5 mL of culture at 20,000×g for 2 minutes before passing the supernatant through a 0.2 μm SFCA membrane filter. The filtered media was analyzed using the Shimadzu Nexera XR HPLC system with the photodiode array detector and LCMS-8045 described above. The mobile phase was a binary gradient (FIG. 17) of solvent A (0.200 formic acid in water) and solvent B (methanol) flowing at 0.4 mL/min. The stationary phase was a Phenomenex Kinetex F5 column (2.6 cm pore size, 2.1 mm ID, 150 mm length). Aromatic compounds were identified by multiple-reaction-monitoring (MRM) using the transition ions specified in Table 5, which were obtained from analyzing pure standards as previously described (7). Aromatic compounds were quantified by comparing sample absorbance at specific wavelengths and retention times with known standards, as measured by the photodiode array detector (Table 6).

TABLE 5
Multiple reaction module conditions for HPLC-MS/MS identification
of aromatic compounds in sorghum APL.

| | | | | Transition 1 | | Transition 2 | | Transition 3 | |
|---|---|---|---|---|---|---|---|---|---|
| | | | | m/z, | | m/z, | | m/z, | |
| | MW | DUIS | Parent | collision | | collision | | collision | |
| Compound | (g/mol) | mode | m/z | energy (V) | | energy (V) | | energy (V) | |
| Acetosyringone | 196.2 | + | 197.0 | 155.1, | (−20) | 140.1, | (−28) | 125.1, | (−32) |
| Acetovanillone | 166.2 | + | 167.0 | 125.1, | (−13) | 110.0, | (−23) | N/A | |
| Catechol | 110.1 | − | 109.2 | 91.1, | (24) | 65.1, | (24) | 41.0, | (35) |
| Ferulic acid | 194.2 | − | 193.0 | 134.1, | (17) | 149.1, | (16) | 178.1, | (14) |
| p-Coumaric | 164.0 | − | 163.0 | 119.1, | (16) | 93.1, | (31) | 117.1, | (30) |
| acid | | | | | | | | | |
| PDC | 184.1 | − | 183.1 | 139.0, | (12) | 111, | (15) | 95.1, | (13) |
| p-OH- | 122.1 | − | 121.0 | 92.1, | (25) | 93.1, | (21) | 65.1, | (24) |
| Benzaldehyde | | | | | | | | | |
| p-OH-benzoic | 138.1 | − | 137.0 | 93.0, | (16) | 65.1, | (33) | 75.2, | (30) |
| acid | | | | | | | | | |
| Protocatechuic | 154.1 | − | 153.0 | 109.1, | (17) | 91.1, | (26) | N/A | |
| acid | | | | | | | | | |
| Syringaldehyde | 182.2 | − | 181.1 | 151.1, | (26) | 166.2, | (20) | 123.1, | (27) |
| Syringic acid | 198.2 | − | 197.0 | 182.1, | (14) | 122.8, | (23) | 94.9, | (33) |
| Vanillic acid | 168.1 | − | 167.0 | 152.1, | (19) | 107.9, | (19) | 123.0, | (14) |
| Vanillin | 152.2 | − | 151.0 | 136.0, | (17) | 92.0, | (22) | 108.0, | (24) |

TABLE 6
Photodiode array detection parameters for quantification
of aromatic compounds in sorghum APL.

| | Peak | Wavelength | Retention |
|---|---|---|---|
| | absorbance | used for | time |
| Compound | wavelength (nm) | quantification (nm) | (min) |
| Ferulic acid | 322 | 325 | 3.9 |
| p-Coumaric acid | 312 | 334 | 3.3 |
| PDC | 315 | 325 | 1.2 |
| Protocatechuic acid | 259 | 258 | 1.8 |
| Vanillic acid | 260 | 258 | 2.7 |

Construction of Plasmids for Generating in-Frame Deletions of Saro_1814, Saro_1817, Saro_0236, or Saro_1168

Regions of Novosphingobium aromaticivorans genomic DNA containing ˜1000 bp upstream and downstream of the genes to be deleted were amplified via PCR (see Table 4 for primers). Plasmid pK18msB-MCS1 (a variant of pK18mobsacB (48) in which the multiple cloning site has been removed, and which contains a gene for kanamycin resistance and sacB for sucrose sensitivity) was linearized via PCR as previously described (3). The upstream and downstream flanking regions for each gene were combined with linearized pK18msB-MCS1 using the NEBuilder HiFi Assembly system (New England Biolabs, Ipswich, MA) to produce a plasmid in which the upstream and downstream DNA sequences are adjacent, with no intervening coding region (Table 3). In all cases, a gene's start codon was eliminated; for some genes, a portion of the downstream coding region was retained. The plasmids were transformed into NEB 5-alpha competent Escherichia coli cells (New England Biolabs). The transformed E. coli cells were cultured in LB media+kanamycin, the plasmids were purified using a Qiagen® Plasmid Maxi Kit (Qiagen, Germany), and DNA sequencing was used to confirm the presence of the desired junction between upstream and downstream fragments.

Plasmids for Recombining a Foreign crtW Gene into the N. aromaticivorans Genome

DNA fragments containing the gene coding for the CrtW protein from either Sphingomonas taxi ATCC 55669 (NCBI Accession WP_038660513.1) or Sphingomonas astaxanthinifaciens (NCBI accession WP_211248127.1), with sequences complementary to the upstream and downstream regions of Saro_0236 at the crtW fragments' ends, were ordered as gBlocks from Integrated DNA Technologies (Coralville, IA). Genes encoding CrtW from other organisms were constructed to have codon usage frequencies similar to those of other genes in N. aromaticivorans (calculated from several genes in the genome), but without making the GC content of individual genes too high for the gBlock synthesis process. Sequences of the fragments were (with sequences complementary to the Saro_0236 flanks in lowercase):

Sphingomonas taxi ATCC 55669 crtW:
(SEQ ID NO: 43)
gtcaaccgcgtcaacctgttcaggcaggagccgttgggcATGAGCCCCGATCGGGGGAATACGCGCCACA
GCCTGCTGCTCGCCGCCGCGATCGGTGCGGCCTGGCTCGCCATCCATATTGGCGGCA
TCTTCTTCTGGCAGTGGCGTGCCGCCACGGTGCCGGTCGCACTTCTGCTGATCGTGGT
GCAGGCGTGGCTGAGCACCGGCCTCTTCATCGTCGCGCACGACTGCATGCACGGATC
GTTCGCACCCGGACGCCGGGCGTGGAACGTCGTCGTCGGCACCCTGTGCCTCGGCGC
CTATGCCGGCCTGTCCTATCGCGCGCTCTACCCGATGCACCACGCGCATCATGCCGC
GCCCGGCACCGAACACGATCCCGACTTCCATGCCGCCGCGCCTCGCCGCGCGCTTCC
GTGGTTCGTCCATTTCTTCCGCGGGTACTACACCCATGGCCAGATCCTGCGGATCAC
GCTTGCGGCGATCGTCTACATCCTGCTCGGCGCGTCGCTTCTCAACATCGTGCTGTTC
TGGGCGGTGCCGGCGCTGCTCGCGCTTGCGCAATTGTTTCTGTTCGGCACCTATCTGC
CCCACCGTCACGGCGAGACGCCGTTCGCCGACACGCACAACGCGCGCAGCAACTCG
CTGTCGCCGCTCGCCTCGCTGGCGACCTGCTTCCACTTCGGTGCCTATCACCACGAA
CATCACCTCAGCCCGCAGACTCCGTGGTGGCAGCTCCCGCACATCAAGCGCGGCTG
Acatcacgagcaataccgctgcaactatggcctctacttc
Sphingomonas astaxanthinifaciens crtW:
(SEQ ID NO: 44)
gtcaaccgcgtcaacctgttcaggcaggagccgttgggcATGGCAGAACGCCGTCGCCCGGCCTATATGGC
ACCCATGCTCAGTGATGCGCAGCGCCGTCGCCAGGCGATGATCGGCCTTGGCCTTGC
CGCAGCGATCACCGCAGCCTTCGTCGCGCTTCATGTCTGGTCGGTCTTCTTCCTTCCG
CTTGAAGGAGCAGGCTGGTGGCTTGCGCTTCCGATCGTCGCAGTGCAAACCTGGCTT
AGCGTCGGTCTGTTCATCGTCGCGCATGATGCAATGCATGGCAGCCTTGCACCGGGC
CGCCCTGCGACCAACCTTTTCTGGGGACGGCTTACGCTTCTGCTCTACGCGGGCTTCT
GGTTGGACCGCCTTTCGCCCAAGCATTTCGACCACCACCGCCATGTCGGGACCGAGC
GCGATCCCGATTTCTCGGTCGATCATCCGACCCGCTTCTGGCCCTGGTATTATGCCTT
CATGCGGCGCTATTTCGGGCTTCGCGAATATCTGGTGCTGAACGCGCTGGTGCTGGC
CTACGTGCTGGTGCTGAAGGCGCCGCTCGGCAATCTGCTCCTGTTCTGGGCGCTGCC
CTCGATCCTGTCCTCGATCCAGCTCTTCTATTTCGGCACCTACCTTCCGCACCGGCAC
GAGGACGCGCCCTTCGCCGACCAGCACAATGCCCGCAGCAACGACTTTCCGGTCTG
GCTGTCGCTGCTGACCTGCTTCCACTTCGGCTATCACCGCGAGCATCACCTCAGCCC
CGGCACCCCGTGGTGGCAGCTGCCTCGACGACGGCGAGAGCTTGCACTTCCTGCATG
Acatcacgagcaataccgctgcaactatggcctctacttc

The plasmid that was used to delete Saro_0236 was linearized using primers 5′-CCAACGGCTCCTGCCTGAACAG-3′ (SEQ ID NO:45) and 5′-CATCACGAGCAATACCGCTGCAACTATGG-3′ (SEQ ID NO:46). Each of the crtW DNA fragments was separately combined with this linearized plasmid using the NEBuilder HiFi Assembly system (New England Biolabs) to produce plasmids pK18msB/ΔSaro0236::StaxiW and pK18msB/ΔSaro0236::SastaW, each containing crtW in the Saro_0236 genomic locus (with the start and stop codons for crtW located where those codons for Saro_0236 would normally be located). The plasmids were transformed into NEB 5-alpha competent E. coli (New England Biolabs), and the plasmids were purified and confirmed as described above.

Modifying the N. aromaticivorans Genome

The gene deletion and crtW-containing plasmids were separately mobilized into N. aromaticivorans via conjugation with E. coli S17-1. For conjugation, cultures of E. coli S17-1 harboring the plasmid (in LB containing kanamycin) and N. aromaticivorans (in GluSis) were grown overnight at 30° C. Cultures were diluted and allowed to resume exponential growth before cells were harvested by centrifugation (˜7,000×g for 5 min). Each cell pellet was separately washed in LB, then resuspended together into 90 μL LB containing no added antibiotic. Conjugations were allowed to proceed overnight at 30° C. The following day, the cells were harvested via centrifugation, resuspended into GluSis, and shaken at 200 rpm for >1 h at 30° C. Cells from these cultures were plated onto solid GluSis with kanamycin to select for N. aromaticivorans cells in which the plasmid had integrated into the genome via homologous recombination (single crossovers). Single crossover strains were confirmed through the inability to immediately grow on GluSis containing 10% sucrose.

Single crossover strains were cultured in 5 mL of GluSis containing 10% sucrose and shaken at 30° C. until growth commenced (usually several days), which signified loss of the plasmid from the genome via a second round of homologous recombination. These cultures were streaked onto solid GluSis containing 10% sucrose to generate individual colonies that had lost the plasmid, and plasmid loss was confirmed by the inability to grow on GluSis containing kanamycin. The desired genomic modification was confirmed via PCR and sequencing of isolated genomic DNA.

### Measuring Chemical Oxygen Demand (COD)

COD was measured on filtered and unfiltered samples using COD2 mercury-free high range (20-1,500 mg/L) digestion vials following the manufacturer's protocol (2565115, Hach, Loveland, CO, United States). “Suspended solids” COD was calculated by subtracting the filtered sample COD from the unfiltered sample COD. “Uncharacterized” COD refers to the difference between the total filtered COD and the sum of all calculated CODs for every measured aromatic and organic molecule. The COD for each measured molecule was calculated based on the stoichiometry of how many moles of O₂ would be required to fully oxidize the molecule to CO₂ and H₂O.

### Analysis of Organics in Sorghum APL

Analysis was performed on an Agilent 1260 Infinity II HPLC equipped with an HPX-87H column at 50° C. and a refractive index detector. The mobile phase was 0.02N sulfuric acid flowing at 0.5 mL/min. Samples were centrifuged to remove cells and particulates, decanted, and diluted 1:9 (v/v) with MilliQ water. The sample injection volume was 50 μL. The analytes quantified were glucose, xylose, pyruvic acid, xylitol, cellobiose, succinic acid, lactic acid, propanoic acid, glycerol, formic acid, acetic acid, and ethanol. Concentrations were calculated from a 9-point calibration curve and are reported of the average of two technical replicates.

### REFERENCES

1. U.S. DOE. 2015. Lignocellulose Biomass for Advanced Biofuels and Bioproducts: Workshop Report, DOE/SC-0170. U.S. Department of Energy Office of Science.
2. Perez, J. M., W. S. Kontur, M. Alherech, J. Coplien, S. D. Karlen, S. S. Stahl, T. J. Donohue, and D. R. Noguera. 2019. Funneling aromatic products of chemically depolymerized lignin into 2-pyrone-4-6-dicarboxylic acid with Novosphingobium aromaticivorans. Green Chem. 21:1340-1350.
3. Kontur, W. S., C. A. Bingman, C. N. Olmsted, D. R. Wassarman, A. Ulbrich, D. L. Gall, R. W. Smith, L. M. Yusko, B. G. Fox, D. R. Noguera, J. J. Coon, and T. J. Donohue. 2018. Novosphingobium aromaticivorans uses a Nu-class glutathione S-transferase as a glutathione lyase in breaking the j-aryl ether bond of lignin. J Biol. Chem. 293:4955-4968.
4. Cecil, J. H., D. C. Garcia, R. J. Giannone, and J. K. Michener. 2018. Rapid, parallel identification of catabolism pathways of lignin-derived aromatic compounds in Novosphingobium aromaticivorans. Appl Environ Microbiol. 84:e01185-18.
5. Presley, G. N., A. Z. Werner, R. Katahira, D. C. Garcia, S. J. Haugen, K. J. Ramirez, R. J. Giannone, G. T. Beckham, and J. K. Michener. 2021. Pathway discovery and engineering for cleavage of a 3-1 lignin-derived biaryl compound. Metabolic Engineering. 65:1-10.
6. Michinobu, T., M. Bito, Y. Yamada, M. Tanimura, Y. Katayama, E. Masai, M. Nakamura, Y. Otsuka, S. Ohara, and K. Shigehara. 2009. Fusible, elastic, and biodegradable polyesters of 2-pyrone-4,6-picarboxylic acid (PDC). Polym J. 41:1111-1116.
7. Perez, J. M., W. S. Kontur, C. Gehl, D. M. Gille, Y. Ma, A. V. Niles, G. Umana, T. J. Donohue, and D. R. Noguera. 2021. Redundancy in Aromatic O-Demethylation and Ring-Opening Reactions in Novosphingobium aromaticivorans and Their Impact in the Metabolism of Plant-Derived Phenolics. Appl Environ Microbiol. 87:e02794-20.
8. Berman, J., U. Zorrilla-López, G. Farré, C. Zhu, G. Sandmann, R. M. Twyman, T. Capell, and P. Christou. 2015. Nutritionally important carotenoids as consumer products. Phytochem Rev. 14:727-743.
9. Mussagy, C. U., J. Winterburn, V. C. Santos-Ebinuma, and J. F. B. Pereira. 2019. Production and extraction of carotenoids produced by microorganisms. Appl Microbiol Biotechnol. 103:1095-1114.
10. Bogacz-Radomska, L., J. Harasym, and A. Piwowar. 2020. 10—Commercialization aspects of carotenoids. In: Galanakis C M, editor. Carotenoids: Properties, Processing and Applications. Academic Press. pp. 327-357.
11. Stachowiak, B., and P. Szulc. 2021. Astaxanthin for the Food Industry. Molecules. 26:2666.
12. Álvarez, R., B. Vaz, H. Gronemeyer, and Á. R. de Lera. 2014. Functions, therapeutic applications, and synthesis of retinoids and carotenoids. Chem. Rev. 114:1-125.
13. Siddaramappa, S., V. Viswanathan, S. Thiyagarajan, and A. Narjala. 2018. Genomewide characterisation of the genetic diversity of carotenogenesis in bacteria of the order Sphingomonadales. Microb Genom. 4.
14. Linz, A. M., Y. Ma, S. Scholz, D. R. Noguera, and T. J. Donohue. 2022. iNovo479: Metabolic modeling provides a roadmap to optimize bioproduct yield from deconstructed lignin aromatics by Novosphingobium aromaticivorans. Metabolites. 12:366.
15. Balkwill, D. L., G. R. Drake, R. H. Reeves, J. K. Fredrickson, D. C. White, D. B. Ringelberg, D. P. Chandler, M. F. Romine, D. W. Kennedy, and C. M. Spadoni. 1997. Taxonomic study of aromatic-degrading bacteria from deep-terrestrial-subsurface sediments and description of Sphingomonas aromaticivorans sp. nov., Sphingomonas subterranea sp. nov., and Sphingomonas stygia sp. nov. Int J Syst Bacteriol. 47:191-201.
16. Lee, S. Q. E., T. S. Tan, M. Kawamukai, and E. S. Chen. 2017. Cellular factories for coenzyme Q10 production. Microb Cell Fact. 16:39.
17. Shukla, S., and K. K. Dubey. 2018. CoQ10 a super-vitamin: review on application and biosynthesis. 3 Biotech. 8:249.
18. Jeya, M., H.-J. Moon, J.-L. Lee, I.-W. Kim, and J.-K. Lee. 2010. Current state of coenzyme Q10 production and its applications. Appl Microbiol Biotechnol. 85:1653-1663.
19. Cirilli, I., E. Damiani, P. V. Dludla, I. Hargreaves, F. Marcheggiani, L. E. Millichap, P. Orlando, S. Silvestri, and L. Tiano. 2021. Role of Coenzyme Q10 in Health and Disease: An Update on the Last 10 Years (2010-2020). Antioxidants. 10:1325.
20. Cluis, C. P., A. M. Burja, and V. J. J. Martin. 2007. Current prospects for the production of coenzyme Q10 in microbes. Trends Biotechnol. 25:514-521.
21. Jenkins, C. L., A. G. Andrewes, T. J. McQuade, and M. P. Starr. 1979. The pigment of Pseudomonas paucimobilis is a carotenoid (nostoxanthin), rather than a brominated aryl-polyene (xanthomonadin). Current Microbiology. 3:1-4.
22. Setiyono, E., Heriyanto, D. Pringgenies, Y. Shioi, Y. Kanesaki, K. Awai, and T. H. P. Brotosudarmo. 2019. Sulfur-containing carotenoids from a marine coral symbiont Erythrobacter flavus strain KJ5. Marine Drugs. 17:349.
23. Zhu, L., X. Wu, O. Li, C. Qian, and H. Gao. 2012. Cloning and characterization of genes involved in nostoxanthin biosynthesis of Sphingomonas elodea ATCC 31461. PLoS One. 7:e35099.
24. Asker, D., T. Beppu, and K. Ueda. 2007. Sphingomonas astaxanthinfaciens sp. nov., a novel astaxanthin-producing bacterium of the family Sphingomonadaceae isolated from Misasa, Tottori, Japan. FEMS Microbiology Letters. 273:140-148.
25. Ma, T., Y. Zhou, X. Li, F. Zhu, Y. Cheng, Y. Liu, Z. Deng, and T. Liu. 2016. Genome mining of astaxanthin biosynthetic genes from Sphingomonas sp. ATCC 55669 for heterologous overproduction in Escherichia coli. Biotechnol J. 11:228-237.
26. Wan, X., X.-R. Zhou, G. Moncalian, L. Su, W.-C. Chen, H.-Z. Zhu, D. Chen, Y.-M. Gong, F.-H. Huang, and Q.-C. Deng. 2021. Reprogramming microorganisms for the biosynthesis of astaxanthin via metabolic engineering. Progress in Lipid Research. 81:101083.
27. Sanchez, S., B. Ruiz, R. Rodriguez-Sanoja, and L. B. Flores-Cotera. 2013. 9—Microbial production of carotenoids. In: McNeil B, D Archer, I Giavasis, L Harvey, editors. Microbial Production of Food Ingredients, Enzymes and Nutraceuticals. Woodhead Publishing. pp. 194-233.
28. Kim, J. S., Y. Y. Lee, and T. H. Kim. 2016. A review on alkaline pretreatment technology for bioconversion of lignocellulosic biomass. Bioresource Technology. 199:42-48.
29. Yoshida, H., Y. Kotani, K. Ochiai, and K. Araki. 1998. Production of ubiquinone-10 using bacteria. The Journal of General and Applied Microbiology. 44:19-26.
30. Yamane, Y., K. Higashida, Y. Nakashimada, T. Kakizono, and N. Nishio. 1997. Influence of Oxygen and Glucose on Primary Metabolism and Astaxanthin Production by Phaffia rhodozyma in Batch and Fed-Batch Cultures: Kinetic and Stoichiometric Analysis. Appl Environ Microbiol. 63:4471-4478.
31. Fredrickson, J. K., F. J. Brockman, D. J. Workman, S. W. Li, and T. O. Stevens. 1991. Isolation and characterization of a subsurface bacterium capable of growth on toluene, naphthalene, and other aromatic compounds. Appl. Environ. Microbiol. 57:796-803.
32. Frengova, G. I., and D. M. Beshkova. 2009. Carotenoids from Rhodotorula and Phaffia: yeasts of biotechnological importance. Journal of Industrial Microbiology and Biotechnology. 36:163.
33. Fan, J., W. Xu, X. Xu, and Y. Wang. 2022. Production of Coenzyme Q10 by microbes: an update. World J Microbiol Biotechnol. 38:194.
34. Harwood, C. S., and J. Gibson. 1988. Anaerobic and aerobic metabolism of diverse aromatic compounds by the photosynthetic bacterium Rhodopseudomonas palustris. Appl Environ Microbiol. 54:712-717.
35. Hainal, A. R., A. M. Capraru, I. Volf, and V. Popa. 2012. Lignin as a carbon source for the cultivation of some Rhodotorula species. Cellulose Chemistry and Technology. 46:87-96.
36. Chen, Z., and C. Wan. 2021. Microbial conversion of lignin-based compounds into carotenoids by rhodococci. Appl Biochem Biotechnol. 193:3442-3453.
37. Li, X., M. Li, Y. Pu, A. J. Ragauskas, N. Tharayil, J. Huang, and Y. Zheng. 2021. Degradation of aromatic compounds and lignin by marine protist Thraustochytrium striatum. Process Biochemistry. 107:13-17.
38. Xu, B., Q. Li, Y. Pu, S. Xie, A. J. Ragauskas, J. Arreola-Vargas, Z.-H. Liu, and J. S. Yuan. 2022. A unique bacterial pelletized cultivation platform in Rhodococcus opacus PD630 enhanced lipid productivity and simplified harvest for lignin bioconversion. ACS Sustainable Chem. Eng. 10:1083-1092.
39. Ravi, K., O. Y. Abdelaziz, M. Nobel, J. Garcia-Hidalgo, M. F. Gorwa-Grauslund, C. P. Hulteberg, and G. Liden. 2019. Bacterial conversion of depolymerized Kraft lignin. Biotechnology for Biofuels. 12:56.
40. Guo, P., K. Mochidzuki, W. Cheng, M. Zhou, H. Gao, D. Zheng, X. Wang, and Z. Cui. 2011. Effects of different pretreatment strategies on corn stalk acidogenic fermentation using a microbial consortium. Bioresour Technol. 102:7526-7531.
41. Kondaveeti, S., A. Bisht, R. Pagolu, C. Lai, R. Lestari, A. Kumar, D. Das, V. C. Kalia, and J.-K. Lee. 2022. Mild alkaline pretreatment of rice straw as a feedstock in microbial fuel cells for generation of bioelectricity. Indian J Microbiol. 62:447-455.
42. Linger, J. G., D. R. Vardon, M. T. Guarnieri, E. M. Karp, G. B. Hunsinger, M. A. Franden, C. W. Johnson, G. Chupka, T. J. Strathmann, P. T. Pienkos, and G. T. Beckham. 2014. Lignin valorization through integrated biological funneling and chemical catalysis. Proceedings of the National Academy of Sciences. 111:12013-12018.
43. Zhang, K., R. Xu, A. E.-F. Abomohra, S. Xie, Z. Yu, Q. Guo, P. Liu, L. Peng, and X. Li. 2019. A sustainable approach for efficient conversion of lignin into biodiesel accompanied by biological pretreatment of corn straw. Energy Conversion and Management. 199:111928.
44. Salvachna, D., E. M. Karp, C. T. Nimlos, D. R. Vardon, and G. T. Beckham. 2015. Towards lignin consolidated bioprocessing: simultaneous lignin depolymerization and product generation by bacteria. Green Chem. 17:4951-4967.
45. Umana, G. E., J. M. Perez, F. Unda, C.-Y. Lin, C. Sener, S. D. Karlen, S. D. Mansfield, A. Eudes, J. Ralph, T. J. Donohue, and D. R. Noguera. 2022. Biological funneling of phenolics from transgenic plants engineered to express the bacterial 3-dehydroshikimate dehydratase (qsuB) gene. Frontiers in Chemical Engineering. 4.
46. Shikinaka, K., Y. Otsuka, M. Nakamura, E. Masai, and Y. Katayama. 2018. Utilization of lignocellulosic biomass via novel sustainable process. J Oleo Sci. 67:1059-1070.
47. Fredrickson, J. K., D. L. Balkwill, G. R. Drake, M. F. Romine, D. B. Ringelberg, and D. C. White. 1995. Aromatic-degrading Sphingomonas isolates from the deep subsurface. Appl. Environ. Microbiol. 61:1917-1922.
48. Schafer, A., A. Tauch, W. Jäger, J. Kalinowski, G. Thierbach, and A. Pühler. 1994. Small mobilizable multi-purpose cloning vectors derived from the Escherichia coli plasmids pK18 and pK19: selection of defined deletions in the chromosome of Corynebacterium glutamicum. Gene. 145:69-73.
49. Sistrom, W. R. 1962. The kinetics of the synthesis of photopigments in Rhodopseudomonas spheroides. J. Gen. Microbiol. 28:607-616.
50. Simon, R., U. Priefer, and A. Pühler. 1983. A Broad Host Range Mobilization System for In Vivo Genetic Engineering: Transposon Mutagenesis in Gram Negative Bacteria. Nat Biotech. 1:784-791.
51. Price M N, Arkin A P. PaperBLAST: Text Mining Papers for Information about Homologs. mSystems. 2017 Aug. 15; 2(4):e00039-17.

1. A recombinant microorganism comprising one or more modifications with respect to a corresponding microorganism not comprising the one or more modifications, wherein the one or more modifications comprise any one or more of:
a modification that decreases phytoene synthase (CrtB) activity with respect to the corresponding microorganism;
a modification that decreases lycopene-forming phytoene desaturase (CrtI) activity with respect to the corresponding microorganism;
a modification that decreases lycopene cyclase (CrtY) activity with respect to the corresponding microorganism;
a modification that decreases beta-carotene hydroxylase (CrtZ) activity with respect to the corresponding microorganism;
a modification that decreases 2,2′-beta hydroxylase (CrtG) activity with respect to the corresponding microorganism; and
a modification that increases beta-carotene ketolase (CrtW) activity with respect to the corresponding microorganism.

2. The recombinant microorganism of claim 1, wherein:
the modification that decreases phytoene synthase phytoene synthase (CrtB) activity comprises a modification to a gene encoding a phytoene synthase phytoene synthase (CrtB) comprising a sequence having at least 80%, at least 85%, at least 90%, at least 95%, or at least 99% sequence identity to SEQ ID NO:2;
the modification that decreases lycopene-forming phytoene (CrtI) desaturase activity comprises a modification to a gene encoding a lycopene-forming phytoene desaturase (CrtI) comprising a sequence having at least 80%, at least 85%, at least 90%, at least 95%, or at least 99% sequence identity to SEQ ID NO:4;
the modification that decreases lycopene cyclase (CrtY) activity comprises a modification to a gene encoding a lycopene cyclase (CrtY) comprising a sequence having at least 80%, at least 85%, at least 90%, at least 95%, or at least 99% sequence identity to SEQ ID NO:6;
the modification that decreases beta-carotene hydroxylase (CrtZ) activity comprises a modification to a gene encoding a beta-carotene hydroxylase (CrtZ) comprising a sequence having at least 80%, at least 85%, at least 90%, at least 95%, or at least 99% sequence identity to SEQ ID NO:8; and/or
the modification that decreases 2,2′-beta hydroxylase (CrtG) activity comprises a modification to a gene encoding a 2,2′-beta hydroxylase (CrtG) comprising a sequence having at least 80%, at least 85%, at least 90%, at least 95%, or at least 99% sequence identity to SEQ ID NO:10.

3. The recombinant microorganism of claim 1, comprising any one or more of:
the modification that decreases phytoene synthase phytoene synthase (CrtB) activity;
the modification that decreases lycopene-forming phytoene desaturase (CrtI) activity;
the modification that decreases lycopene cyclase (CrtY) activity;
the modification that decreases beta-carotene hydroxylase (CrtZ) activity; and
the modification that decreases 2,2′-beta hydroxylase (CrtG) activity.

4. (canceled)

5. The recombinant microorganism of claim 1, comprising the modification that decreases phytoene synthase phytoene synthase (CrtB) activity with respect to the corresponding microorganism.

6. The recombinant microorganism of claim 5, wherein the recombinant microorganism exhibits increased accumulation of coenzyme Q₁₀ (CoQ₁₀) with respect to the corresponding microorganism.

7. The recombinant microorganism of claim 1, comprising the modification that decreases lycopene cyclase (CrtY) activity.

8. The recombinant microorganism of claim 7, wherein the recombinant microorganism exhibits increased accumulation of lycopene with respect to the corresponding microorganism.

9. The recombinant microorganism of claim 1, comprising the modification that decreases 2,2′-beta hydroxylase (CrtG) activity.

10. The recombinant microorganism of claim 9, wherein the recombinant microorganism exhibits increased accumulation of zeaxanthin with respect to the corresponding microorganism.

11. The recombinant microorganism of claim 9, comprising the modification that decreases beta-carotene hydroxylase (CrtZ) activity.

12. The recombinant microorganism of claim 11, wherein the recombinant microorganism exhibits increased accumulation of beta-carotene with respect to the corresponding microorganism.

13. The recombinant microorganism of claim 9, comprising the modification that increases beta-carotene ketolase (CrtW) activity.

14. The recombinant microorganism of claim 13, wherein the modification that increases beta-carotene ketolase (CrtW) activity comprises a recombinant gene encoding a beta-carotene ketolase (CrtW) comprising a sequence having at least 80%, at least 85%, at least 90%, at least 95%, or at least 99% sequence identity to SEQ ID NO: 12 or SEQ ID NO:14.

15. The recombinant microorganism of claim 13, wherein the recombinant microorganism exhibits increased accumulation of at least one of adonixanthin and astaxanthan with respect to the corresponding microorganism.

16. The recombinant microorganism of claim 1, further comprising:
a modification that decreases 2-pyrone-4,6-dicarboxylic acid (PDC) hydrolase (LigI) activity with respect to the corresponding microorganism;
a modification that decreases 4-carboxy-2-hydroxy-6-methoxy-6-oxohexa-2,4-dienoate (CHMOD) methyl esterase (DesC) activity with respect to the corresponding microorganism;
a modification that decreases 4-carboxy-2-hydroxy-6-methoxy-6-oxohexa-2,4-dienoate (CHMOD) cis-trans isomerase (DesD) activity with respect to the corresponding microorganism; and/or
a modification that decreases vanillate/3-O-methylgallate O-demethylase (DmtS) activity with respect to the corresponding microorganism.

17. (canceled)

18. The recombinant microorganism of claim 16, wherein the recombinant microorganism exhibits increased accumulation of 2-pyrone-4,6-dicarboxylic acid (PDC) with respect to the corresponding microorganism.

19. The recombinant microorganism of claim 1, wherein the recombinant microorganism is from the genus Novosphingobium.

20. The recombinant microorganism of claim 1, wherein the recombinant microorganism is Novosphingobium aromaticivorans.

21. A method for producing a compound comprising culturing the recombinant microorganism of claim 1 in a medium comprising a plant-derived phenolic.

22-25. (canceled)

26. The recombinant microorganism of claim 3, wherein:
the modification that decreases phytoene synthase phytoene synthase (CrtB) activity comprises a modification to a gene encoding a phytoene synthase phytoene synthase (CrtB) comprising a sequence having at least 95% sequence identity to SEQ ID NO:2;
the modification that decreases lycopene-forming phytoene (CrtI) desaturase activity comprises a modification to a gene encoding a lycopene-forming phytoene desaturase (CrtI) comprising a sequence having at least 95% sequence identity to SEQ ID NO:4;
the modification that decreases lycopene cyclase (CrtY) activity comprises a modification to a gene encoding a lycopene cyclase (CrtY) comprising a sequence having at least 95% sequence identity to SEQ ID NO:6;
the modification that decreases beta-carotene hydroxylase (CrtZ) activity comprises a modification to a gene encoding a beta-carotene hydroxylase (CrtZ) comprising a sequence having at least 95% sequence identity to SEQ ID NO:8; and/or
the modification that decreases 2,2′-beta hydroxylase (CrtG) activity comprises a modification to a gene encoding a 2,2′-beta hydroxylase (CrtG) comprising a sequence having at least 95% sequence identity to SEQ ID NO:10.
